(19) United States
(12) Patent Application Publication
(10) Pub. No.: US 2017/0305943 A1
(43) Pub. Date: Oct. 26, 2017

(54) COMPOUNDS FOR THE TREATMENT OF BACTERIAL INFECTIONS

(71) Applicant: TAXIS Pharmaceuticals, Inc., North Brunswick, NJ (US)

(72) Inventors: David Ryall Brown, Yarnton, Oxfordshire (GB); Ian Collins, Yarnton, Oxfordshire (GB); Lloyd George Czaplewski, Yarnton, Oxfordshire (GB); David John Haydon, Yarnton, Oxfordshire (GB); Penelope Anne Mayes, Yarnton, Oxfordshire (GB); Jeffrey Peter Mitchell, Yarnton, Oxfordshire (GB); James T Palmer, Oxfordshire (GB); Neil Robert Stokes, Oxfordshire (GB)

(73) Assignee: TAXIS Pharmaceuticals, Inc., North Brunswick, NJ (US)

(21) Appl. No.: 15/313,046

(22) PCT Filed: May 21, 2015
(86) PCT No.: PCT/US2015/032052

§ 371 Date: Nov. 21, 2016

(30) Foreign Application Priority Data
May 21, 2014 (AU) 2014901885
May 22, 2014 (AU) 2014901912

(51) Int. Cl. C07F 9/653; C07C 235/46; C07D 413/12; C07D 307/42; C07D 285/08; C07D 277/64; C07D 277/32; C07D 277/24; C07D 271/06; C07D 263/34; C07D 263/32; C07D 249/06; C07D 239/26; C07D 231/12; C07D 213/64; C07C 255/54; C07C 251/48; C07C 251/04; C07D 417/04; C07D 513/04

(52) U.S. Cl. CPC C07F 9/653; C07D 513/04; C07C 235/46; C07D 413/12; C07D 307/42; C07D 285/08; C07D 277/64; C07D 277/32; C07D 277/24; C07D 271/06; C07D 263/34; C07D 263/32; C07D 249/06; C07D 239/26; C07D 231/12; C07D 213/64; C07C 255/54; C07C 251/48; C07C 251/04; C07D 417/04

(57) ABSTRACT

Compounds and methods are provided for treating bacterial infections.

### RELATED APPLICATIONS

This application claims priority to Australian Provisional Patent Application No. 2014901885, filed May 21, 2014, and Australian Provisional Patent Application No. 2014901912, filed May 22, 2014, the entirety of which is incorporated herein by reference.

### TECHNICAL FIELD

The present invention relates to compounds for the treatment of bacterial infections. More particularly, the invention relates to compounds that demonstrate antibacterial activity, their use in methods for the treatment of bacterial infections, a new class of compounds per se, pharmaceutical compositions comprising them and processes for their manufacture.

### BACKGROUND

Bacterial infections are responsible for many human conditions and illnesses and in severe cases can be life-threatening. Many classes of antibacterials have been developed since the discovery of penicillin including the cephalosporins, fluoroquinolines and quinolines, monobactams, rifamycins, aminoglycosides, glycopeptides, macrolides and so on. However, the emergence of bacterial resistance to known classes of antibacterials is of increasing concern as is the low output of novel antibacterial drug classes over the past few decades. Thus there is international recognition of the long-felt and ongoing need for new antibacterials, particularly to address the issue of emerging resistance (Silver, L. L., Challenges of Antibacterial Discovery, Clinical Microbiology Reviews, January 2011, Vol. 24, No. 1, p71-109).

Clostridium difficile (CD) is a Gram-positive anaerobic bacterial pathogen. Clostridium difficile infections (CDIs) are considered to be one of the most important causes of health care-associated infections (HAIs). In early 2001, there was an observed increase in the severity of CDI and the number of patients diagnosed in the US with intestinal infections resulting from CD. In 2002 severe and recurrent outbreaks of CD occurred in Canada. The cause of these outbreaks has since been associated with the now highly prevalent and virulent strain NAP1/BI/027 and this epidemic strain has spread to England and parts of continental Europe.

Studies have demonstrated that 50% or more of hospital patients infected with CD are symptomless carriers. However, for those patients who have symptoms, these symptoms include mild to moderate diarrhoea, nosocominal antibiotic associated diarrhoea, community acquired diarrhoea, fulminant and even fatal pseudomembranous colitis. Complications of severe CD colitis include dehydration, electrolyte disturbances, hypoalbuminemia, toxic megacolon, bowel perforation, hypotension, renal failure, systemic inflammatory response syndrome, sepsis and death (Cohen, S. H., et. al. Clinical Practice Guidelines for Clostridium difficile Infection in Adults: 2010 Update by the Society for Healthcare Epidemiology of America (SHEA) and the Infectious Diseases Society of America (IDSA), Infection Control and Hospital Epidemiology May 2010 Vol. 31. No. 5, p 431-455).

CDI occur in the gastrointestinal tract. The particular challenges associated with treating CDI include the disruption or suppression of normal bowel flora by the administration of antibacterials which enables the CDI to flourish. Accordingly, CDIs infections are often associated with antibacterial use and increased susceptibility is commonly observed with longer exposure to antibacterial therapy and exposure to multiple antibacterials. Other risk factors for CDIs include advanced age, duration of hospitalisation, cancer therapy, immunocompromised patients such as those with human immunodeficiency virus (HIV) and patients undergoing abdominal or gastrointestinal surgery or manipulation of the gastrointestinal tract such as tube feeding. Also of concern is the recent observance of CDIs among previously low risk populations such as healthy peripartum women.

Although current antibacterial therapy for CDI infections comprises administration of metronidazole and/or vancomycin, these compounds disrupt intestinal flora leaving patients susceptible to recurrences of bacterial infections. Furthermore, the reduced effectiveness of metronidazole has been observed and it is not recommended beyond the first recurrence or for long-term chronic therapy due to the potential for cumulative neurotoxicity.

The present inventors have discovered a class of compounds with demonstrated activity against CD. The class of compounds includes compounds previously described in WO2007/107758, WO2009/037485, WO2009/040507 and WO2012/142671 (each of which is incorporated by reference) as well as novel compounds per se.

### SUMMARY

Accordingly one embodiment provides a method for treating a Clostridium difficile infection comprising administration of a compound of Formula (I″):

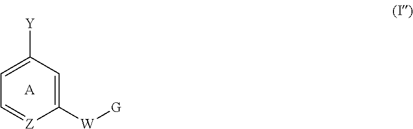

to a patient with said infection or a pharmaceutically acceptable salt, racemate, isomer, diastereoisomer, enantiomer, hydrate, solvate, N-oxide, pharmaceutically acceptable derivative or prodrug thereof, wherein:

A ring is optionally substituted with one or more substituents;

G is

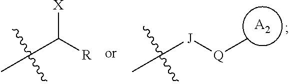

Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;

Z is CH or N;

W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and

R is optionally substituted and is selected from C₁₋₁₂alkyl, C₂₋₁₂alkenyl, C₂₋₁₂alkynyl, C₃₋₈cycloalkyl, C₆₋₁₀aryl and 4-10-membered heterocycles;

Q is selected from O, CH₂ or NR₇ where R₇ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

J is an optionally substituted linker selected from C₁₋₁₂alkyl, C₂₋₁₂alkenyl, C₂₋₁₂alkynyl, optionally interrupted by an ether linkage; and

A₂ is optionally substituted and is selected from C₆₋₁₀aryl and 5-10-membered heterocycles.

One embodiment provides a method for treating a Clostridium difficile infection comprising administration of a compound of Formula (I) or Formula (II):

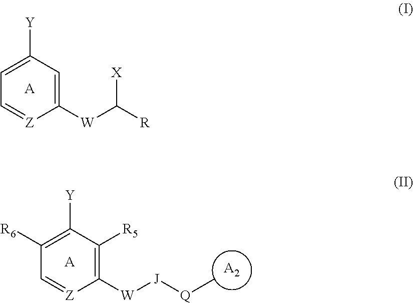

to a patient with said infection or a pharmaceutically acceptable salt, racemate, isomer, diastereoisomer, enantiomer, hydrate, solvate, N-oxide, pharmaceutically acceptable derivative or prodrug thereof, wherein:

A ring is optionally substituted with one or more substituents;

Y is selected from CONR₁R₂ and C(═NR₃)NR₁R₂ where R₁ and R₂ are independently selected from H or optionally substituted C₁ alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;

Z is CH or N;

W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and

R is optionally substituted and is selected from C₁₋₁₂alkyl, C₂₋₁₂alkenyl, C₂₋₁₂alkynyl, C₃₋₈cycloalkyl, C₆₋₁₀aryl and 4-10-membered heterocycles;

R₅ is selected from F or Cl;

R₆ is H or an optional substituent;

Q is selected from O, CH₂ or NR₇ where R₇ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

J is an optionally substituted linker selected from C₁₋₁₂alkyl, C₂₋₁₂alkenyl, C₂₋₁₂alkynyl, optionally interrupted by an ether linkage; and

A₂ is optionally substituted and is selected from C₆₋₁₀aryl and 5-10-membered heterocycles.

One embodiment provides a method for treating a Clostridium difficile infection comprising administration of a compound of Formula (I) to a patient with said infection

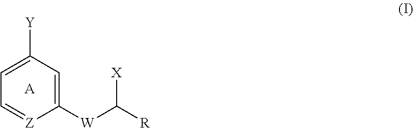

or a pharmaceutically acceptable salt, racemate, isomer, diastereoisomer, enantiomer, hydrate, solvate, N-oxide, pharmaceutically acceptable derivative or prodrug thereof, wherein

A ring is optionally substituted with one or more substituents;

Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, SO₂C₁₋₆alkyl or R₃ joins together with R₁ or R₂ to form a —C(═O)—O— cyclic linking unit;

Z is C or N;

W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and

R is optionally substituted and is selected from C₁₋₁₂alkyl, C₂₋₁₂alkenyl, C₂₋₁₂alkynyl, C₃₋₈cycloalkyl, C₆₋₁₀aryl and 4-10-membered heterocycles.

One embodiment provides a compound of formula (Ia)

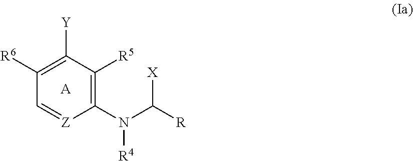

or a salt, racemate, isomer, diastereoisomer, enantiomer, hydrate, solvate, N-oxide, pharmaceutically acceptable derivative or prodrug thereof, wherein

A ring is optionally substituted with one or more substituents;

Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;

Z is C or N;

R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

R₅ and R₆ are independently selected from F or Cl;

X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and

R is optionally substituted and is selected from C₁₋₁₂alkyl, C₂₋₁₂alkenyl, C₂₋₁₂alkynyl, C₃₋₈cycloalkyl, C₆₋₁₀aryl and 4-10-membered heterocycles.

One embodiment provides a compound of formula (II)

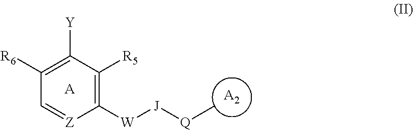

or a salt, racemate, isomer, diastereoisomer, enantiomer, hydrate, solvate, N-oxide, pharmaceutically acceptable derivative or prodrug thereof, wherein

A ring is optionally substituted with one or more substituents;

Y is selected from CONR₁R₂ and C(═NR₃)NR₁R₂ where R₁ and R₂ are independently selected from H or optionally substituted C₁ alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;

R₅ is selected from F or Cl;

R₆ is H or an optional substituent;

Z is C or N;

W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

Q is selected from O, CH₂ or NR₇ where R₇ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

J is an optionally substituted linker selected from C₁₋₁₂alkyl, C₂₋₁₂alkenyl, C₂₋₁₂alkynyl, optionally interrupted by an ether linkage; and

A₂ is optionally substituted and is selected from C₆₋₁₀aryl and 5-10-membered heterocycles.

One embodiment provides a compound of formula (II)

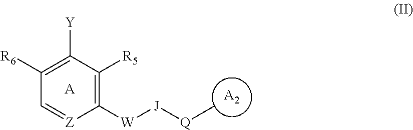

or a salt, racemate, isomer, diastereoisomer, enantiomer, hydrate, solvate, N-oxide, pharmaceutically acceptable derivative or prodrug thereof, wherein

A ring is optionally substituted with one or more substituents;

Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;

R₅ is selected from F or Cl;

R₆ is H or an optional substituent;

Z is C or N;

W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

Q is selected from O, S, CH₂ or NR₇ where R₇ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

J is an optionally substituted linker selected from C₁₋₁₂alkyl, C₂₋₁₂alkenyl, C₂₋₁₂alkynyl, optionally interrupted by an ether linkage; and

A₂ is optionally substituted and is selected from C₆₋₁₀aryl and 5-10-membered heterocycles.

One embodiment provides a composition comprising a compound of Formula (I″), Formula (I) or Formula (II) or a salt, racemate, isomer, diastereoisomer, enantiomer, hydrate, solvate, N-oxide, pharmaceutically acceptable derivative or prodrug thereof. In a particular embodiment the composition is a pharmaceutical composition and the salts are pharmaceutically acceptable. Preferably the pharmaceutical composition is for use in the treatment of a Clostridium difficile infection.

One embodiment provides a method for the treatment of a bacterial infection comprising administration of a compound of Formula (I″), Formula (I) or Formula (II) or a pharmaceutically acceptable salt, racemate, isomer, diastereoisomer, enantiomer, hydrate, solvate, N-oxide, pharmaceutically acceptable derivative or prodrug thereof to a patient with said infection. In a particular embodiment, the bacterial infection is a Clostridium difficile infection.

One embodiment provides a compound of Formula (I″), Formula (I) or Formula (II) or a salt, racemate, isomer, diastereoisomer, enantiomer, hydrate, solvate, N-oxide, pharmaceutically acceptable derivative or prodrug thereof for treating a bacterial infection. In a particular embodiment the, bacterial infection is a Clostridium difficile infection.

One embodiment provides the use of a compound of Formula (I″), Formula (I) or Formula (II) or a salt, racemate, isomer, diastereoisomer, enantiomer, hydrate, solvate, N-oxide, pharmaceutically acceptable derivative or prodrug thereof in the preparation of a medicament for the treatment of a bacterial infection in a subject (e.g., a patient). In a particular embodiment the, bacterial infection is a Clostridium difficile infection.

### DETAILED DESCRIPTION

The present inventors have discovered a class of compounds with demonstrated activity against Clostridium difficile. The inventors have also discovered compounds with demonstrated activity against the hyper-virulent Clostridium difficile strain NAP1/BI/027 which has been associated with severe outbreaks of infection.

### Methods of Treatment

In one embodiment there is provided a method for treating a Clostridium difficile infection with a compound of Formula (I″), Formula (I) or a pharmaceutically acceptable salt, racemate, isomer, diastereoisomer, enantiomer, hydrate, solvate, N-oxide, pharmaceutically acceptable derivative or prodrug thereof wherein:

A ring is an optionally substituted phenyl;

Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;

X is an optionally substituted C₁₋₆alkyl, C₂₋₆alkenyl or C₂₋₆alkynyl; and

R is optionally substituted and is selected from C₃₋₈cycloalkyl, C₆₋₁₀aryl, 4-10-membered heterocycles, or C₆₋₁₂alkyl where C₆₋₁₂alkyl may be straight chain or branched, saturated or unsaturated.

In another embodiment A ring is optionally substituted with one, two or three substituents independently selected from halo, hydroxyl, C₁₋₃alkyl, C₂₋₃alkenyl, C₂₋₃alkynyl, C₁₋₃alkoxyl, C₁₋₃alkylhalo, C₁₋₃alkoxylhalo, CN, NH₂, NH(C₁₋₃alkyl), N(C₁₋₃alkyl)₂ and NO₂.

In one embodiment Y is CONR₁R₂.

In another embodiment R₁ and R₂ are each H.

In one embodiment R is an optionally substituted C₆₋₁₀aryl or an optionally substituted 4-10-membered monocyclic or bicyclic heterocycle. Preferably R is optionally substituted and is selected from monocyclic C₆aryl such as phenyl, bicyclic C₁₀aryl such as naphthyl, a 5-membered monocyclic heterocycle, a 6-membered monocyclic heterocycle, a 9-membered bicyclic heterocycle and a 10-membered bicyclic heterocycle. Even more preferably R is an optionally substituted phenyl or an optionally substituted 5-6 membered monocyclic heteroaryl. Optionally substituted 5-membered monocyclic heteroaryls are particularly preferred.

Preferred optional substituents for R include but are not limited to one or more substituents independently selected from halo, hydroxyl, NO₂, CN, C₁₋₆alkyl such as methyl, ethyl and propyl, C₂₋₆alkenyl such as ethenyl and propenyl, C₂₋₆alkynyl such as ethynyl and propynyl, C₁₋₆alkoxy such as methoxy, ethoxy and propoxy, haloC₁₋₆alkyl such as CHF₂ and CF₃, haloC₁₋₆alkoxy such as OCHF₂ and OCF₃, amino such as NH₂, NH(C₁₋₆alkyl) and N(C₁₋₆alkyl)₂, R₄, C₁₋₆alkyl-R₄, C₂₋₆alkenyl-R₄, C₂₋₆alkynyl-R₄ where R₄ is selected from C₃₋₈cycloalkyl, C₆₋₁₀aryl such as phenyl, 4-10-membered heterocycles such as 5-membered, 6-membered, 9-membered or 10-membered heterocycles, wherein each optional substituent having an available substitutable position may be further optionally substituted.

In one embodiment, R is optionally substituted with R₄, C₁ alkyl-R₄, C₂₋₆alkenyl-R₄, or C₂₋₆alkynyl-R₄ which in each case may be further optionally substituted. Preferably R₄ is a C₆₋₁₀aryl such as phenyl or a 4-10-membered heterocycle such as 5-membered, 6-membered, 9-membered or 10-membered heterocycles. In a particular embodiment R is optionally substituted with halo, an optionally substituted phenyl, an optionally substituted 5-membered monocyclic heterocycle or an optionally substituted 6-membered heterocycle. In a further embodiment R is substituted with an optionally substituted phenyl, an optionally substituted 5-membered monocyclic heteroaryl including but not limited to pyrrolyl, pyrazolyl, imidazolyl, triazoyl, tetrazoyl, furanyl, oxazolyl, isooxazoyl, oxaziazolyl, thiophenyl, thiazolyl, isothiazoyl and thiadiazolyl or an optionally substituted 6-membered heteroaryl including but not limited to pyridinyl, pyridazinyl, pyrimidinyl and pyrazinyl. R may be further optionally substituted with halo.

In another embodiment, when X is an optionally substituted C₁ alkyl, C₂₋₆alkenyl or C₂₋₆alkynyl, suitable optional substituents include but are not limited to one or more substituents independently selected from hydroxyl, C₁₋₆alkoxyl, CO₂H, OP(═O)(OH)₂, OP(═O)(OR₅)₂, P(═O)(OH)₂, P(═O)(OR₅)₂, R₆, OR₆, CO₂—R₆, R₆, OR₅—CO₂H, OR₅—CO₂R₆, C(═O)—R₆, NHC(═O)—R₆, N(R₅)C(═O)—R₆, NHCO₂—R₆, N(R₅)CO₂—R₆, OC(═O)NH—R₆ or O(C═O)N(R₆)₂; where R₅ is selected from C₁ alkyl, C₂₋₆alkenyl or C₂₋₆alkynyl and R₆ is selected from C₁ alkyl, C₂₋₆alkenyl, C₂₋₆alkynyl, an amino group such as NH₂, NH(R₅), or N(R₅)₂; an alkylamino group such as C₁₋₆alkylamino; a 5-6 membered monocyclic heterocycle or an alkylheterocycle group such as C₁₋₆alkylheterocycle where the heterocycle preferably contains nitrogen; a 9-10 membered bicyclic heterocycle or C₁₋₆alkylheterocycle wherein the heterocycle preferably contains nitrogen; or an aryl group such as phenyl or alkylaryl group such as C₁₋₆alkylphenyl; wherein each optional substituent having an available substitutable position may be further optionally substituted.

In one embodiment X is an optionally substituted C₁₋₆alkyl preferably an optionally substituted methyl, ethyl or propyl, even more preferably methyl. In a further embodiment, C₁₋₆alkyl is unsubstituted.

In a particular embodiment the method comprises the administration of a compound of formula (Ib):

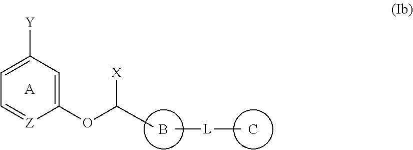

or a pharmaceutically acceptable salt, racemate, isomer, diastereoisomer, enantiomer, hydrate, solvate, N-oxide, pharmaceutically acceptable derivative or prodrug thereof wherein

B ring and C ring are each independently an optionally substituted phenyl, an optionally substituted 5-membered monocyclic heterocycle or an optionally substituted 6-membered heterocycle;

L represents a covalent bond or a C₁₋₆alkylene, C₂₋₆alkenylene or C₂₋₆alkynylene moiety joining B ring and C ring; and

Y, A, Z and X are as previously defined for formula (I).

In a preferred embodiment, B ring is an optionally substituted 5-membered heteroaryl.

In another preferred embodiment C ring is an optionally substituted phenyl.

In still another preferred embodiment L is a covalent bond.

In a further embodiment the method comprises the administration of a compound of formula (Ic):

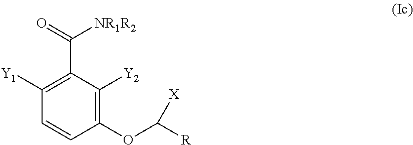

or a pharmaceutically acceptable salt, racemate, isomer, diastereoisomer, enantiomer, hydrate, solvate, N-oxide, pharmaceutically acceptable derivative or prodrug thereof,

wherein Y₁ and Y₂ are independently selected from H, Cl, Br, I and F; R₁ and R₂ are each independently selected from H or optionally substituted C₁₋₆alkyl, preferably H; X is H or optionally substituted C₁₋₆alkyl and R is as previously defined according to formula (I) or is a α-L-C moiety defined according to formula (Ib).

In a further embodiment the method comprises the administration of a compound of formula (Id):

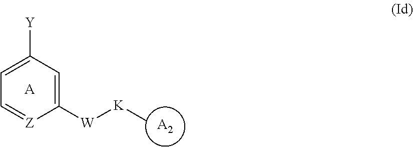

or a salt, racemate, isomer, diastereoisomer, enantiomer, hydrate, solvate, N-oxide, pharmaceutically acceptable derivative or prodrug thereof, wherein

A ring is optionally substituted with one or more substituents;

Y is selected from CONR₁R₂ and C(═NR₃)NR₁R₂ where R₁ and R₂ are independently selected from H or optionally substituted C₁ alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;

Z is C or N;

W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;

K contains an electrophilic carbonyl group and is an optionally substituted linker selected from C₁₋₁₂alkyl, C₂₋₁₂alkenyl, C₂₋₁₂alkynyl, C₃₋₈cycloalkyl, C₆₋₁₀aryl and 4-10-membered heterocyclyl.

A₂ is optionally substituted and is selected from C₆₋₁₀aryl and 5-10-membered heterocycles.

Compounds of Formula (Id) contain a linker K that bears an electrophilic carbonyl substituent. Without limitation by the theory, certain compounds of Formula (Id) may exhibit improved inhibitory properties of C. difficile that arise from a spatial arrangement wherein the proximity of the inhibitor molecule to a serine residue in the binding site permits the formation of a covalent hemiketal adduct.

In one embodiment the Clostridium difficile being treated is a drug resistant Clostridium difficile. In one embodiment the Clostridium difficile is Clostridium difficile (Isolate ID BI-9) and Clostridium difficile (Isolate ID 027-01).

The invention also provides methods for treating Clostridium difficile as described herein wherein the treatment is associated with less or lower disruption or suppression of normal bowel flora when compared to the administration of other antibacterials such as antibacterials used clinically including antibacterials used to treat Clostridium difficile (e.g., metronidazole and/or vancomycin).

### Compounds

The present inventors have also discovered a novel class of compounds per se with demonstrated activity against Clostridium difficile. Accordingly, in one embodiment there is provided a compound of formula (Ia)

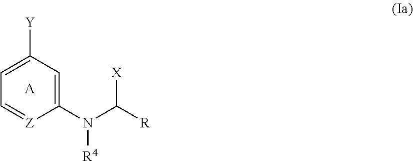

or a salt, racemate, isomer, diastereoisomer, enantiomer, hydrate, solvate, N-oxide, pharmaceutically acceptable derivative or prodrug thereof,

wherein A, X, Y, Z, R and R⁴ are as previously defined.

The introduction of a non-hydrogen X group provides a chiral centre and accordingly, enantiomeric forms of the compounds of Formula (Ia).

In one embodiment there is provided an R-enantiomer of a compound of Formula (Ia) or a salt, hydrate, solvate, N-oxide, pharmaceutically acceptable derivative or prodrug thereof.

In another embodiment there is provided an S-enantiomer of a compound of Formula (Ia) or a salt, hydrate, solvate, N-oxide, pharmaceutically acceptable derivative or prodrug thereof.

In still another embodiment there is provided a mixture of R- and S-enantiomers of a compound of Formula (Ia) or a salt, hydrate, solvate, N-oxide, pharmaceutically acceptable derivative or prodrug thereof.

In another embodiment there is provided a compound of formula (II)

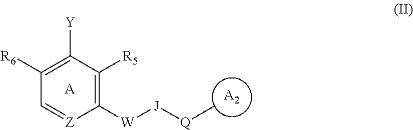

or a salt, racemate, isomer, diastereoisomer, enantiomer, hydrate, solvate, N-oxide, pharmaceutically acceptable derivative or prodrug thereof, wherein A, A₂, Z, Y, W, Q, J, R₆ and R₅ are as previously defined.

In another embodiment, there is provided a compound of formula (II) or a salt, racemate, isomer, diastereoisomer, enantiomer, hydrate, solvate, N-oxide, pharmaceutically acceptable derivative or prodrug thereof,

wherein

R₆ is selected from CI or F.

In a preferred form, when there are one or more substituents there are one, two, three or four substituents or a range between any two of these integers.

In one embodiment, there is provided a compound selected from the group consisting of the compounds in Table 2 or Table 3 or a salt, racemate, isomer, diastereoisomer, enantiomer, hydrate, solvate, N-oxide, pharmaceutically acceptable derivative or prodrug thereof.

The following embodiments are further provided. It is to be understood that these embodiments are for any Formula provided herein (e.g., Formula I″, I, II etc.) It is to be understood that two or more embodiments may be combined.

In one embodiment the A ring is optionally substituted with one, two or three substituents independently selected from halo, hydroxyl, C₁₋₃alkyl, C₂₋₃alkenyl, C₂₋₃alkynyl, C₁₋₃alkoxyl, C₁₋₃alkylhalo, C₁₋₃alkoxylhalo, CN, NH₂, NH(C₁₋₃alkyl), N(C₁₋₃alkyl)₂ and NO₂.

In one embodiment the A ring is optionally substituted with one, two or three substituents independently selected from halo.

In one embodiment the A ring is optionally substituted with one, two or three substituents independently selected from halo and C₁₋₃alkyl.

In one embodiment the A ring is optionally substituted with one, two or three substituents independently selected from halo and methyl.

In one embodiment the A ring is substituted at a position adjacent to the Y group with a halo and optionally substituted with one additional substituents independently selected from halo and C₁₋₃alkyl.

In one embodiment the A ring is substituted independently at each position adjacent to the Y group with a halo.

In one embodiment the A ring is substituted independently at each position adjacent to the Y group with a fluoro.

In one embodiment the A ring is substituted independently at each position adjacent to the Y group with a fluoro or chloro.

In one embodiment the A ring is substituted independently at each position adjacent to the Y group with a fluoro or chloro.

In one embodiment the A ring is substituted only at the positions shown in the formula.

In one embodiment R₅ is selected from F and Cl; R₆ is H.

In one embodiment R₅ is selected from F and Cl; R₆ is H.

In one embodiment R₅ and R₆ are independently selected from F and Cl.

In one embodiment R₅ is F.

In one embodiment R₆ is F.

In one embodiment R₆ is halo.

In one embodiment R₆ is fluoro.

In one embodiment Y is CONR₁R₂.

In one embodiment R₁ and R₂ are each H.

In one embodiment R₁ and R₂ are each H or C₁₋₆alkyl optionally substituted with NH₂, NHCH₃ or N(CH₃)₂. and R₃ is H or OH.

In one embodiment R₃ is OH.

In one embodiment Y is CONR₁R₂.

In one embodiment Y is CONH₂, C(═N(OH))NH₂ or CONHCH₂NHCH₃.

In one embodiment Y is CONH₂, C(═N(OH))NH₂, C(═N(H)NH₂ or CONHCH₂NHCH₃.

In one embodiment Y is CONH₂.

In one embodiment Z is CH.

In one embodiment Z is N.

In one embodiment W is O.

In one embodiment W is NH.

In one embodiment W is NR₄ where R₄ is H or is optionally substituted C₁-12alkyl.

In one embodiment W is NR₄ where R₄ is H or is a C₁₋₁₂alkyl optionally substituted with one or more groups selected from hydroxyl, nitrile, —CONR^{A}R^{B}, (C₁-C₆)alkoxy, monocyclic heteroaryl and COOR^{A}, wherein the monocyclic heteroaryl is optionally substituted with one or more C₁-C₆alkyl groups and wherein R^{A} and R^{B} are independently hydrogen or a (C₁-C₆)alkyl.

In one embodiment W is NR₄ where R₄ is H, methyl, 2-hydroxyethyl, 3-cyanopropyl, 2-amino-2-oxoethyl, 2-methoxyethyl, 6-methylpyridin-3-yl, —CH₂CO₂H or —CH₂CO₂CH₂CH₃.

In one embodiment W is O or NH.

In one embodiment W is NH.

In one embodiment X is H or optionally substituted C₁₋₁₂alkyl.

In one embodiment X is H or C₁₋₁₂alkyl.

In one embodiment X is H or methyl.

In one embodiment X is H or C₁₋₆alkyl optionally substituted with one or more groups selected from hydroxyl, C₁₋₆alkoxyl, CO₂H, OP(═O)(OH)₂, OP(═O)(OR₈)₂, P(═O)(OH)₂, P(═O)(OR₈)₂, R₉, OR₉, CO₂—R₉, OC(═O)—R₉, OR₈—CO₂H, OR₈—CO₂R₉, C(═O)—R₉, NHC(═O)—R₉, N(R₈)C(═O)—R₉, NHCO₂—R₉, N(R₈)CO₂—R₉, OC(═O)NH—R₉ or O(C═O)N(R₉)₂; where R₈ is selected from C₁₋₆alkyl, C₂₋₆alkenyl or C₂₋₆alkynyl and R₉ is selected from C₁₋₆alkyl, C₂₋₆ alkenyl, C₂₋₆ alkynyl, an amino group such as NH₂, NH(R₈), or N(R₈)₂; an alkylamino group such as C₁₋₆alkylamino; a 5-6 membered monocyclic heterocycle or an alkylheterocycle group such as C₁₋₆alkylheterocycle where the heterocycle preferably contains nitrogen; a 9-10 membered bicyclic heterocycle or C₁₋₆ alkylheterocycle wherein the heterocycle preferably contains nitrogen; or an aryl group such as phenyl or alkylaryl group such as C₁₋₆alkylphenyl; wherein each optional substituent having an available substitutable position may be further optionally substituted.

In one embodiment X is H or C₁ alkyl optionally substituted with one or more groups selected from hydroxyl, C₁₋₆alkoxyl, OP(═O)(OR₈)₂, OC(═O)—R₉, OR₈—CO₂R₉, OC(═O)NH—R₉ or O(C═O)N(R₉)₂; where R₈ is selected from C₁₋₆alkyl, C₂₋₆alkenyl or C₂₋₆alkynyl and R₆ is selected from C₁₋₆alkyl, C₂₋₆alkenyl, C₂₋₆alkynyl, an amino group such as NH₂, NH(R₈), or N(R₈)₂; an alkylamino group such as C₁₋₆alkylamino; a 5-6 membered monocyclic heterocycle or an alkylheterocycle group such as C₁₋₆alkylheterocycle where the heterocycle preferably contains nitrogen; a 9-10 membered bicyclic heterocycle or C₁₋₆alkylheterocycle wherein the heterocycle preferably contains nitrogen; or an aryl group such as phenyl or alkylaryl group such as C₁₋₆alkylphenyl; wherein each optional substituent having an available substitutable position may be further optionally substituted.

In one embodiment X is H or C₁₋₆alkyl optionally substituted with one or more groups selected from hydroxyl, C₁₋₆alkoxyl, CO₂H, OP(═O)(OH)₂, OP(═O)(ORO₈)₂, P(—O)(OH)₂, P(—O)(OR₈)₂, R₉, OR₉, CO₂—R₉, OC(═O)—R₉, OR₈—CO₂H, OR₈—CO₂R₉, C(═O)—R₉, NHC(═O)—R₉, N(R₈)C(═O)—R₉, NHCO₂—R₉, N(R₈)CO₂—R₉, OC(═O)NH—R₉ or O(C═O)N(R₉)₂; where R₈ is selected from C₁₋₆alkyl, C₂₋₆alkenyl or C₂₋₆alkynyl and R₉ is selected from C₁₋₆alkyl, C₂₋₆alkenyl, C₂₋₆alkynyl, an amino group such as NH₂, NH(R₈), or N(R₈)₂; an alkylamino group such as C₁₋₆alkylamino; a 5-6 membered monocyclic heterocycle or an alkylheterocycle group such as C₁₋₆alkylheterocycle where the heterocycle preferably contains nitrogen; a 9-10 membered bicyclic heterocycle or C₁₋₆alkylheterocycle wherein the heterocycle preferably contains nitrogen; or an aryl group such as phenyl or alkylaryl group such as C₁₋₆alkylphenyl; wherein each C₁₋₆alkyl, C₂₋₆alkenyl or C₂₋₆alkynyl of R₈ or R₉ is optionally substituted with one or more groups selected from NH₂, NH(R₈), N(R₈)₂, hydroxyl, C₁₋₆alkoxyl, CO₂H, OP(═O)(OH)₂, OP(═O)(OR₈)₂, P(═O)(OH)₂, P(═O)(OR₈)₂, R₉, OR₉, CO₂—R₉, OC(═O)—R₉, OR₈—CO₂H, OR₈—CO₂R₉, C(═O)—R₉, NHC(═O)—R₉, N(R₈)C(═O)—R₉, NHCO₂—R₉, N(R₈)CO₂—R₉, OC(═O)NH—R₉ or O(C═O)N(R₉)₂, 5-6 membered monocyclic heterocycle and 9-10 membered bicyclic heterocycle wherein the 5-6 membered monocyclic heterocycle and 9-10 membered bicyclic heterocycle is optionally substituted with one or more C₁₋₆alkyl, oxo or C₁₋₆alkoxy.

In one embodiment X is H or C₁₋₆alkyl optionally substituted with one or more groups selected from hydroxyl, C₁₋₆alkoxyl, OP(═O)(OH)₂, OP(═O)(OR₈)₂, OC(═O)—R₉, OR₈—CO₂R₉, C(═O)—R₉, NHC(═O)—R₉, N(R₈)C(═O)—R₉, NHCO₂—R₉, N(R₈)CO₂—R₉, OC(═O)NH—R₉ or O(C═O)N(R₉)₂; where R₈ is selected from C₁₋₆alkyl, C₂₋₆alkenyl or C₂₋₆alkynyl and R₉ is selected from C₁₋₆alkyl, C₂₋₆alkenyl, C₂₋₆alkynyl, an amino group such as NH₂, NH(R₈), or N(R₈)₂; an alkylamino group such as C₁₋₆alkylamino; a 5-6 membered monocyclic heterocycle or an alkylheterocycle group such as C₁₋₆ alkylheterocycle where the heterocycle preferably contains nitrogen; a 9-10 membered bicyclic heterocycle or C₁₋₆alkylheterocycle wherein the heterocycle preferably contains nitrogen; or an aryl group such as phenyl or alkylaryl group such as C₁₋₆alkylphenyl; wherein each C₁₋₆alkyl, C₂₋₆alkenyl or C₂₋₆alkynyl of R₈ or R₉ is optionally substituted with one or more groups selected from NH₂, NH(R₈), N(R₈)₂, hydroxyl, C₁₋₆alkoxyl, CO₂H, OP(═O)(OH)₂, OP(═O)(OR₈)₂, P(═O)(OH)₂, P(═O)(OR₈)₂, R₉, OR₉, CO₂—R₉, OC(═O)—R₉, OR₈—CO₂H, OR₈—CO₂R₉, C(═O)—R₉, NHC(═O)—R₉, N(R₈)C(═O)—R₉, NHCO₂—R₉, N(R₈)CO₂—R₉, OC(═O)NH—R₉ or O(C═O)N(R₉)₂, 5-6 membered monocyclic heterocycle and 9-10 membered bicyclic heterocycle wherein the 5-6 membered monocyclic heterocycle and 9-10 membered bicyclic heterocycle is optionally substituted with one or more C₁₋₆alkyl, oxo or C₁₋₆alkoxy.

In one embodiment X is H or C₁₋₆alkyl optionally substituted with one or more hydroxyl.

In one embodiment X is H or C₁₋₆alkyl.

In one embodiment X is H methyl or hydroxylmethyl.

In one embodiment R is optionally substituted and is selected from C₁₋₁₂alkyl, C₂₋₁₂alkenyl, C₂₋₁₂alkynyl, C₆₋₁₀aryl and 4-10-membered heterocycles.

In one embodiment R is optionally substituted and is selected from C₁₋₁₂alkyl, phenyl, naphthyl, a 5-membered monocyclic heterocycle, a 6-membered monocyclic heterocycle, a 9-membered bicyclic heterocycle and a 10-membered bicyclic heterocycle.

In one embodiment R is optionally substituted and is selected from phenyl and 5-6 membered monocyclic heteroaryl.

In one embodiment R is optionally substituted and is 5-6 membered monocyclic heteroaryl, 9-membered bicyclic heteroaryl or a 10-membered bicyclic heteroaryl.

In one embodiment R is optionally substituted and is selected from C₂₋₁₂alkyl, C₂₋₁₂alkenyl, C₂₋₁₂alkynyl, thiazolyl, oxadiazolyl, oxazolyl, thiadiazolyl, pyrazolyl, thienyl, pyrimidinyl, pyridinyl, triazolyl, benzothiaxolyl and thiazolo[5,4-b]pyridine.

In one embodiment R is optionally substituted with one or more substituents independently selected from halo, hydroxyl, NO₂, CN, C₁₋₆alkyl, C₂₋₆alkenyl, C₂₋₆alkynyl, C₁₋₆alkoxy, haloC₁₋₆alkyl, haloC₁₋₆alkoxy, NH₂, NH(C₁₋₆alkyl), N(C₁₋₆alkyl)₂, R₄, C₁₋₆alkyl-R₄, C₂₋₆alkenyl-R₄, C₂₋₆alkynyl-R₄ where R₄ is selected from C₃₋₈cycloalkyl, C₆₋₁₀aryl and 4-10-membered heterocycles wherein each optional substituent having an available substitutable position may be further optionally substituted with one or more groups selected from halo, hydroxyl, SF₅, NO₂, CN, C₁₋₆alkyl, C₂₋₆alkenyl, C₂₋₆alkynyl, C₁₋₆alkoxy, haloC₁₋₆alkyl, haloC₁₋₆alkoxy, NH₂, NH(C₁₋₆alkyl), N(C₁₋₆alkyl)₂, R₄, C₁₋₆alkyl-R₄, C₂₋₆alkenyl-R₄, C₂₋₆alkynyl-R₄ where R₄ is selected from C₃₋₈cycloalkyl, C₆₋₁₀aryl or 4-10-membered heterocycles wherein C₆₋₁₀aryl is optionally substituted with one or more C₁₋₆alkyl or SF₅.

In one embodiment R is optionally substituted with one or more substituents independently selected from halo, C₁₋₆alkyl, C₂₋₆alkenyl, C₁₋₆alkoxy, R₄, C₁₋₆alkyl-R₄, where R₄ is selected from C₆₋₁₀aryl and 4-10-membered heterocycles wherein each optional substituent having an available substitutable position may be further optionally substituted with one or more groups selected from halo, SF₅, C₁₋₆alkoxy, C₁₋₆alkyl, haloC₁₋₆alkyl, haloC₁₋₆alkoxy, R₄, where R₄ is selected from C₆₋₁₀aryl optionally substituted with one or more C₁₋₆alkyl or SF₅.

In one embodiment R is optionally substituted with one or more substituents independently selected from halo, C₁₋₆alkyl, haloC₁₋₆alkyl, C₂₋₆alkenyl, C₂₋₆alkynyl, C₁₋₆alkoxy, —SC₁₋₆alkyl, R₄, C₁₋₆alkyl-R₄, where R₄ is selected from phenyl, pyrimidinyl, oxazolyl, oxadiazolyl, thienyl, thiazolyl, and benzothiazolyl wherein each optional substituent having an available substitutable position may be further optionally substituted with one or more groups selected from halo, hydroxyl, SF₅, C₁₋₆alkoxy, C₁₋₆alkyl, haloC₁₋₆alkyl, haloC₁₋₆alkoxy, CO₂C₁₋₆alkyl, and phenyl optionally substituted with one or more halo, C₁₋₆alkyl, haloC₁₋₆alkoxy or SF₅.

In one embodiment R is optionally substituted with one or more substituents independently selected from halo, C₁₋₆alkyl, haloC₁₋₆alkyl, C₂₋₆alkenyl, C₂₋₆alkynyl, C₁₋₆ alkoxy, R₄, C₁₋₆alkyl-R₄, where R₄ is selected from phenyl, pyrimidinyl, oxazolyl, oxadiazolyl, thienyl, thiazolyl, and benzothiazolyl wherein each optional substituent having an available substitutable position may be further optionally substituted with one or more groups selected from halo, hydroxyl, SF₅, C₁₋₆alkoxy, C₁₋₆alkyl, haloC₁₋₆ alkyl, haloC₁₋₆alkoxy, CO₂C₁₋₆alkyl, and phenyl optionally substituted with one or more halo, C₁₋₆alkyl, haloC₁₋₆alkoxy or SF₅.

In one embodiment R is selected from C₂₋₁₂alkyl, C₂₋₁₂alkenyl, C₂₋₁₂alkynyl, thiazolyl, oxadiazolyl, oxazolyl, thiadiazolyl, pyrazolyl, thienyl, pyrimidinyl, pyridinyl, triazolyl, benzothiaxolyl and thiazolo[5,4-b]pyridine each of which is optionally substituted with one or more substituents independently selected from halo, C₁₋₆alkyl, haloC₁₋₆alkyl, C₂₋₆alkenyl, C₂₋₆alkynyl, C₁₋₆alkoxy, R₄, C₁₋₆alkyl-R₄, where R₄ is selected from phenyl, pyrimidinyl, oxazolyl, oxadiazolyl, thienyl, thiazolyl, and benzothiazolyl wherein each optional substituent having an available substitutable position may be further optionally substituted with one or more groups selected from halo, hydroxyl, SF₅, C₁₋₆alkoxy, C₁₋₆alkyl, haloC₁₋₆alkyl, haloC₁₋₆alkoxy, CO₂C₁₋₆alkyl, and phenyl optionally substituted with one or more halo, C₁₋₆alkyl, haloC₁₋₆alkoxy or SF₅.

In one embodiment R is selected from C₁₋₁₂alkyl, phenyl, a 5-membered monocyclic heterocycle, a 6-membered monocyclic heterocycle, a 9-membered bicyclic heterocycle and a 10-membered bicyclic heterocycle each of which is optionally substituted with one or more substituents independently selected from halo, C₁₋₆alkyl, haloC₁₋₆alkyl, C₂₋₆alkenyl, C₂₋₆alkynyl, C₁₋₆alkoxy, R₄, C₁₋₆alkyl-R₄, where R₄ is selected from phenyl, pyrimidinyl, oxazolyl, oxadiazolyl, thienyl, thiazolyl, and benzothiazolyl wherein each optional substituent having an available substitutable position may be further optionally substituted with one or more groups selected from halo, hydroxyl, SF₅, C₁₋₆alkoxy, C₁₋₆alkyl, haloC₁₋₆alkyl, haloC₁₋₆alkoxy, CO₂C₁₋₆alkyl, and phenyl optionally substituted with one or more halo, C₁₋₆alkyl, haloC₁₋₆alkoxy or SF₅.

In one embodiment R is optionally substituted with one or more substituents independently selected from halo, hydroxyl, NO₂, CN, C₁₋₆alkyl, C₂₋₆alkenyl, C₂₋₆alkynyl, C₁₋₆alkoxy, haloC₁₋₆alkyl, haloC₁₋₆alkoxy, NH₂, NH(C₁₋₆alkyl), N(C₁₋₆alkyl)₂, R₄, C₁₋₆alkyl-R₄, C₂₋₆alkenyl-R₄, C₂₋₆alkynyl-R₄ where R₄ is selected from C₃₋₈cycloalkyl, C₆₋₁₀aryl and 4-10-membered heterocycles wherein each optional substituent having an available substitutable position may be further optionally substituted with one or more groups selected from halo, hydroxyl, NO₂, CN, C₁₋₆alkyl, C₂₋₆alkenyl, C₂₋₆alkynyl, C₁₋₆alkoxy, haloC₁₋₆alkyl, haloC₁₋₆alkoxy, NH₂, NH(C₁₋₆alkyl), N(C₁₋₆alkyl)₂, R₄, C₁₋₆alkyl-R₄, C₂₋₆alkenyl-R₄, C₂₋₆alkynyl-R₄ where R₄ is selected from C₃₋₈cycloalkyl, C₆₋₁₀aryl or 4-10-membered heterocycles wherein C₆₋₁₀aryl is optionally substituted with one or more C₁₋₆alkyl.

In one embodiment R is optionally substituted with one or more substituents independently selected from halo, C₁₋₆alkyl, C₂₋₆alkenyl, C₁₋₆alkoxy, R₄, C₁₋₆alkyl-R₄, where R₄ is selected from C₆₋₁₀aryl and 4-10-membered heterocycles wherein each optional substituent having an available substitutable position may be further optionally substituted with one or more groups selected from halo, C₁₋₆alkoxy, C₁₋₆alkyl, haloC₁₋₆alkyl, haloC₁₋₆alkoxy, R₄, where R₄ is selected from C₆₋₁₀aryl optionally substituted with one or more C₁₋₆alkyl.

In one embodiment R is an optionally substituted with (C₁-C₆)alkyl, (C₂-C₆)alkenyl, (C₂-C₆)alkynyl, (C₁-C₆)alkoxy, hydroxy, hydroxy(C₁-C₆)alkyl, (C₁-C₃)alkoxy(C₁-C₃)alkyl, mercapto, mercapto(C₁-C₆)alkyl, (C₁-C₆)alkylthio, halo, fully or partially fluorinated (C₁-C₃)alkyl, (C₁-C₃)alkoxy or (C₁-C₃)alkylthio, nitro, nitrile (—CN), oxo (═O), thiols, alkylthiols, trialkylsilyl, diarylalkylsilyl, trialkylsilyloxy, diarylalkylsilyloxy, dialkylphosphonyl, dialkoxyphosphonyl, diarylphosphonyl, diaryloxyphosphonyl, alkylphosphinyl, arylphosphinyl, alkoxyphosphinyl, aryloxyphosphinyl, dialkyoxyphoshoryl, diaryloxyphosphoryl, phosphoryl, phosphinyl, phenyl, phenyl(C₁-C₃)alkyl-, phenoxy, monocyclic heteroaryl, heteroaryl(C₁-C₃)alkyl-, or heteroaryloxy with 5 or 6 ring atoms, cycloalkyl having 3 to 6 ring carbon atoms, —COOR^{A}, —COR^{A}, —OCOR^{A}, —SO₂R^{A}, —CONR^{A}R^{B}, —CONHNH₂, —S₂NR^{A}R^{B}, SF₅, —NR^{A}R^{B}, —NHNH₂, —OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl, hydroxy(C₁-C₆)alkyl, or (C₁-C₃)alkoxy(C₁-C₃)alkyl group or, in the case where R^{A} and R^{B} are linked to the same N atom, R^{A} and R^{B} taken together with that nitrogen may form a cyclic amino ring such as morpholinyl, piperidinyl, piperazinyl, or 4-(C₁-C₆)alkyl-piperizinyl, wherein each optional alkyl, cycloalkyl, alkylaryl, aryl, heterocyclyl, or heteroaryl substituent may also be optionally substituted.

In one embodiment R is an optionally substituted with (C₁-C₆)alkyl, (C₂-C₆)alkenyl, (C₁-C₆)alkoxy, halo, phenyl, phenyl(C₁-C₃)alkyl-, phenoxy, monocyclic heteroaryl, heteroaryl(C₁-C₃)alkyl-, or heteroaryloxy with 5 or 6 ring atoms, wherein each optional alkyl, alkylaryl, aryl, heterocyclyl, or heteroaryl substituent may also be optionally substituted.

In one embodiment R is optionally substituted and is selected from C₁₋₁₂alkyl, and 4-10-membered heterocycles.

In one embodiment R is optionally substituted and is selected from C₁₋₁₂alkyl, and 5-membered heteroaryl.

In one embodiment R is an optionally substituted with (C₁-C₆)alkyl, (C₂-C₆)alkenyl, (C₂-C₆)alkynyl, (C₁-C₆)alkoxy, hydroxy, hydroxy(C₁-C₆)alkyl, (C₁₋C₃)alkoxy(C₁₋₃)alkyl, mercapto, mercapto(C₁-C₆)alkyl, (C₁-C₆)alkylthio, halo, fully or partially fluorinated (C₁-C₃)alkyl, (C₁-C₃)alkoxy or (C₁-C₃)alkylthio, nitro, nitrile (—CN), oxo (═O), thiols, alkylthiols, trialkylsilyl, diarylalkylsilyl, trialkylsilyloxy, diarylalkylsilyloxy, dialkylphosphonyl, dialkoxyphosphonyl, diarylphosphonyl, diaryloxyphosphonyl, alkylphosphinyl, arylphosphinyl, alkoxyphosphinyl, aryloxyphosphinyl, dialkyoxyphoshoryl, diaryloxyphosphoryl, phosphoryl, phosphinyl, phenyl, phenyl(C₁-C₃)alkyl-, phenoxy, monocyclic heteroaryl, heteroaryl(C₁-C₃)alkyl-, or heteroaryloxy with 5 or 6 ring atoms, cycloalkyl having 3 to 6 ring carbon atoms, —COOR^{A}, —COR^{A}, —OCOR^{A}, —SO₂R^{A}, —CONR^{A}R^{B}, —CONHNH₂, —SO₂NR^{A}R^{B}, SF₅, —NR^{A}R^{B}, —NHNH₂, —OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl, hydroxy(C₁-C₆)alkyl, or (C₁-C₃)alkoxy(C₁-C₃)alkyl group or, in the case where R^{A} and R^{B} are linked to the same N atom, R^{A} and R^{B} taken together with that nitrogen may form a cyclic amino ring such as morpholinyl, piperidinyl, piperazinyl, or 4-(C₁-C₆)alkyl-piperizinyl, wherein each optional substituent may also be optionally substituted.

In one embodiment R is an optionally substituted with one or more halo, phenyl, phenoxy, or heteroaryloxy with 5 or 6 ring atoms, wherein each phenoxy, phenoxy or heteroaryloxy is optionally substituted with one or more fully or partially fluorinated (C₁-C₃)alkyl or fully or partially fluorinated (C₁-C₃)alkoxy.

In one embodiment a compound of Formula Ia is a compound of compound number 122, 123, 150, 152, 179-187 or 212 or a salt, racemate, isomer, diastereoisomer, enantiomer, hydrate, solvate, N-oxide, pharmaceutically acceptable derivative or prodrug thereof.

In one embodiment a compound of Formula Ia is a compound of compound number 122 or 123.

In one embodiment Q is selected from O or NR₇ where R₇ is H.

In one embodiment Q is O.

In one embodiment Q is CH₂.

In one embodiment Q is selected from O, NH or CH₂.

In one embodiment J is an optionally substituted linker selected from C₁₋₁₂alkyl, C₂₋₁₂alkenyl and C₂₋₁₂alkynyl, wherein the linker is interrupted by an ether linkage.

In one embodiment J is a linker selected from C₁₋₁₂alkyl, C₂₋₁₂alkenyl, C₂₋₁₂alkynyl, interrupted by an ether linkage.

In one embodiment J is an optionally substituted C₁₋₁₂alkyl or C₂₋₁₂alkynyl.

In one embodiment J is C₁₋₁₂alkyl, interrupted by an ether linkage.

In one embodiment J is C₁₋₁₂alkyl or C₂₋₁₂alkynyl each interrupted by an ether linkage.

In one embodiment J is C₁₋₁₂alkyl or C₂₋₁₂alkynyl.

In one embodiment J is an optionally substituted linker selected from C₁₋₁₂alkyl and C₂₋₁₂alkynyl.

In one embodiment J is C₁₋₁₂alkyl, optionally interrupted by an ether linkage.

In one embodiment A₂ is an optionally substituted C₆₋₁₀aryl.

In one embodiment A₂ is an optionally substituted phenyl.

In one embodiment A₂ is an optionally substituted 5-10-membered heterocycle.

In one embodiment A₂ is an optionally substituted phenyl, pyridinyl or pyrimidinyl.

In one embodiment A₂ is an optionally substituted with (C₁-C₆)alkyl, (C₂-C₆)alkenyl, (C₂-C₆)alkynyl, (C₁-C₆)alkoxy, hydroxy, hydroxy(C₁-C₆)alkyl, (C₁-C₃)alkoxy(C₁-C₃)alkyl, mercapto, mercapto(C₁-C₆)alkyl, (C₁-C₆)alkylthio, halo, fully or partially fluorinated (C₁-C₃)alkyl, (C₁-C₃)alkoxy or (C₁-C₃)alkylthio, nitro, nitrile (—CN), oxo (═O), thiols, alkylthiols, trialkylsilyl, diarylalkylsilyl, trialkylsilyloxy, diarylalkylsilyloxy, dialkylphosphonyl, dialkoxyphosphonyl, diarylphosphonyl, diaryloxyphosphonyl, alkylphosphinyl, arylphosphinyl, alkoxyphosphinyl, aryloxyphosphinyl, dialkyoxyphoshoryl, diaryloxyphosphoryl, phosphoryl, phosphinyl, phenyl, phenyl(C₁-C₃)alkyl-, phenoxy, monocyclic heteroaryl, heteroaryl(C₁-C₃)alkyl-, or heteroaryloxy with 5 or 6 ring atoms, cycloalkyl having 3 to 6 ring carbon atoms, —COOR^{A}, —COR^{A}, —OCOR^{A}, —SO₂R^{A}, —CONR^{A}R^{B}, —CONHNH₂, —SO₂NR^{A}R^{B}, SF₅, —NR^{A}R^{B}, —NHNH₂, —OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl, hydroxy(C₁-C₆)alkyl, or (C₁-C₃)alkoxy(C₁-C₃)alkyl group or, in the case where R^{A} and R^{B} are linked to the same N atom, R^{A} and R^{B} taken together with that nitrogen may form a cyclic amino ring such as morpholinyl, piperidinyl, piperazinyl, or 4-(C₁-C₆)alkyl-piperizinyl.

In one embodiment A₂ is an optionally substituted with (C₁-C₆)alkyl, (C₁₋C₆)alkoxy, halo, —CN, fully or partially fluorinated (C₁-C₃)alkyl, (C₁-C₃)alkoxy or (C₁-C₃)alkylthio, or SF₅.

In one embodiment A₂ is an optionally substituted with halo, —CN, fully or partially fluorinated (C₁-C₃)alkyl, fully or partially fluorinated (C₁-C₃)alkoxy or SF₅.

In one embodiment A₂ is an optionally substituted with halo, fully or partially fluorinated (C₁-C₃)alkyl, fully or partially fluorinated (C₁-C₃)alkoxy or SF₅.

In one embodiment a compound of Formula Ia is a compound of compound number 124-187 or a salt, racemate, isomer, diastereoisomer, enantiomer, hydrate, solvate, N-oxide, pharmaceutically acceptable derivative or prodrug thereof.

In one embodiment a compound of Formula Ia is a compound of compound number 136, 145, 146, 156 or 161 or a salt, racemate, isomer, diastereoisomer, enantiomer, hydrate, solvate, N-oxide, pharmaceutically acceptable derivative or prodrug thereof.

### Definitions

As used herein, the term “halo” or “halogen” refers to fluorine (fluoro), chlorine (chloro), bromine (bromo) or iodine (iodo).

As used herein, the term “alkyl” either used alone or in compound terms such as NH(alkyl) or N(alkyl)₂, refers to monovalent straight chain or branched hydrocarbon groups, having 1 to 3, 1 to 6 or 1 to 12 carbons as appropriate. Each C₁₋₆alkyl group is preferably C₁, C₂ or C₃ alkyl, i.e. C₁₋₃alkyl. For example, suitable alkyl groups include, but are not limited to methyl, ethyl, propyl, isopropyl, n-butyl, sec-butyl, tert-butyl, pentyl, 2-methylbutyl, 3-methylbutyl, n-hexyl, 2-, 3- or 4-methylpentyl, 2-ethylbutyl, n-hexyl or 2-, 3-, 4- or 5-methylpentyl.

The term “haloalkyl” refers to an alkyl group which has one or more halo substituents. One, two or three halo substituents are particularly preferred. For instance, CF₃ is a haloalkyl group as is CHF₂.

As used herein, the term “alkenyl” refers to a straight chain or branched hydrocarbon groups having one or more double bonds between carbon atoms. Suitable alkenyl groups include, but are not limited to, ethenyl, allyl, propenyl, iso-propenyl, butenyl, pentenyl and hexenyl. Each C₂₋₆alkynyl group is preferably C₂ or C₃ alkynyl, i.e. C₂₋₃alkynyl.

As used herein, the term “alkynyl” refers to a straight chain or branched hydrocarbon groups having one or more triple bonds between carbon atoms. Each C₂₋₆alkenyl group is preferably C₂ or C₃ alkyl, ie C₂₋₃alkyl.

The terms “cycloalkyl”, “carbocyclic” and “carbocyclyl” as used herein, refers to cyclic hydrocarbon groups. Suitable cycloalkyl groups include, but are not limited to cyclopropyl, cyclobutyl, cyclopentyl and cyclohexyl.

The term “aryl” as used herein, refers to a C₆-C₁₀ aromatic hydrocarbon group, for example phenyl or naphthyl.

The term “alkylaryl” includes, for example, benzyl.

The terms “heterocycle”, “heterocyclic” and “heterocyclyl” when used alone or in compound words includes monocyclic, polycyclic, fused or conjugated hydrocarbon residues wherein one or more carbon atoms (and where appropriate, hydrogen atoms attached thereto) are replaced by a heteroatom so as to provide a non-aromatic residue. The bonds between atoms may be saturated or unsaturated. Suitable heteroatoms include O, N and S. Where two or more carbon atoms are replaced, this may be by two or more of the same heteroatom or by different heteroatoms. Suitable examples of heterocyclic groups may include azetidine, pyrrolidinyl, piperidyl, piperazinyl, azepane, morpholino, quinolinyl, isoquinolinyl, thiomorpholino, dioxanyl, 2,2′-dimethyl-[1,3]-dioxolanyl, tetrahydrofuranyl, tetrahydropyranyl, tetrahydropyrrolyl, cyclic sulfonamides such as sultams etc. The term heterocyclyl will be understood to encompass heteroaromatic/heteroaryl ring systems.

The term “heteroaromatic” or “heteroaryl” may be used interchangeably and includes but is not limited to a 5- or 6-membered heteroaromatic ring containing one or more heteroatoms selected from O, N and S. Suitable examples of heteroaryl groups include 5-membered heteroaryls such as furanyl, thiophenyl, tetrazolyl, 1,2,3-triazolyl, 1,2,4-triazolyl, imidazolyl, pyrrolyl, pyrazolyl, oxazolyl, isoxazolyl, oxadiazolyl, thioazolyl, isothiazolyl, thiodiazolyl, etc and 6-membered heteroaryls such as pyridinyl, pyrimidinyl, pyrazinyl, pyridazinyl, etc. The heteroaromatic ring may be fused to a 5- or 6-membered aromatic or heteroaromatic ring to form an 8-10 membered bicyclic aromatic ring system eg benzofuran, pyrrolopyrimidine, furopyridine, benzothiazole, benzisothiazole, benzoxazole, benzisoxazole, benzimidazole, benztriazole, benzothiophene, oxazolopyridine, imidazopyridine, thiazolopyridine, quinoline, isoquinoline, indazole, indole, isoindole, etc.

The term “leaving group” will be understood by the skilled person and means a molecular fragment which is capable of being displaced as a stable species taking it with it the bonding electrons. Leaving groups are used in organic chemistry to facilitate covalent bonding between two moieties. The term “leaving group” includes but is not limited to, halo groups (such as iodo, bromo, and chloro) or sulfonate ester groups such as mesylate, tosylate, osylate, nosylate, or besylate.

Unless otherwise stated, each alkyl, cycloalkyl, alkylaryl, aryl, heterocyclyl, or heteroaryl group may be optionally substituted with, for example, (C₁-C₆)alkyl, (C₂-C₆)alkenyl, (C₂-C₆)alkynyl, (C₁-C₆)alkoxy, hydroxy, hydroxy(C₁-C₆)alkyl, (C₁₋C₃)alkoxy(C₁-C₃)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo (═O), thiols, alkylthiols, trialkylsilyl, diarylalkylsilyl, trialkylsilyloxy, diarylalkylsilyloxy, dialkylphosphonyl, dialkoxyphosphonyl, diarylphosphonyl, diaryloxyphosphonyl, alkylphosphinyl, arylphosphinyl, alkoxyphosphinyl, aryloxyphosphinyl, dialkyoxyphoshoryl, diaryloxyphosphoryl, phosphoryl, phosphinyl, phenyl, phenyl(C₁-C₃)alkyl-, phenoxy, monocyclic heteroaryl, heteroaryl(C₁-C₃)alkyl-, or heteroaryloxy with 5 or 6 ring atoms, cycloalkyl having 3 to 6 ring carbon atoms, —COOR^{A}, —COR^{A}, —OCOR^{A}, —SO₂R^{A}, —CONR^{A}R^{B}, —CONHNH₂, —SO₂NR^{A}R^{B}, SF₅, —NR^{A}R^{B}, —NHNH₂, —OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl, hydroxy(C₁-C₆)alkyl, or (C₁-C₃)alkoxy(C₁-C₃)alkyl group or, in the case where R^{A} and R^{B} are linked to the same N atom, R^{A} and R^{B} taken together with that nitrogen may form a cyclic amino ring such as morpholinyl, piperidinyl, piperazinyl, or 4-(C₁-C₆)alkyl-piperizinyl such as 4-methyl-piperazinyl. In a preferred form, each alkyl, cycloalkyl, alkylaryl, aryl, heterocyclyl, or heteroaryl group may be optionally substituted with one or more of C₁-C₃alkyl, C₃-C₆cycloalkyl, C₆aryl, heterocyclyl, heteroaryl, C₁-C₃alkylOH, alkylaryl, OH, OC₁-C₃alkyl, halo, CN, NO₂, CO₂H, CO₂C₁-C₃alkyl, CONH₂, CONH(C₁-C₃alkyl), C(O)N(C₁-C₃alkyl)₂, haloC₁₋₃alkyl such as CF₃ and CHF₂, haloC₁₋₃alkoxy such as OCHCF₂ and OCF₃, ═O, SF₅, C(O)C₁₋₃alkyl, C(O)haloC₁₋₃alkyl, NH₂, NH(C₁-C₃alkyl) or N(C₁-C₃alkyl)₂. For example, an optionally substituted aryl group may be 4-methylphenyl or 4-hydroxyphenyl group, and an optionally substituted alkyl group may be 2-hydroxyethyl, trifluoromethyl, or difluoromethyl. Each optional alkyl, cycloalkyl, alkylaryl, aryl, heterocyclyl, or heteroaryl substituent may also be optionally substituted.

In a preferred form, where a group is substituted by an optional substituent, there are 1 to 4 optional substituents, 1 to 3 optional substituents, 1 to 2 optional substituents, or 1 optional substituent.

Examples of optional substituents also include suitable oxygen and nitrogen protecting groups (see “Greene's Protective Groups in Organic Synthesis” Peter G. M. Wuts and Theodora W. Greene, Fourth Edition, Wiley, 2006).

The salts of the compounds are preferably pharmaceutically acceptable, but it will be appreciated that non-pharmaceutically acceptable salts also fall within the scope of the present invention, since these are useful as intermediates in the preparation of pharmaceutically acceptable salts.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1^{st} edition, 2002, Wiley-VCH.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Hydroxyl groups may be esterified with groups including lower alkyl carboxylic acids, such as acetic acid and 2,2-dimethylpropionic acid, or sulfonated with groups including alkyl sulfonic acids, such as methyl sulfonic acid.

It will be recognized that the compounds are likely to possess asymmetric centers (particularly about the carbon of which X or X₁ is a substituent) and are therefore capable of existing in more than one stereoisomeric form. The invention thus also relates to compounds in substantially pure isomeric form at one or more asymmetric centers e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution. In a one embodiment, the stereochemistry around the carbon substituted with X or X₁ is R. In another embodiment, the stereochemistry around the carbon substituted with X or X₁ is S.

This invention also encompasses prodrugs of the compounds. Compounds having free amino, amido, hydroxy or carboxylic groups can be converted into prodrugs.

Prodrugs include compounds wherein an amino acid residue, or a polypeptide chain of two or more (e.g., two, three or four) amino acid residues which are covalently joined to free amino, hydroxy and carboxylic acid groups of the compounds. The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of the compounds through hydroxyl, amine or carbonyl functionalities. Prodrugs also include phosphate derivatives of compounds (such as acids, salts of acids, or esters) joined through a phosphorus-oxygen bond to a free hydroxyl of the compounds.

Other prodrugs include esters or peptides formed respectively between hydroxyl groups or amine groups of the compounds.

### Compositions

The compounds of the present invention may be administered by any suitable means, for example, orally, parenterally, such as by subcutaneous, intravenous, intramuscular, or intracisternal injection or infusion techniques (e.g., as sterile injectable aqueous or non-aqueous solutions or suspensions).

In a preferred embodiment the administration is intravenous administration, oral administration or a combination thereof.

There is also provided a composition comprising a compound of the present invention. Preferably, the composition further comprises a pharmaceutically acceptable carrier, diluent or excipient.

The compositions of the present invention may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Pharmaceutical formulations include those for oral, rectal, nasal, topical (including buccal and sub-lingual), vaginal or parenteral (including intramuscular, subcutaneous and intravenous) administration or in a form suitable for administration by inhalation or insufflation. The compounds of the invention, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, in the form of suppositories for rectal administration; or in the form of sterile injectable solutions for parenteral (including subcutaneous) use.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated.

However, the method can also be practiced in other species, such as avian species (e.g., chickens).

The subjects treated in the above method are mammals, including, but not limited to, cows, sheep, goats, horses, dogs, cats, guinea pigs, rats or other bovine, ovine, equine, canine, feline, rodent or murine species, and preferably a human being, male or female.

The term “effective amount” means the amount of the subject composition that will elicit the biological or medical response of a tissue, system, animal or human that is being sought by the researcher, veterinarian, medical doctor or other clinician.

By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The term “composition” as used herein is intended to encompass a product comprising the specified ingredients in the specified amounts, as well as any product which results, directly or indirectly, from combination of the specified ingredients in the specified amounts.

It is to be understood that a patient can be a mammal such as a human.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In the treatment or prevention of bacterial infections, an appropriate dosage level will generally be about 0.01 to 500 mg per kg patient body weight per day which can be administered in single or multiple doses. For oral administration, the compositions are preferably provided in the form of tablets containing 1.0 to 1000 milligrams of the active ingredient for the symptomatic adjustment of the dosage to the patient to be treated. The compounds may be administered on a regimen of 1 to 4 times per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The pharmaceutical compositions and methods of the present invention may further comprise other therapeutically active compounds which are usually applied in the treatment of bacterial infections. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Accordingly, in one aspect of the present invention, there is provided a pharmaceutical composition comprising a compound of Formula (I″), Formula (I) or Formula (II) and a further antibacterial agent.

In another aspect of the present invention, there is provided a method of treating a bacterial infection comprising administering a compound of Formula (I″), Formula (I) or Formula (II) together with a further antibacterial agent wherein said compound of Formula (I″), Formula (I) or Formula (II) and said antibacterial agent are administered in either order and can be administered simultaneously or sequentially.

Said further antibacterial agent may be selected from the group consisting of those indicated for the treatment of Clostridium difficile infections, including but not limited to for example, vancomycin, metronidazole and fidaxomicin, etc.

When other therapeutic agents are employed in combination with the compounds of the present invention they may be used for example in amounts as noted in the Physician Desk Reference (PDR) or as otherwise determined by one of ordinary skill in the art.

### Methods of Preparation

Compounds of Formula (I) or Formula (II) may generally be prepared by coupling a compound of Formula (III) with a compound of Formula (IV) or (V) under the following conditions

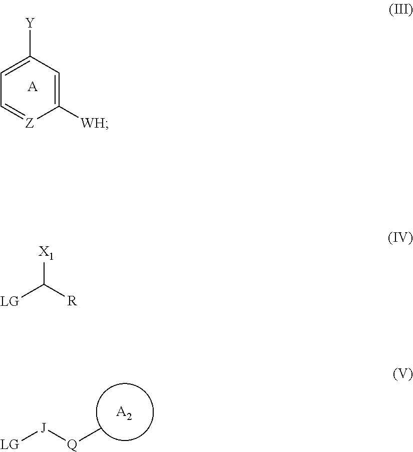

wherein A, A₂, J, Q, X₁, Y, Z and R are as previously defined and LG is a leaving group. To a solution of (IV) or (V) (approximately 1.0 eq) in an organic solvent such as DMF is added a base such as K₂CO₃ (approximately 2.0 eq) followed by (III) (approximately 1.0 eq). The resulting reaction mixture is stirred under a N₂ atmosphere at room temperature. After completion of the reaction the product is extracted into an organic solvent such as EtOAc and purified by silica gel chromatography. Alternatively, where LG is OH, triphenylphosphine (approximately 1.2 eq) is dissolved in a suitable solvent such as THF and treated with diethylazodicarboxylate (approximately 1.2 eq) at 0° C. After stirring for a short time a solution of (III) (1 eq), (IV) or (V) (1 eq) and TEA (1.1 mL, 1 eq) in the same solvent is added to the initial mixture and allowed to warm to room temperature. After completion of the reaction, the mixture may be concentrated and purified by silica chromatography.

It will be understood by those skilled in the art that a considerable diversity of compounds of Formula (I) may be accessed by post-synthetic modification of the R group by means of variation of an α-halo ketone building block. Accordingly, the succeeding methods, which are generally described for the synthesis of compounds of Formula (I), utilise an α-halo ketone as a starting material or precursor intermediate.

An extensive selection of α-bromo and α-chloro ketones is available from commercial suppliers. Alternatively, a further diversity of α-bromo ketones may be prepared according to one or more the following standard methods.

One Pot Conversion of a Substituted Carboxylic Acid to an α-Bromo Ketone

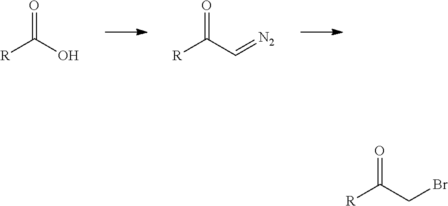

To an ice-cold solution of a substituted carboxylic acid (1.0 eq) in an inert solvent (such as dichloromethane) is added oxalyl chloride (5.0 eq) and DMF (catalytic amount). The resulting reaction mixture is stirred at room temperature. When the reaction is complete, the mixture is concentrated under inert atmosphere to obtain the crude product, which is then dissolved in diethyl ether, cooled to 0° C. and treated with TMS-diazomethane (1.50 eq). The resulting solution is stirred at 0° C. for 30 minutes, followed by addition of HBr (47% aq solution). After completion of reaction, water is added to the mixture and the product extracted into EtOAc (3 times). The combined organics are washed with brine, dried over anhydrous Na₂SO₄, filtered and concentrated in vacuo. The crude residue may be purified by silica chromatography (typically eluted with EtOAc/hexane) to obtain the purified α-bromo ketone, typically in low yield.

### Conversion of a Carboxylic Acid to an α-Bromo Ketone Via a Stille Coupling

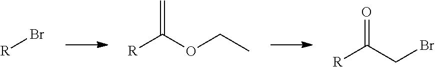

A solution of optionally substituted heteroaryl bromide (1.0 eq) and ethoxyvinyl tri-n-butyltin (1.50 eq) in DMF is purged with nitrogen for 15 minutes followed by addition of tetrakis(triphenylphosphine)palladium (0.10 eq). The resulting solution is again purged with nitrogen for 15 minutes and then heated to approximately 110° C. for 2 h. After the completion of reaction, ice-cold water is added to the reaction mixture, which is then washed with EtOAc (3 times). The combined organics are washed with brine, dried over anhydrous Na₂SO₄, filtered and concentrated in vacuo. The crude residue may be purified by silica chromatography (typically eluted with EtOAc/hexane) to obtain the pure enol ether, typically in good yields. If this product hydrolyses spontaneously to a methyl ketone, it may be converted to the desired α-methyl ketone by one of the methods described below. In all other cases, an ice-cold solution of the enol ether (1.0 eq) in THF-H₂O (3:1) is treated with NBS (1.0 eq) and stirred at room temperature for 30 minutes. After the completion of reaction water is added to the mixture, which is then washed with EtOAc (3 times). The combined organics are washed with brine, dried (Na₂SO₄), filtered and concentrated under reduced pressure. The crude residue may be purified by silica chromatography (typically eluted with EtOAc/hexane) to obtain the purified α-bromo ketone.

Preparation of an α-Bromo Ketone by Bromination of a Methyl Ketone

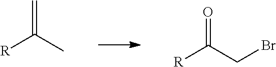

Approach i):

To a solution of a substituted methyl ketone (1.0 eq) in THF is added tetrabutylammonium tribromide (1.0 eq) and the resulting reaction mixture heated at reflux for 2 h. After completion of reaction, water is added to the mixture and the product extracted into EtOAc (3 times). The combined organics are washed with brine, dried (Na₂SO₄), filtered and concentrated under reduced pressure. The crude residue may be purified by silica chromatography (typically eluted with EtOAc/hexane) to obtain the purified α-bromo ketone, typically in good yield.

Approach ii):

A solution of substituted methyl ketone (1.0 eq) in glacial acetic acid is cooled to 0° C. and treated drop-wise with bromine (1.0 eq). A catalytic amount of HBr in acetic acid is added to the reaction mixture and allowed to stir at room temperature typically for 10-20 h. After the completion of reaction, the mixture is cooled to 0° C. and quenched with ice-cold water, followed by extraction with EtOAc (3 times). The combined organics are washed with brine, dried over anhydrous Na₂SO₄, filtered and concentrated in vacuo. The crude residue may be purified by silica chromatography (typically eluted with EtOAc/hexane) to obtain the purified α-bromo ketone, typically in 20-40% yield.

Approach iii):

A solution of substituted methyl ketone (1.0 eq) in THF and 5,5-dibromobarbituric acid (0.90 eq) is heated at reflux, typically for 10-20 h. After completion of the reaction, water is added to the mixture, which is then washed with EtOAc (3 times). The combined organics are washed with brine, dried over anhydrous Na₂SO₄, filtered and concentrated in vacuo. The crude residue may be purified by silica chromatography (typically eluted with EtOAc/hexane) to obtain the purified α-bromo ketone, typically in good yield.

Many methyl ketones are available commercially and are suitable for bromination according to the foregoing methods. In addition to commercially-sourced materials, further methyl ketones may be prepared by the following method.

### Conversion of a Carboxylic Acid to a Methyl Ketone Via a Weinreb Amide

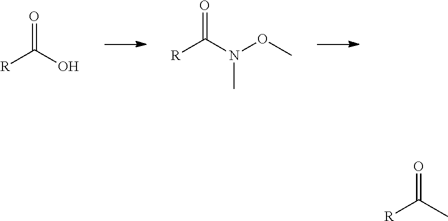

To a solution of substituted carboxylic acid (1.0 eq) in DMF is added N,O-dimethylhydroxylamine hydrochloride (1.10 eq), 1-(3-dimethylaminopropyl)-3-ethylcarbodiimide hydrochloride (1.50 eq), 1-hydroxybenzotriazole (1.0 eq), 4-dimethylaminopyridine (1.0 eq) and a catalytic amount of triethylamine. The resulting reaction mixture is stirred at room temperature. After the completion of reaction (TLC monitoring), ice-cold water is added to the reaction mixture, which is then extracted with EtOAc (3 times). The combined organics are washed with ice-cold water and brine, dried over anhydrous Na₂SO₄, filtered and concentrated in vacuo. If required, the crude product may be purified by silica chromatography (typically eluted with EtOAc/hexane). A solution of the above Weinreb amide (1.0 eq) in THF is cooled to 0° C. and treated with methylmagnesium bromide (2.0 eq). The resulting reaction mixture is stirred at 0° C. for approximately 45 minutes. At the completion of reaction (TLC monitoring), saturated ammonium chloride solution is added to the reaction mixture, followed by extraction with EtOAc (3 times). The combined organics are washed with brine, dried over anhydrous Na₂SO₄, filtered and concentrated in vacuo. The crude residue may be purified by silica chromatography (typically eluted with EtOAc/hexane) to obtain the pure methyl ketone.

### General Method A

One general method for the preparation of compounds of Formula (I) is described in Scheme 1.

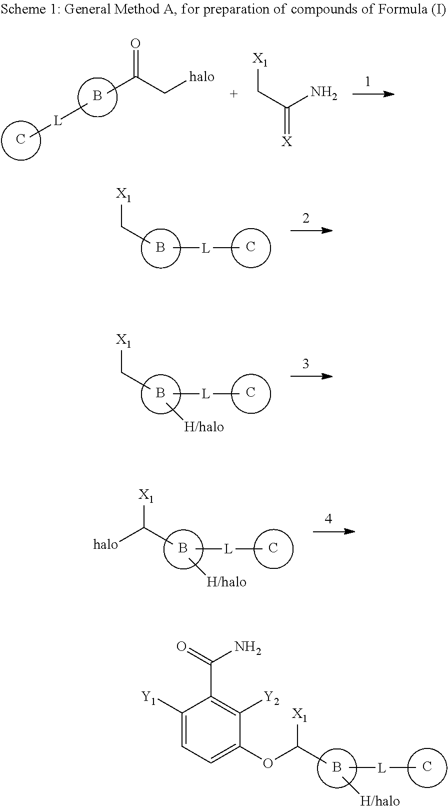

Step 1: Formation of Oxazole/Thiazole Ring; Coupling of Halomethyl Ketones with Amide/Thioamide Derivatives

A mixture of α-halo ketone C-L-B—C(O)—CH-halo (1.0 eq) and amide/thioamide derivative X₁—CH₂—C(X)NH₂ (2.50 eq) is heated at 120-130° C. for 2-3 h. After completion of reaction, water is added to the reaction mass and washed with EtOAc. The combined organics are collected, washed sequentially with water and brine, dried over anhydrous Na₂SO₄, filtered and concentrated in vacuo. The crude residue may be purified by silica chromatography (EtOAc/hexane) to obtain the desired products, typically in moderate to good yields.

### Step 2: Optional Ring Halogenation (Bromo/Chloro)

Approach i):

To a solution of the 5-H oxazolyl or thiazolyl substrate (1.0 eq) in acetic acid (˜5 mL/mmol) is added NBS or NCS (1.0 eq) and the resulting reaction mixture is stirred at room temperature for 30-45 minutes. After the completion of reaction (TLC monitoring), the reaction mass is diluted with water, basified with saturated sodium bicarbonate solution and extracted with EtOAc. The combined organics are washed with brine, dried, filtered and concentrated. The residue may be purified over silica gel to obtain the desired products, typically in moderate yields.

Approach ii):

To an ice-cold solution of a 5-H oxazole derivative (1.0 eq) in DCM (˜10 mL/mmol) is added a stock solution of 1M bromine in DCM (1.0 eq) and the resulting solution is stirred at room temperature for 5-6 h while constantly monitoring the progress of the reaction by TLC/MS. After the majority of the starting material is converted to product, the reaction is quenched by addition of saturated aqueous NaHCO₃ solution followed by extraction with EtOAc (3 times). The combined organics are washed with brine, dried over anhydrous Na₂SO₄, filtered and concentrated under reduced pressure. The crude material may be purified by flash chromatography (EtOAc-hexane) to obtain the desired product, typically in good yield.

### Step 3: Halogenation of the Side Chain on the Heteroaryl Ring

To a solution of the starting material (1.0 eq) in CCl₄ is added NBS (1.0 eq) and AIBN (0.10 eq). The resulting reaction mixture is stirred at 80° C. for 5-6 h. After the completion of reaction (TLC monitoring), the reaction mixture is filtered and concentrated. The crude residue may be purified over silica gel to obtain the pure alkyl halide, typically in moderate yield.

### Step 4: Coupling of the Head Group (2,6-Difluoro-3-Hydroxybenzamide) by Nucleophilic Substitution of an Alkyl Halide

To a solution of the appropriate alkyl halide (1.0 eq) in DMF is added K₂CO₃ (2.0 eq) followed by 2,6-difluoro-3-hydroxybenzamide (1.0 eq). The resulting reaction mixture is stirred under N₂ atmosphere at room temperature for 2 h. After the reaction is complete (TLC monitoring), ice-cold water is added to the reaction mixture and extracted with EtOAc (3 times). The combined organics are washed with 1M NaOH solution, water and brine, dried (Na₂SO₄), filtered and concentrated. The crude residue may be purified by silica chromatography to obtain the desired product, typically in moderate to good yields.

### General Method B

One general method for the preparation of compounds of Formula (I) where X₁ is a hydroxyl or hydroxyalkyl moiety is described in Scheme 2.

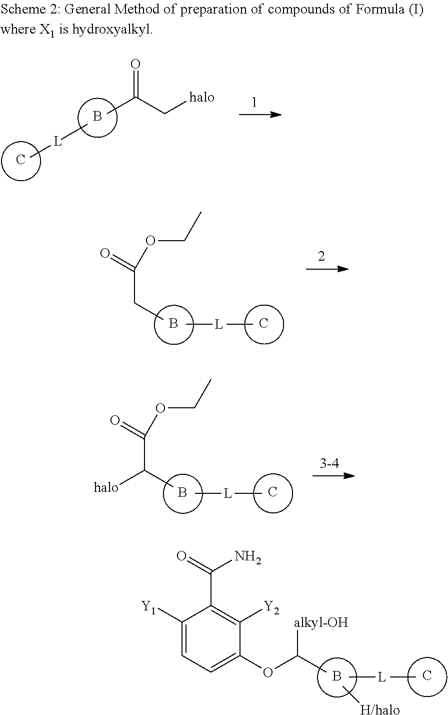

Step 1: Ring formation as per step 1 General Method A.
Step 2: Halogenation as per step 3 General Method A.
Step 3: Coupling as per step 4 General Method A.
Step 4: Reduction of the ester/keto group

To an ice cold solution of the corresponding ester derivative (1.0 eq) in MeOH is added NaBH₄ (3.0 eq) portion wise and the resulting reaction mixture is refluxed for 2 h. After the completion of reaction (TLC monitoring), water is added to the mixture and extracted with EtOAc (3 times). The combined organics are washed with water, brine, dried (Na₂SO₄), filtered and concentrated to obtain the desired product (in good to excellent yields), typically without need for further purification.

In an alternative method the carboxylate produced following step 1 may be converted to a hydroxyl then protected as an acetate during functional group interconversions of substituents on ring B or ring C then deprotected to the hydroxyl following coupling to the benzamide head group.

### General Method C

One general method for the preparation of compounds of Formula (I) wherein X₁ is a carboxylate moiety, e.g. carboxylic acid, esters, acetates, is described in Scheme 3.

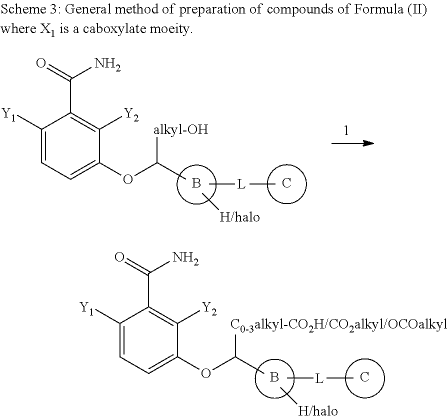

### Step 1: Carboxyl Formation

To a solution of the starting material having the primary hydroxyl group in DCM is added a suitable base such as imidazole (1.20 eq) and an acylating agent, such as an activated ester, an alkyl halide or an anhydride (1.20 eq). The resulting reaction mixture is stirred at room temperature. After the completion of reaction (TLC monitoring), water is added followed by extraction with EtOAc (3 times). The combined organics are washed with brine, dried (Na₂SO₄), filtered and concentrated under reduced pressure. The residue may be purified by chromatography to obtain the desired product.

Alternatively oxidation conditions such as CrO₃, H₂SO₄, acetone (Jones Reagent) may be employed to form carboxylates from starting materials having the primary hydroxyl group. Other suitable acylating or oxidizing reagents and conditions will be familiar to the skilled person.

### General Method D

One general method for the preparation of compounds of Formula (Id) is described in Scheme 4.

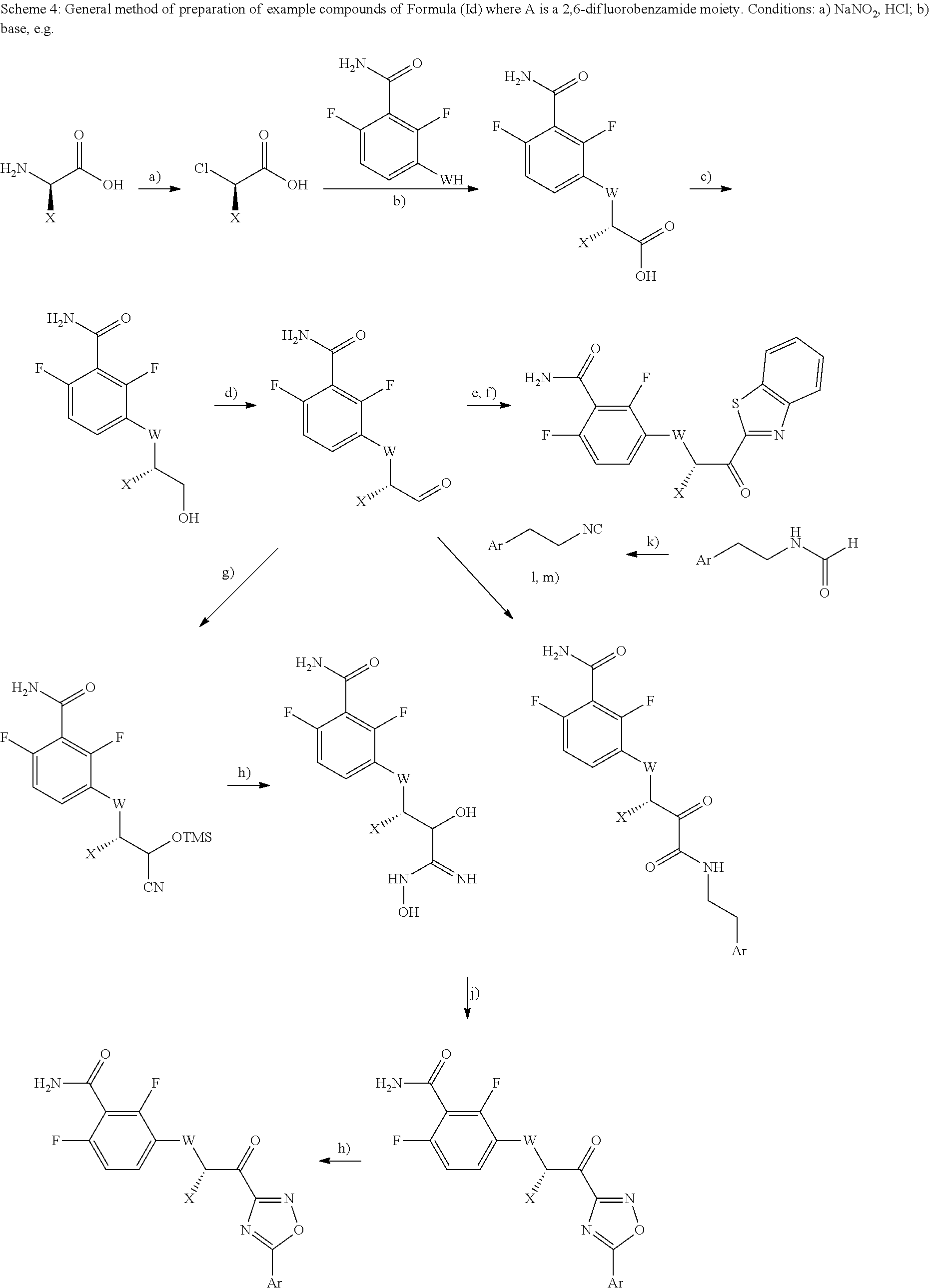

10% NaOH; c) i) NMM, IBCF, THF, −10° C.; ii) NaBH₄, H₂O, 0° C.; d) COCI₂, DMSO, Et₃N, CH₂Cl₂; e) 1,3-benzothiazole, BuLi, THF, −78° C.; f) Dess-Martin periodinane, CH₂Cl₂; g) TMSCN; h) NH₂OH, EtOH; j) ArCO₂H, DCC, then pyridine, 120° C.; k) Burgess reagent; 1) TFA, CH₂Cl₂; m) i) K₂CO₃, MeOH; ii) Dess-Martin periodinane, CH₂Cl₂.

### Functional Group Interconversions

The skilled person will appreciate that a wide diversity of compounds may be provided by functional group interconversions of hydroxyls and carboxylates including but not limited to halogens, ethers, ketones, carboxylic acids, esters, carbonates, amines, amides, ureas, carbamates, sulfates, sulfonamides, phosphates, heterocycles, heteroaryls, optionally substituted alkyl chain extensions and so on. Embodiments will now be described.

### EXAMPLES

Those skilled in the art will appreciate that the invention described herein is susceptible to variations and modifications other than those specifically described. The invention will now be described without limitation by reference to the examples which follow.

¹H NMR spectra were recorded on a Bruker Ultrashield™ 400 spectrometer. Spectra were recorded in CDCl₃, d₆-acetone, CD₃CN, CD₃OD or d₆-DMSO using the residual solvent peak as a reference. Chemical shifts are reported on the δ scale in parts per million (ppm) using the following conventions to assign the multiplicity: s (singlet), d (doublet), t (triplet), q (quartet), m (multiplet) and the prefix br (broad). Mass spectra (ESI) were typically recorded on a Thermo Finnigan LCQ Advantage or LCQ Deca mass spectrometer coupled with a Thermo Finnigan Surveyor HPLC system. The HPLC was performed using Waters Acquity UPLC BEH or Phenomenex C8(2) or C18(2) columns. Water containing 0.1% formic acid (solvent A) and acetonitrile containing 0.1% formic acid (solvent B) were used for separations at acidic pH. Ammonium acetate (5 mM, solvent A) and methanol or acetonitrile (solvent B) were used for separations at neutral pH. Flash chromatography was performed on 40-63 μm or 125-250 μm silica gel or using a Biotage SP4 with GraceResolv™ silica cartridge.

Compounds 1-103 as provided below are previously described in WO2007/107758, WO2009/037485 WO2009/040507 and WO2012/142671 and may be prepared in accordance with the methods described therein.

| Cpd | | |
|---|---|---|
| No | Structure | Name (ACD) |
| 1 | 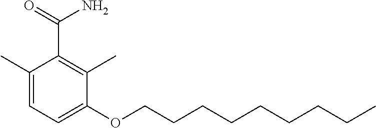 [SMILES: CCCCCCCCCOc1ccc(C)c(C(N)=O)c1C] | 2,6-difluoro-3-(nonyloxy)benzamide |
| 2 | 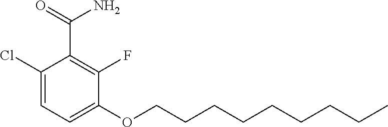 [SMILES: CCCCCCCCCOc1ccc(Cl)c(C(N)=O)c1F] | 6-chloro-2-fluoro-3-(nonyloxy)benzamide |
| 3 | 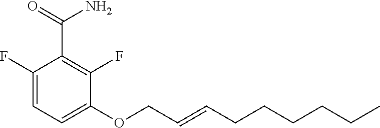 [SMILES: CCCCCC/C=C/COc1ccc(F)c(C(N)=O)c1F] | 2,6-difluoro-3-[(2E)-non-2-en-1-yloxy]benzamide |
| 4 | 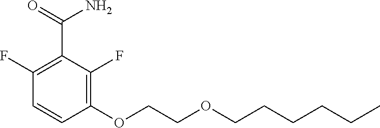 [SMILES: CCCCCCOCCOc1ccc(F)c(C(N)=O)c1F] | 2,6-difluoro-3[2-(hexyloxy)ethoxy]benzamide |
| 5 | 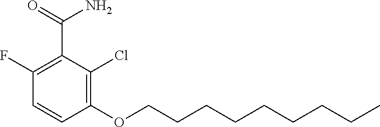 [SMILES: CCCCCCCCCOc1ccc(F)c(C(N)=O)c1Cl] | 2-chloro-6-fluoro-3-(nonyloxy)benzamide |
| 6 | 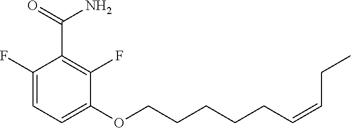 [SMILES: CC/C=C\CCCCCOc1ccc(F)c(C(N)=O)c1F] | 2,6-difluoro-3-[(6Z)-non-6-en-1-yloxy]benzamide |
| 7 | 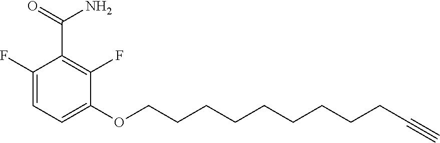 [SMILES: C#CCCCCCCCCCOc1ccc(F)c(C(N)=O)c1F] | 2,6-difluoro-3-(undec-10-yn-1-yloxy)benzamide |
| 8 | 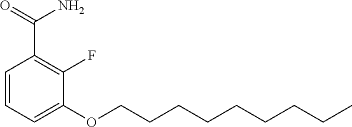 [SMILES: CCCCCCCCCOc1cccc(C(N)=O)c1F] | 2-fluoro-3-(nonyloxy)benzamide |
| 9 | 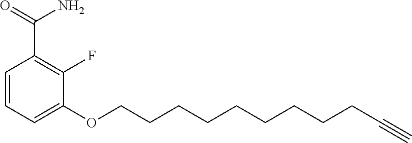 [SMILES: C#CCCCCCCCCCOc1cccc(C(N)=O)c1F] | 2-fluoro-3-(undec-10-yn-1-yloxy)benzamide |
| 10 | 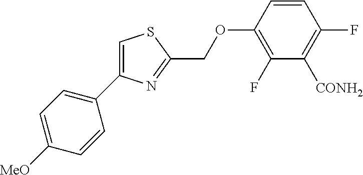 [SMILES: COc1ccc(-c2csc(COc3ccc(F)c(C(N)=O)c3F)n2)cc1] | 2,6-difluoro-3-{[4-(4-methoxyphenyl)-1,3-thiazol-2- yl]methoxy}benzamide |
| 11 | 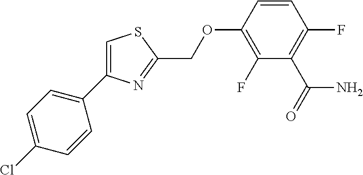 [SMILES: NC(=O)c1c(F)ccc(OCc2nc(-c3ccc(Cl)cc3)cs2)c1F] | 3-{[4-(4-chlorophenyl)-1,3-thiazol-2-yl]methoxy}-2,6- difluorobenzamide |
| 12 | 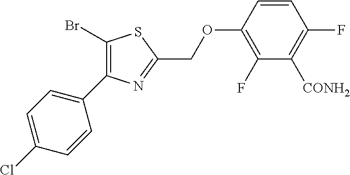 [SMILES: NC(=O)c1c(F)ccc(OCc2nc(-c3ccc(Cl)cc3)c(Br)s2)c1F] | 3-{[5-bromo-4-(4-chlorophenyl)-1,3-thiazol-2- yl]methoxy}-2,6-difluorobenzamide |
| 13 | 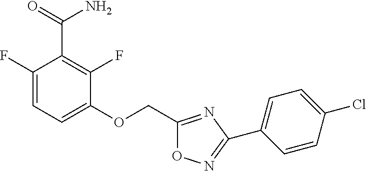 [SMILES: NC(=O)c1c(F)ccc(OCc2nc(-c3ccc(Cl)cc3)no2)c1F] | 3-{[3-(4-chlorophenyl)-1,2,4-oxadiazol-5- yl]methoxy}-2,6-difluorobenzamide |
| 14 | 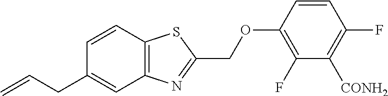 [SMILES: C=CCc1ccc2sc(COc3ccc(F)c(C(N)=O)c3F)nc2c1] | 2,6-difluoro-3-{[5-(prop-2-en-1-yl)-1,3-benzothiazol-2- yl]methoxy}benzamide |
| 15 | 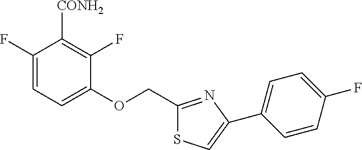 [SMILES: NC(=O)c1c(F)ccc(OCc2nc(-c3ccc(F)cc3)cs2)c1F] | 2,6-difluoro-3-{[4-(4-fluorophenyl)-1,3-thiazol-2- yl]methoxy}benzamide |
| 16 | 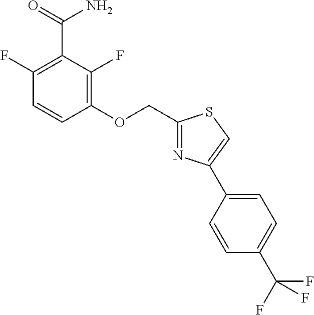 [SMILES: NC(=O)c1c(F)ccc(OCc2nc(-c3ccc(C(F)(F)F)cc3)cs2)c1F] | 2,6-difluoro-3-({4-[4-(trifluoromethyl)phenyl]-1,3- thiazol-2-yl}methoxy)benzamide |
| 17 | 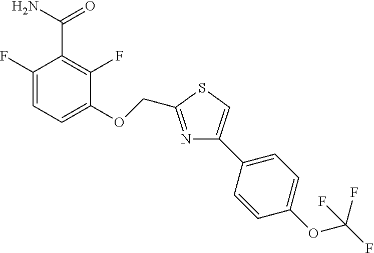 [SMILES: NC(=O)c1c(F)ccc(OCc2nc(-c3ccc(OC(F)(F)F)cc3)cs2)c1F] | 2,6-difluoro-3-({4-[4-(trifluoromethoxy)phenyl]-1,3- thiazol-2-yl}methoxy)benzamide |
| 18 | 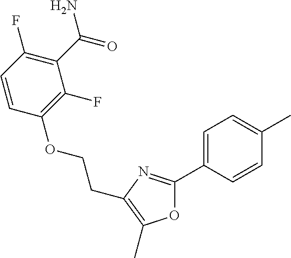 [SMILES: Cc1ccc(-c2nc(CCOc3ccc(F)c(C(N)=O)c3F)c(C)o2)cc1] | 2,6-difluoro-3-{2-[5-methyl-2-(4-methylphenyl)-1,3- oxazol-4-yl]ethoxy}benzamide |
| 19 | 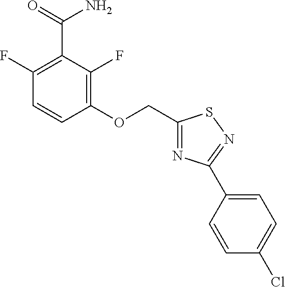 [SMILES: NC(=O)c1c(F)ccc(OCc2nc(-c3ccc(Cl)cc3)ns2)c1F] | 3-{[3-(4-chlorophenyl)-1,2,4-thiadiazol-5- yl]methoxy}-2,6-difluorobenzamide |
| 20 | 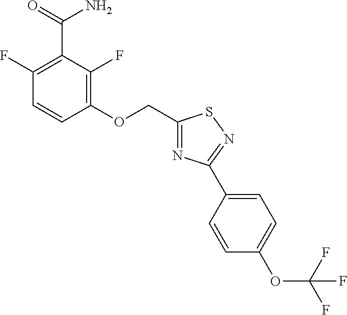 [SMILES: NC(=O)c1c(F)ccc(OCc2nc(-c3ccc(OC(F)(F)F)cc3)ns2)c1F] | 2,6-difluoro-3-({3[4-(trifluoromethoxy)phenyl]-1,2,4- thiadiazol-5-yl}methoxy)benzamide |
| 21 | 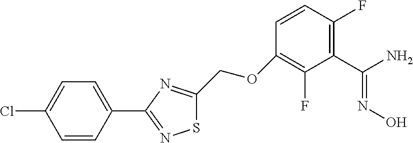 [SMILES: N/C(=N\O)c1c(F)ccc(OCc2nc(-c3ccc(Cl)cc3)ns2)c1F] | 3-{[3-(4-chlorophenyl)-1,2,4-thiadiazol-5- yl]methoxy}-2,6-difluoro-N′-hydroxybenzene- carboximidamide |
| 22 | 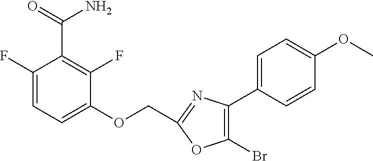 [SMILES: COc1ccc(-c2nc(COc3ccc(F)c(C(N)=O)c3F)oc2Br)cc1] | 3-{[5-bromo-4-(4-methoxyphenyl)-1,3-oxazo1-2- yl]methoxy}-2,6-difluorobenzamide |
| 23 | 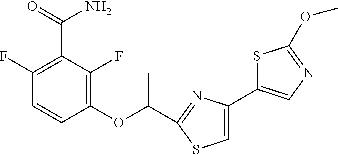 [SMILES: COc1ncc(-c2csc(C(C)Oc3ccc(F)c(C(N)=O)c3F)n2)s1] | 2,6-difluoro-3-[1-(2′-methoxy-4,5′-bi-1,3-thiazol-2- yl)ethoxy]benzamide |
| 24 | 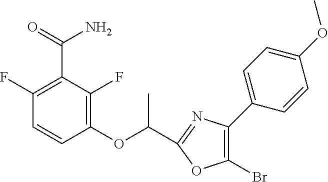 [SMILES: COc1ccc(-c2nc(C(C)Oc3ccc(F)c(C(N)=O)c3F)oc2Br)cc1] | 3-{1-[5-bromo-4-(4-methoxyphenyl)-1,3-oxazol-2- yl]ethoxy}-2,6-difluorobenzamide |
| 25 | 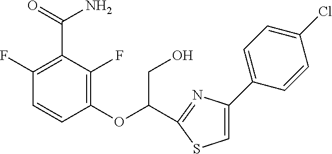 [SMILES: NC(=O)c1c(F)ccc(OC(CO)c2nc(-c3ccc(Cl)cc3)cs2)c1F] | 3-{1-[4-(4-chlorophenyl)-1,3-thiazol-2-yl]-2- hydroxyethoxy}-2,6-difluorobenzamide |
| 26 | 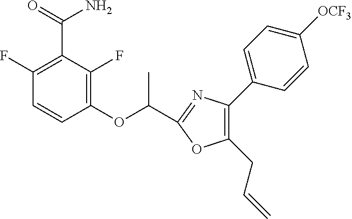 [SMILES: C=CCc1oc(C(C)Oc2ccc(F)c(C(N)=O)c2F)nc1-c1ccc(OC(F)(F)F)cc1] | 2,6-difluoro-3-(1-{5-(prop-2-en-1-yl)-4-[4- (trifluoromethoxy)phenyl]-1,3-oxazol-2- yl}ethoxy)benzamide |
| 27 | 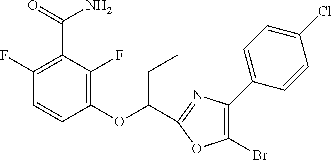 [SMILES: CCC(Oc1ccc(F)c(C(N)=O)c1F)c1nc(-c2ccc(Cl)cc2)c(Br)o1] | 3-{1-[5-bromo-4-(4-chlorophenyl)-1,3-oxazol-2- yl]propoxy}-2,6-difluorobenzamide |
| 28 | 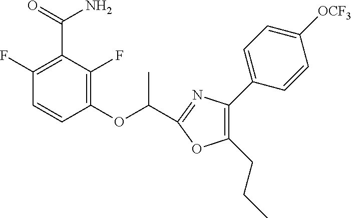 [SMILES: CCCc1oc(C(C)Oc2ccc(F)c(C(N)=O)c2F)nc1-c1ccc(OC(F)(F)F)cc1] | 2,6-difluoro-3-(1-{5-propyl-4-[4- (trifluoromethoxy)phenyl]-1,3-oxazol-2- yl}ethoxy)benzamide |
| 29 | 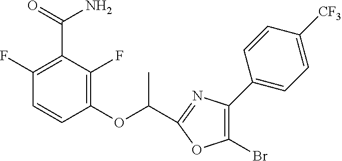 [SMILES: CC(Oc1ccc(F)c(C(N)=O)c1F)c1nc(-c2ccc(C(F)(F)F)cc2)c(Br)o1] | 3-(1-{5-bromo-4-[4-(trifluoromethyl)phenyl]-1,3- oxazol-2-yl}ethoxy)-2,6-difluorobenzamide |
| 30 | 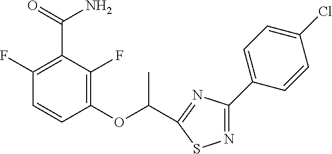 [SMILES: CC(Oc1ccc(F)c(C(N)=O)c1F)c1nc(-c2ccc(Cl)cc2)ns1] | 3-{1-[3-(4-chlorophenyl)-1,2,4-thiadiazol-5- yl]ethoxy}-2,6-difluorobenzamide |
| 31 | 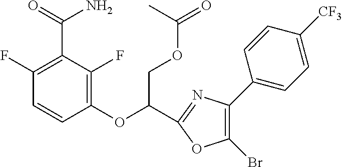 [SMILES: CC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl acetate |
| 32 | 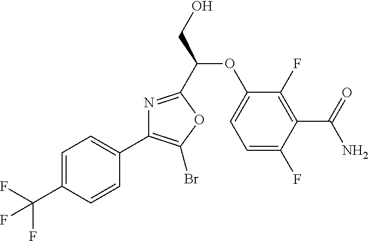 [SMILES: NC(=O)c1c(F)ccc(O[C@H](CO)c2nc(-c3ccc(C(F)(F)F)cc3)c(Br)o2)c1F] | 3-(1-{5-bromo-4-[4-(trifluoromethyl)phenyl]-1,3- oxazol-2-yl}-2-hydroxyethoxy)-2,6-difluorobenzamide |
| 33 | 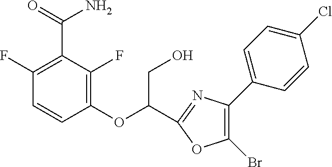 [SMILES: NC(=O)c1c(F)ccc(OC(CO)c2nc(-c3ccc(Cl)cc3)c(Br)o2)c1F] | 3-{1-[5-bromo-4-(4-chlorophenyl)-1,3-oxazol-2-yl]-2- hydroxyethoxy}-2,6-difluorobenzamide |
| 34 | 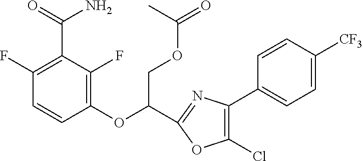 [SMILES: CC(=O)OCC(Oc1ccc(F)c(C(N)=O)c1F)c1nc(-c2ccc(C(F)(F)F)cc2)c(Cl)o1] | 2-(3-carbamoyl-2,4-difluorophenoxy)-2-{5-chloro-4- [4-(trifluoromethyl)phenyl]-1,3-oxazol-2-yl}ethyl acetate |
| 35 | 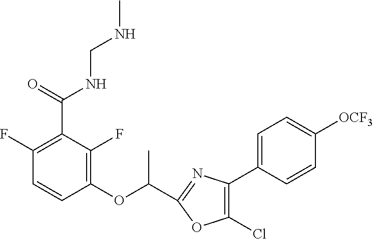 [SMILES: CNCNC(=O)c1c(F)ccc(OC(C)c2nc(-c3ccc(OC(F)(F)F)cc3)c(Cl)o2)c1F] | 3-(1-{5-chloro-4-[4-(trifluoromethoxy)phenyl]-1,3- oxazol-2-yl}ethoxy)-2,6-difluoro-N- [(methylamino)methyl]benzamide |
| 36 | 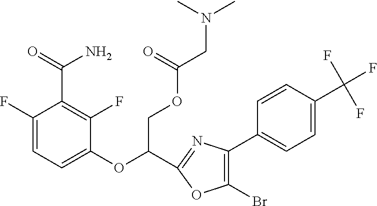 [SMILES: CN(C)CC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl N,N- dimethylglycinate |
| 37 | 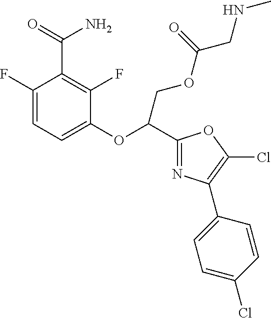 [SMILES: CNCC(=O)OCC(Oc1ccc(F)c(C(N)=O)c1F)c1nc(-c2ccc(Cl)cc2)c(Cl)o1] | 2-(3-carbamoyl-2,4-difluorophenoxy)-2-[5-chloro-4-(4- chlorophenyl)-1,3-oxazol-2-yl]ethyl N-methylglycinate |
| 38 | 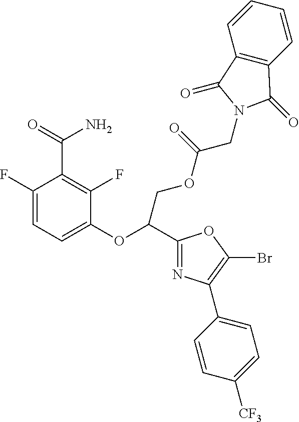 [SMILES: NC(=O)c1c(F)ccc(OC(COC(=O)CN2C(=O)c3ccccc3C2=O)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl (1,3- dioxo-1,3-dihydro-2H-isoindo1-2-yl)acetate |
| 39 | 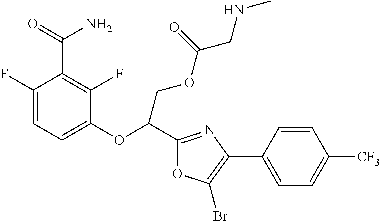 [SMILES: CNCC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl N- methylglycinate |
| 40 | 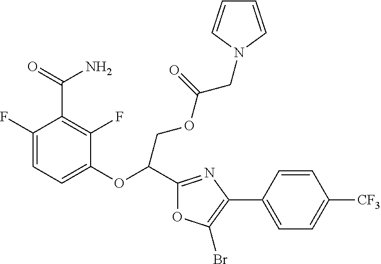 [SMILES: NC(=O)c1c(F)ccc(OC(COC(=O)Cn2cccc2)c2nc(-c3ccc(C(F)(F)F)cc3)c(Br)o2)c1F] | 2-{5-bromo-4-[4-(trifluoromethyl)phenyl]-1,3- oxazol-2-yl}-2-(3-carbamoyl-2,4-difluorophenoxy)ethyl 1H- pyrrol-l-ylacetate |
| 41 | 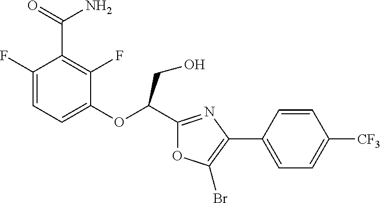 [SMILES: NC(=O)c1c(F)ccc(O[C@@H](CO)c2nc(-c3ccc(C(F)(F)F)cc3)c(Br)o2)c1F] | 3-[(1S)-1-{5-bromo-4-[4-(trifluoromethyl)phenyl]-1,3- oxazol-2-yl}-2-hydroxyethoxy]-2,6-difluorobenzamide |
| 42 | 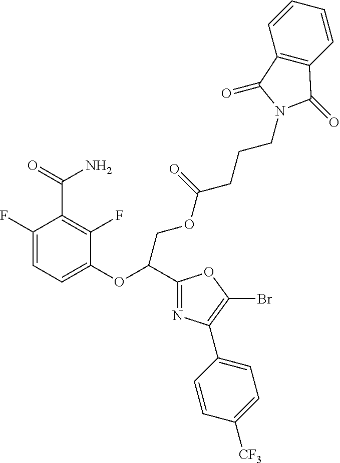 [SMILES: NC(=O)c1c(F)ccc(OC(COC(=O)CCCN2C(=O)c3ccccc3C2=O)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl 4- (1,3-dioxo-1,3-dihydro-2H-isoindo1-2-yl)butanoate |
| 43 | 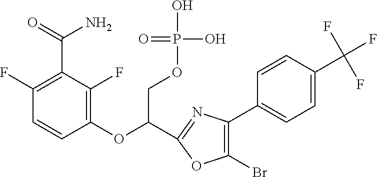 [SMILES: NC(=O)c1c(F)ccc(OC(COP(=O)(O)O)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl dihydrogen phosphate |
| 44 | 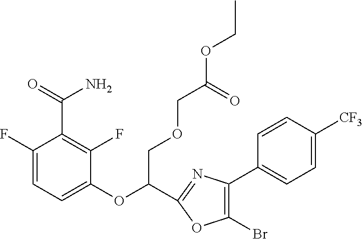 [SMILES: CCOC(=O)COCC(Oc1ccc(F)c(C(N)=O)c1F)c1nc(-c2ccc(C(F)(F)F)cc2)c(Br)o1] | ethyl [2-{5-bromo-4-[4-(trifluoromethyl)phenyl]-1,3- oxazol-2-yl}-2-(3-carbamoyl-2,4- difluorophenoxy)ethoxy]acetate |
| 45 | 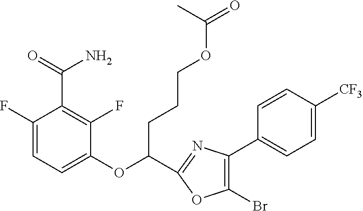 [SMILES: CC(=O)OCCCC(Oc1ccc(F)c(C(N)=O)c1F)c1nc(-c2ccc(C(F)(F)F)cc2)c(Br)o1] | 4-{5-bromo-4-[4-(trifluoromethyl)phenyl]-1,3-oxazol- 2-yl}-4-(3-carbamoyl-2,4-difluorophenoxy)butyl acetate |
| 46 | 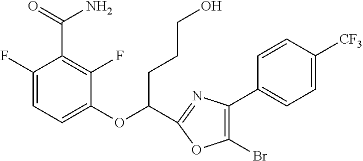 [SMILES: NC(=O)c1c(F)ccc(OC(CCCO)c2nc(-c3ccc(C(F)(F)F)cc3)c(Br)o2)c1F] | 3-(1-{5-bromo-4-[4-(trifluoromethyl)phenyl]-1,3- oxazol-2-yl}-4-hydroxybutoxy)-2,6-difluorobenzamide |
| 47 | 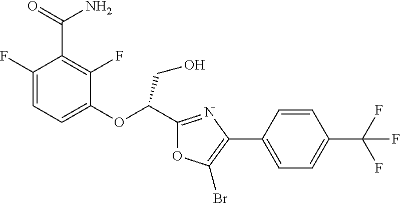 [SMILES: NC(=O)c1c(F)ccc(O[C@H](CO)c2nc(-c3ccc(C(F)(F)F)cc3)c(Br)o2)c1F] | 3-[(1R)-1-{5-bromo-4-[4-(trifluoromethyl)phenyl]-1,3- oxazol-2-yl}-2-hydroxyethoxy]-2,6-difluorobenzamide |
| 48 | 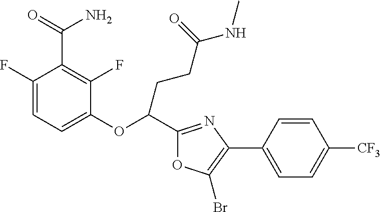 [SMILES: CNC(=O)CCC(Oc1ccc(F)c(C(N)=O)c1F)c1nc(-c2ccc(C(F)(F)F)cc2)c(Br)o1] | 3-[1-{5-bromo-4-[4-(trifluoromethyl)phenyl]-1,3- oxazol-2-yl}-4-(methylamino)-4-oxobutoxy]-2,6- difluorobenzamide |
| 49 | 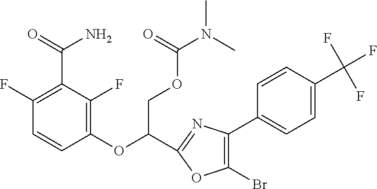 [SMILES: CN(C)C(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl dimethylcarbamate |
| 50 | 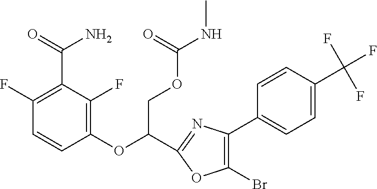 [SMILES: CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl methylcarbamate |
| 51 | 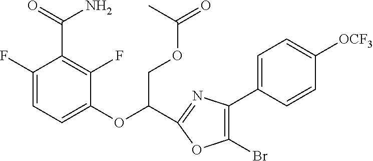 [SMILES: CC(=O)OCC(Oc1ccc(F)c(C(N)=O)c1F)c1nc(-c2ccc(OC(F)(F)F)cc2)c(Br)o1] | 2-{5-bromo-4-[4-(trifluoromethoxy)phenyl]-1,3- oxazol-2-yl}-2-(3-carbamoyl-2,4- difluorophenoxy)ethyl acetate |
| 52 | 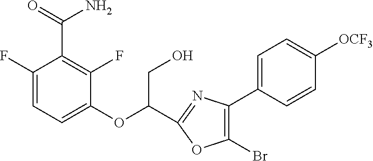 [SMILES: NC(=O)c1c(F)ccc(OC(CO)c2nc(-c3ccc(OC(F)(F)F)cc3)c(Br)o2)c1F] | 3-(1-{5-bromo-4-[4-(trifluoromethoxy)phenyl]-1,3- oxazol-2-yl}-2-hydroxyethoxy)-2,6-difluorobenzamide |
| 53 | 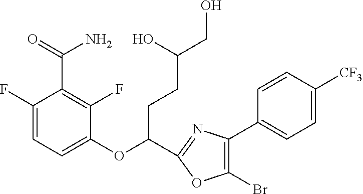 [SMILES: NC(=O)c1c(F)ccc(OC(CCC(O)CO)c2nc(-c3ccc(C(F)(F)F)cc3)c(Br)o2)c1F] | 3-[(1-{5-bromo-4-[4-(trifluoromethyl)phenyl]-1,3- oxazol-2-yl}-4,5-dihydroxypentyl)oxy]-2,6- difluorobenzamide |
| 54 | 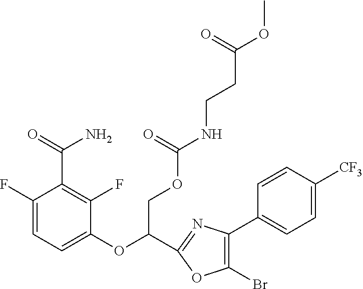 [SMILES: COC(=O)CCNC(=O)OCC(Oc1ccc(F)c(C(N)=O)c1F)c1nc(-c2ccc(C(F)(F)F)cc2)c(Br)o1] | methyl N-{[2-{5-bromo-4-[4-(trifluoromethyl)phenyl]- 1,3-oxazol-2-yl}-2-(3-carbamoyl-2,4- difluorophenoxy)ethoxy]carbonyl}-beta-alaninate |
| 55 | 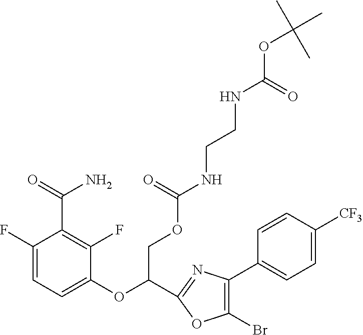 [SMILES: CC(C)(C)OC(=O)NCCNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl tert- butyl ethane-1,2-diylbiscarbamate |
| 56 | 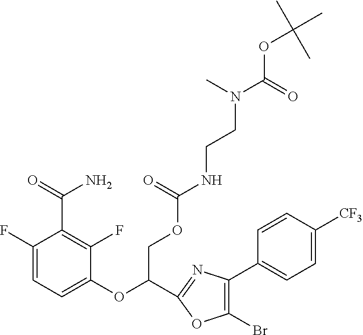 [SMILES: CN(CCNC(=O)OCC(Oc1ccc(F)c(C(N)=O)c1F)c1nc(-c2ccc(C(F)(F)F)cc2)c(Br)o1)C(=O)OC(C)(C)C] | tert-butyl [2-({[2-{5-bromo-4-[4- (trifluoromethyl)phenyl]-1,3-oxazol-2-yl}-2-(3- carbamoyl-2,4- difluorophenoxy)ethoxy]carbonyl}amino)ethyl]methyl carbamate (non-preferred name) |
| 57 | 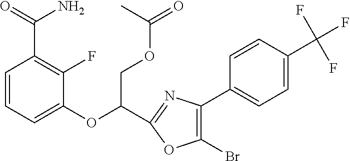 [SMILES: CC(=O)OCC(Oc1cccc(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2-fluorophenoxy)ethyl acetate |
| 58 | 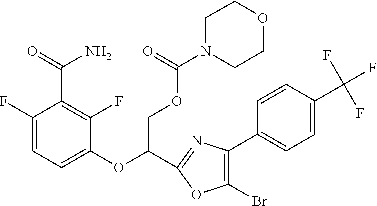 [SMILES: NC(=O)c1c(F)ccc(OC(COC(=O)N2CCOCC2)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl morpholine-4-carboxylate |
| 59 | 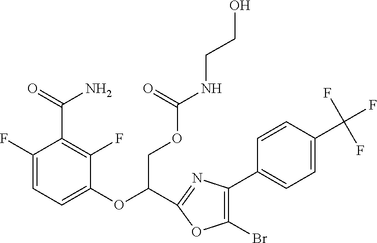 [SMILES: NC(=O)c1c(F)ccc(OC(COC(=O)NCCO)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl (2- hydroxyethyl)carbamate |
| 60 | 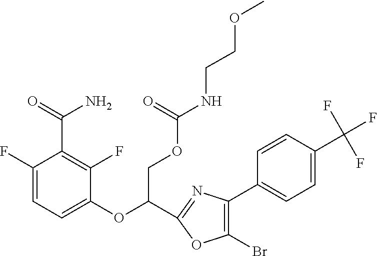 [SMILES: COCCNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl (2- methoxyethyl)carbamate |
| 61 | 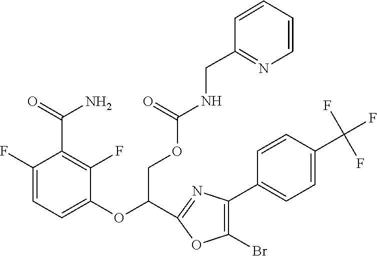 [SMILES: NC(=O)c1c(F)ccc(OC(COC(=O)NCc2ccccn2)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl (pyridin-2-ylmethyl)carbamate |
| 62 | 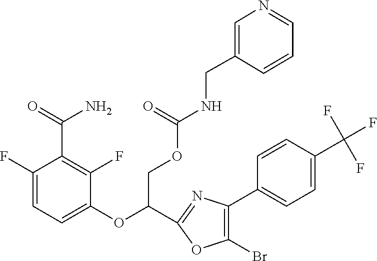 [SMILES: NC(=O)c1c(F)ccc(OC(COC(=O)NCc2cccnc2)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl (pyridin-3-ylmethyl)carbamate |
| 63 | 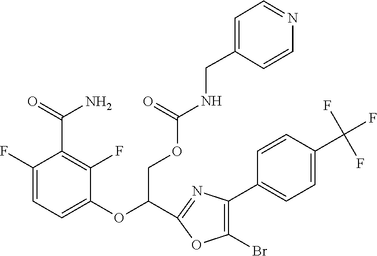 [SMILES: NC(=O)c1c(F)ccc(OC(COC(=O)NCc2ccncc2)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl (pyridin-4-ylmethyl)carbamate |
| 64 | 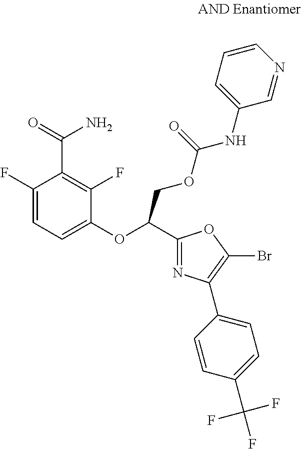 [SMILES: NC(=O)c1c(F)ccc(O[C@@H](COC(=O)Nc2cccnc2)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl pyridin-3-ylcarbamate |
| 65 | 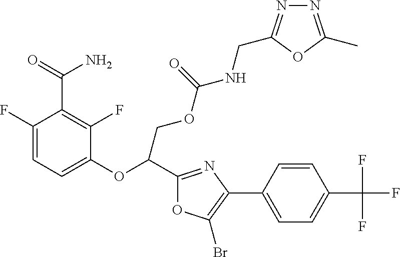 [SMILES: Cc1nnc(CNC(=O)OCC(Oc2ccc(F)c(C(N)=O)c2F)c2nc(-c3ccc(C(F)(F)F)cc3)c(Br)o2)o1] | 2-{5-bromo-4-[4-(trifluoromethyl)phenyl]-1,3-oxazol- 2-yl}-2-(3-carbamoyl-2,4-difluorophenoxy)ethyl [(5- methyl-1,3,4-oxadiazol-2-yl)methyl]carbamate |
| 66 | 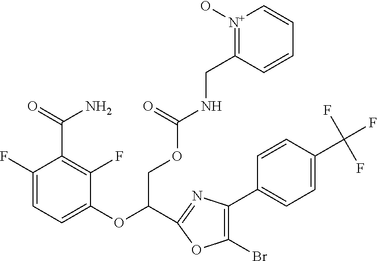 [SMILES: NC(=O)c1c(F)ccc(OC(COC(=O)NCc2cccc[n+]2O)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl [(1- oxidopyridin-2-yl)methyl]carbamate |
| 67 | 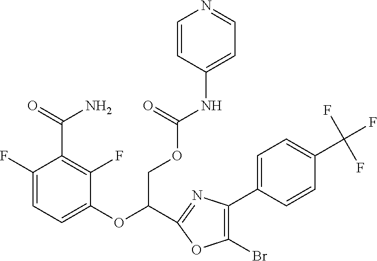 [SMILES: NC(=O)c1c(F)ccc(OC(COC(=O)Nc2ccncc2)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl pyridin-4-ylcarbamate |
| 68 | 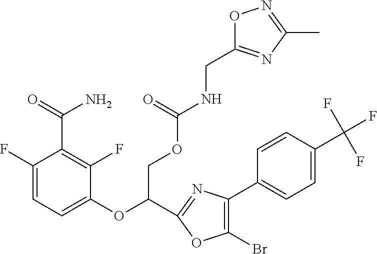 [SMILES: Cc1noc(CNC(=O)OCC(Oc2ccc(F)c(C(N)=O)c2F)c2nc(-c3ccc(C(F)(F)F)cc3)c(Br)o2)n1] | 2-{5-bromo-4-[4-(trifluoromethyl)phenyl]-1,3-oxazol- 2-yl}-2-(3-carbamoyl-2,4-difluorophenoxy)ethyl [(3- methyl-1,2,4-oxadiazol-5-yl)methyl]carbamate |
| 69 | 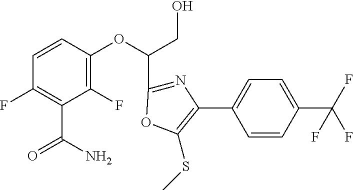 [SMILES: CSc1oc(C(CO)Oc2ccc(F)c(C(N)=O)c2F)nc1-c1ccc(C(F)(F)F)cc1] | 2,6-difluoro-3-(2-hydroxy-1-{5-(methylthio)-4-[4- (trifluoromethyl)phenyl]-1,3-oxazol-2- yl}ethoxy)benzamide |
| 70 | 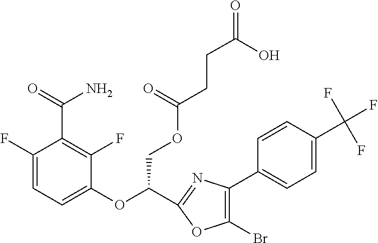 [SMILES: NC(=O)c1c(F)ccc(O[C@H](COC(=O)CCC(=O)O)c2nc(-c3ccc(C(F)(F)F)cc3)c(Br)o2)c1F] | 4-[(2R)-2-{5-bromo-4-[4-(trifluoromethyl)phenyl]-1,3- oxazol-2-yl}-2-(3-carbamoyl-2,4- difluorophenoxy)ethoxy]-4-oxobutanoic acid |
| 71 | 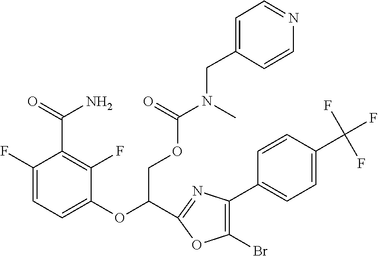 [SMILES: CN(Cc1ccncc1)C(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl methyl(pyridin-4-ylmethyl)carbamate |
| 72 | 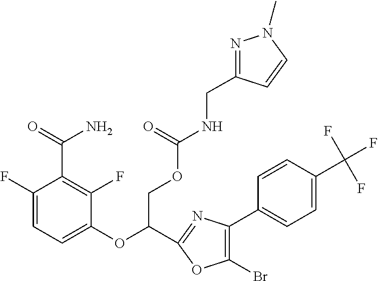 [SMILES: Cn1ccc(CNC(=O)OCC(Oc2ccc(F)c(C(N)=O)c2F)c2nc(-c3ccc(C(F)(F)F)cc3)c(Br)o2)n1] | 2-{5-bromo-4-[4-(trifluoromethyl)phenyl]-1,3-oxazol- 2-yl}-2-(3-carbamoyl-2,4-difluorophenoxy)ethyl [(1- methyl-1H-pyrazol-3-yl)methyl]carbamate |
| 73 | 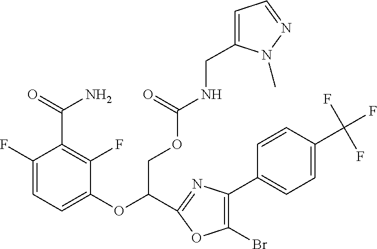 [SMILES: Cn1nccc1CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl [(1- methyl-1H-pyrazo1-5-yl)methyl]carbamate |
| 74 | 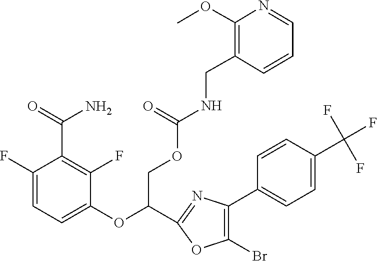 [SMILES: COc1ncccc1CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl [(2- methoxypyridin-3-yl)methyl]carbamate |
| 75 | 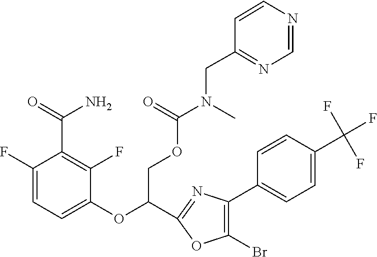 [SMILES: CN(Cc1ccncn1)C(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl methyl(pyrimidin-4-ylmethyl)carbamate |
| 76 | 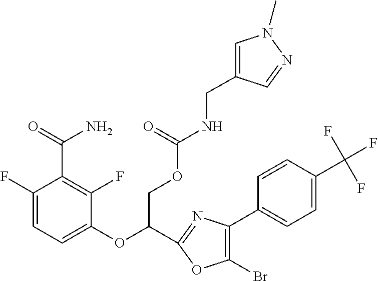 [SMILES: Cn1cc(CNC(=O)OCC(Oc2ccc(F)c(C(N)=O)c2F)c2nc(-c3ccc(C(F)(F)F)cc3)c(Br)o2)cn1] | 2-{5-bromo-4-[4-(trifluoromethyl)phenyl]-1,3-oxazol- 2-yl}-2-(3-carbamoyl-2,4-difluorophenoxy)ethyl [(1- methyl-1H-pyrazol-4-yl)methyl]carbamate |
| 77 | 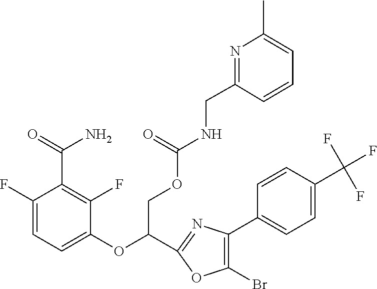 [SMILES: Cc1cccc(CNC(=O)OCC(Oc2ccc(F)c(C(N)=O)c2F)c2nc(-c3ccc(C(F)(F)F)cc3)c(Br)o2)n1] | 2-{5-bromo-4-[4-(trifluoromethyl)phenyl]-1,3-oxazol- 2-yl}-2-(3-carbamoyl-2,4-difluorophenoxy)ethyl [(6- methylpyridin-2-yl)methyl]carbamate |
| 78 | 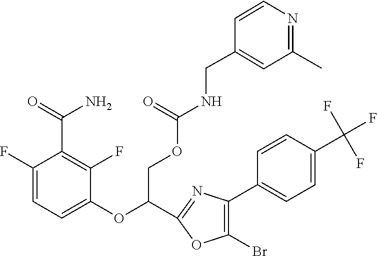 [SMILES: Cc1cc(CNC(=O)OCC(Oc2ccc(F)c(C(N)=O)c2F)c2nc(-c3ccc(C(F)(F)F)cc3)c(Br)o2)ccn1] | 2-{5-bromo-4-[4-(trifluoromethyl)phenyl]-1,3-oxazol- 2-yl}-2-(3-carbamoyl-2,4-difluorophenoxy)ethyl [(2- methylpyridin-4-yl)methyl]carbamate |
| 79 | 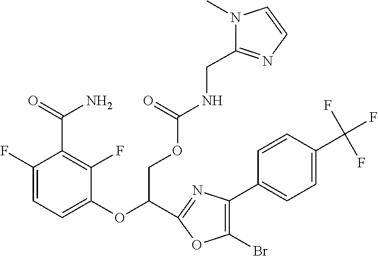 [SMILES: Cn1ccnc1CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl [(1- methyl-1H-imidazol-2-yl)methyl]carbamate |
| 80 | 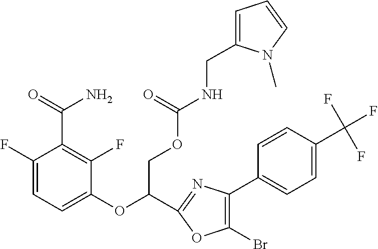 [SMILES: Cn1cccc1CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl [(1- methyl-1H-pyrrol-2-yl)methyl]carbamate |
| 81 | 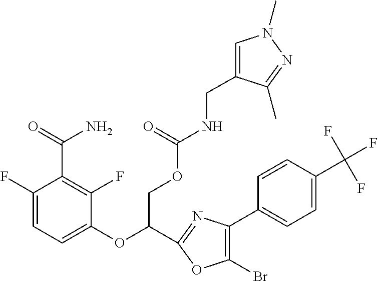 [SMILES: Cc1nn(C)cc1CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl [(1,3- dimethyl-1H-pyrazol-4-yl)methyl]carbamate |
| 82 | 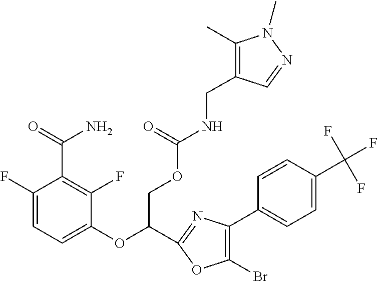 [SMILES: Cc1c(CNC(=O)OCC(Oc2ccc(F)c(C(N)=O)c2F)c2nc(-c3ccc(C(F)(F)F)cc3)c(Br)o2)cnn1C] | 2-{5-bromo-4-[4-(trifluoromethyl)phenyl]-1,3-oxazol- 2-yl}-2-(3-carbamoyl-2,4-difluorophenoxy)ethyl [(1,5- dimethyl-1H-pyrazol-4-yl)methyl]carbamate |
| 83 | 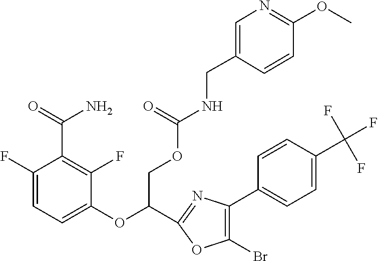 [SMILES: COc1ccc(CNC(=O)OCC(Oc2ccc(F)c(C(N)=O)c2F)c2nc(-c3ccc(C(F)(F)F)cc3)c(Br)o2)cn1] | 2-{5-bromo-4-[4-(trifluoromethyl)phenyl]-1,3-oxazol- 2-yl}-2-(3-carbamoyl-2,4-difluorophenoxy)ethyl [(6- methoxypyridin-3-yl)methyl]carbamate |
| 84 | 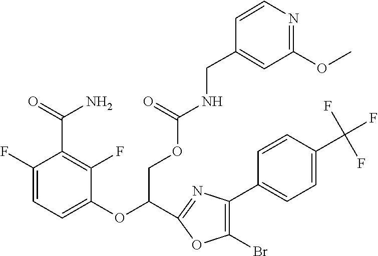 [SMILES: COc1cc(CNC(=O)OCC(Oc2ccc(F)c(C(N)=O)c2F)c2nc(-c3ccc(C(F)(F)F)cc3)c(Br)o2)ccn1] | 2-{5-bromo-4-[4-(trifluoromethyl)phenyl]-1,3-oxazol- 2-yl}-2-(3-carbamoyl-2,4-difluorophenoxy)ethyl [(2- methoxypyridin-4-yl)methyl]carbamate |
| 85 | 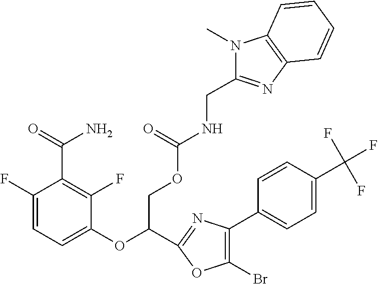 [SMILES: Cn1c(CNC(=O)OCC(Oc2ccc(F)c(C(N)=O)c2F)c2nc(-c3ccc(C(F)(F)F)cc3)c(Br)o2)nc2ccccc21] | 2-{5-bromo-4-[4-(trifluoromethyl)phenyl]-1,3-oxazol-2- yl}-2-(3-carbamoyl-2,4-difluorophenoxy)ethyl [(1- methyl-1H-benzimidazol-2-yl)methyl]carbamate |
| 86 | 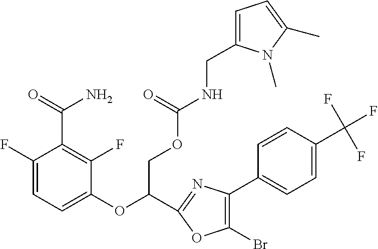 [SMILES: Cc1ccc(CNC(=O)OCC(Oc2ccc(F)c(C(N)=O)c2F)c2nc(-c3ccc(C(F)(F)F)cc3)c(Br)o2)n1C] | 2-{5-bromo-4-[4-(trifluoromethyl)phenyl]-1,3-oxazol- 2-yl}-2-(3-carbamoyl-2,4-difluorophenoxy)ethyl [(1,5- dimethyl-1H-pyrrol-2-yl)methyl]carbamate |
| 87 | 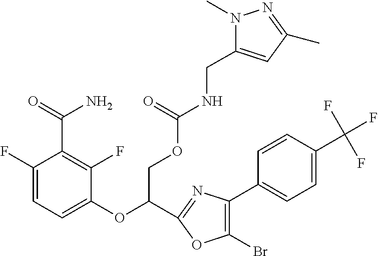 [SMILES: Cc1cc(CNC(=O)OCC(Oc2ccc(F)c(C(N)=O)c2F)c2nc(-c3ccc(C(F)(F)F)cc3)c(Br)o2)n(C)n1] | 2-{5-bromo-4-[4-(trifluoromethyl)phenyl]-1,3-oxazol- 2-yl}-2-(3-carbamoyl-2,4-difluorophenoxy)ethyl [(1,3- dimethyl-1H-pyrazol-5-yl)methyl]carbamate |
| 88 | 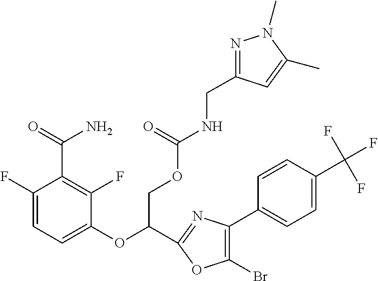 [SMILES: Cc1cc(CNC(=O)OCC(Oc2ccc(F)c(C(N)=O)c2F)c2nc(-c3ccc(C(F)(F)F)cc3)c(Br)o2)nn1C] | 2-{5-bromo-4-[4-(trifluoromethyl)phenyl]-1,3-oxazol- 2-yl}-2-(3-carbamoyl-2,4-difluorophenoxy)ethyl [(1,5- dimethyl-1H-pyrazol-3-yl)methyl]carbamate |
| 89 | 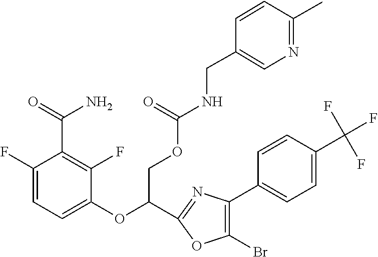 [SMILES: Cc1ccc(CNC(=O)OCC(Oc2ccc(F)c(C(N)=O)c2F)c2nc(-c3ccc(C(F)(F)F)cc3)c(Br)o2)cn1] | 2-{5-bromo-4-[4-(trifluoromethyl)phenyl]-1,3-oxazol- 2-yl}-2-(3-carbamoyl-2,4-difluorophenoxy)ethyl [(6- methylpyridin-3-yl)methyl]carbamate |
| 90 | 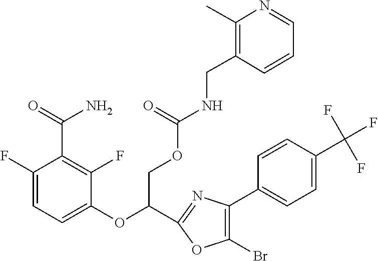 [SMILES: Cc1ncccc1CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]-1,3-oxazol- 2-yl}-2-(3-carbamoyl-2,4-difluorophenoxy)ethyl [(2- methylpyridin-3-yl)methyl]carbamate |
| 91 | 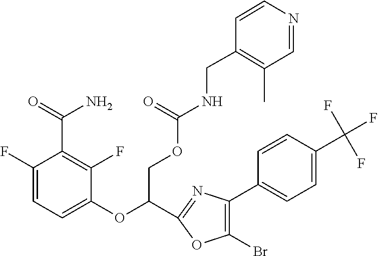 [SMILES: Cc1cnccc1CNC(=O)OCC(Oc1ccc(F)c(C(N)=O)c1F)c1nc(-c2ccc(C(F)(F)F)cc2)c(Br)o1] | 2-{5-bromo-4-[4-(trifluoromethyl)phenyl]1,3-oxazol- 2-yl}-2-(3-carbamoyl-2,4-difluorophenoxy)ethyl [(3- methylpyridin-4-yl)methyl]carbamate |
| 92 | 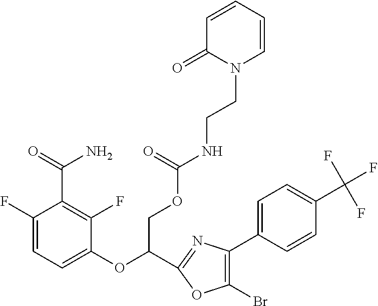 [SMILES: NC(=O)c1c(F)ccc(OC(COC(=O)NCCn2ccccc2=O)c2nc(-c3ccc(C(F)(F)F)cc3)c(Br)o2)c1F] | 2-{5-bromo-4-[4-(trifluoromethyl)phenyl]-1,3-oxazol- 2-yl}-2-(3-carbamoyl-2,4-difluorophenoxy)ethyl [2-(2- oxopyridin-1(2H)-yl)ethyl]carbamate |
| 93 | 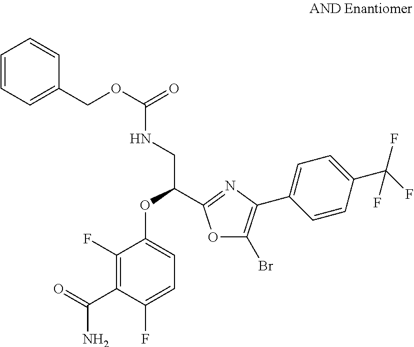 [SMILES: NC(=O)c1c(F)ccc(O[C@@H](CNC(=O)OCc2ccccc2)c2nc(-c3ccc(C(F)(F)F)cc3)c(Br)o2)c1F] | benzyl [2-{5-bromo-4-[4-(trifluoromethyl)phenyl]-1,3- oxazol-2-yl}-2-(3-carbamoyl-2,4- difluorophenoxy)ethyl]carbamate |
| 94 | 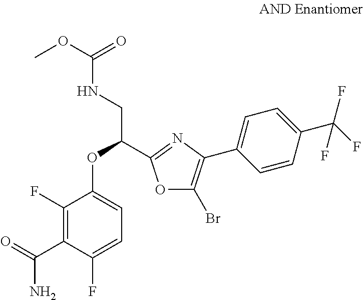 [SMILES: COC(=O)NC[C@H](Oc1ccc(F)c(C(N)=O)c1F)c1nc(-c2ccc(C(F)(F)F)cc2)c(Br)o1] | methyl [2-{5-bromo-4-[4-(trifluoromethyl)phenyl]-1,3- oxazol-2-yl}-2-(3-carbamoyl-2,4- difluorophenoxy)ethyl]carbamate |
| 95 | 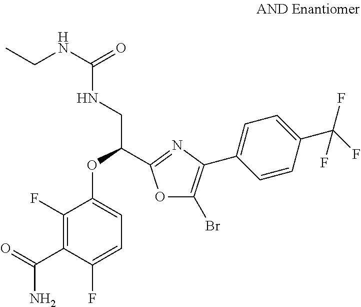 [SMILES: CCNC(=O)NC[C@H](Oc1ccc(F)c(C(N)=O)c1F)c1nc(-c2ccc(C(F)(F)F)cc2)c(Br)o1] | 3-(1-{5-bromo-4-[4-(trifluoromethyl)phenyl]-1,3- oxazol-2-yl}-2-[(ethylcarbamoyl)amino]ethoxy)-2,6- difluorobenzamide |
| 96 | 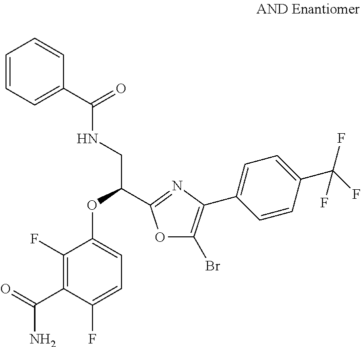 [SMILES: NC(=O)c1c(F)ccc(O[C@@H](CNC(=O)c2ccccc2)c2nc(-c3ccc(C(F)(F)F)cc3)c(Br)o2)c1F] | 3-[2-(benzoylamino)-1-{5-bromo-4-[4- (trifluoromethyl)phenyl]-1,3-oxazol-2-yl}ethoxy]-2,6- difluorobenzamide |
| 97 | 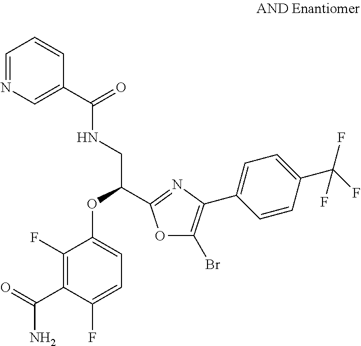 [SMILES: NC(=O)c1c(F)ccc(O[C@@H](CNC(=O)c2cccnc2)c2nc(-c3ccc(C(F)(F)F)cc3)c(Br)o2)c1F] | N-[2-{5-bromo-4-[4-(trifluoromethyl)phenyl]-1,3- oxazol-2-yl}-2-(3-carbamoyl-2,4- difluorophenoxy)ethyl]pyridine-3-carboxamide |
| 98 | 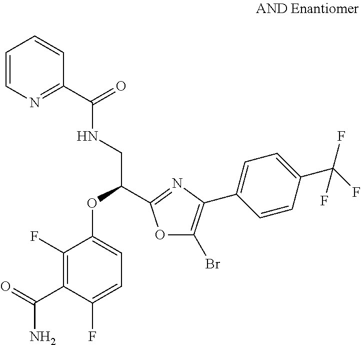 [SMILES: NC(=O)c1c(F)ccc(O[C@@H](CNC(=O)c2ccccn2)c2nc(-c3ccc(C(F)(F)F)cc3)c(Br)o2)c1F] | N-[2-{5-bromo-4-[4-(trifluoromethyl)phenyl]-1,3- oxazol-2-yl}-2-(3-carbamoyl-2,4- difluorophenoxy)ethyl]pyridine-2-carboxamide |
| 99 | 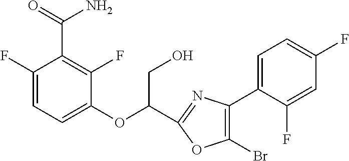 [SMILES: NC(=O)c1c(F)ccc(OC(CO)c2nc(-c3ccc(F)cc3F)c(Br)o2)c1F] | 3-{1-[5-bromo-4-(2,4-difluorophenyl)-1,3-oxazol-2- yl]-2-hydroxyethoxy}-2,6-difluorobenzamide |
| 100 | 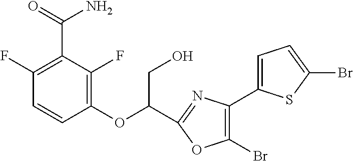 [SMILES: NC(=O)c1c(F)ccc(OC(CO)c2nc(-c3ccc(Br)s3)c(Br)o2)c1F] | 3-{1-[5-bromo-4-(5-bromothiophen-2-yl)-1,3-oxazol- 2-yl]-2-hydroxyethoxy}-2,6-difluorobenzamide |
| 101 | 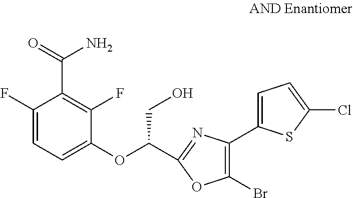 [SMILES: NC(=O)c1c(F)ccc(O[C@H](CO)c2nc(-c3ccc(Cl)s3)c(Br)o2)c1F] | 3-{1-[5-bromo-4-(5-chlorothiophen-2-yl)-1,3-oxazol-2- yl]-2-hydroxyethoxy}-2,6-difluorobenzamide |
| 102 | 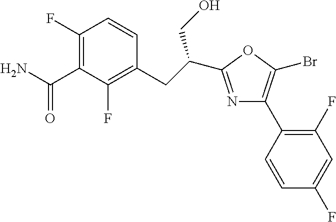 [SMILES: NC(=O)c1c(F)ccc(C[C@H](CO)c2nc(-c3ccc(F)cc3F)c(Br)o2)c1F] | 3-{(1R)-1-[5-bromo-4-(2,4-difluorophenyl)-1,3-oxazol- 2-yl]-2-hydroxyethoxy}-2,6-difluorobenzamide |
| 103 | 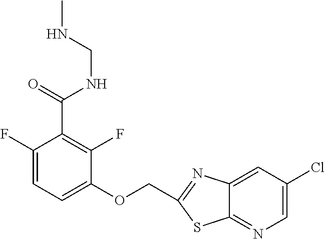 [SMILES: CNCNC(=O)c1c(F)ccc(OCc2nc3cc(Cl)cnc3s2)c1F] | 3-[(6-chlorothiazolo[5,4-b]pyridin-2-yl)methoxy-2,6- difluoro-N-(methylaminomethyl)benzamide |

Other compounds of Formula (I) and compounds of Formula (II) may be prepared according to the General Methods or Schemes previously described herein and/or by reference to the Representative Examples that follow.

### Representative Example 1: Synthesis of 2, 6-difluoro-3-[(2-hydroxyethyl){4-[4-(trifluoromethoxy)phenoxy]butyl}amino]benzamide

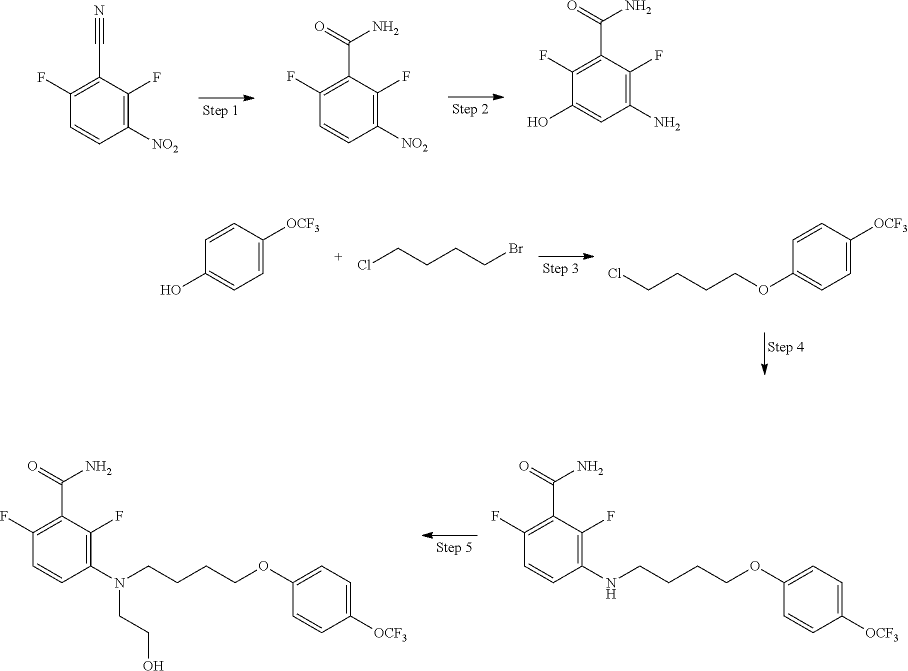

Step 1: 2,6-difluoro-3-nitro-benzonitrile (3.1 g) was dissolved in sulfuric acid (3 mL) and heated to 90° C. for 20 minutes. The solution was poured onto ice and the precipitated white solid was collected by suction filtration. LCMS showed complete conversion to a product with a weak [M+NH₄]⁺ ion (m/z=220). The filter cake was dissolved in THF (25 mL) and dried over MgSO₄. The yellow solution of 2,6-difluoro-3-nitro-benzamide was then used in the next step without further purification.

Step 2: The THF solution of 2,6-difluoro-3-nitro-benzamide from Step 1 was treated with palladium on carbon (100 mg) and covered in an atmosphere of hydrogen at balloon pressure after evacuating the flask three times. The reaction mixture was stirred at room temperature for 70 h. The mixture was filtered to remove the charcoal then concentrated to dryness. The orange residue was recrystallised from isopropanol to yield the target 3-amino-2,6-difluorobenzamide as small tan crystals (1.2 g, 41%).

¹H NMR (400 MHz, DMSO) δ 7.99 (s, 1H), 7.67 (s, 1H), 6.85-6.70 (m, 2H), 5.05 (br s, 2H).

Step 3: 1-Bromo-4-chloro-butane (1.5 mL, 13.5 mmol) and 4-(trifluoromethoxy)phenol (1.45 mL, 11.2 mmol) were dissolved in DMA (5 mL) and treated with potassium carbonate (2.3 g, 1.5 eq). The mixture was stirred at room temperature for 20 h. After this time the mixture was diluted with EtOAc (40 mL) and washed with water (3×40 mL). The organic phase was dried over MgSO₄ and purified by silica chromatography (0%-30% gradient of EtOAc in heptane) to yield 1-(4-chlorobutoxy)-4-(trifluoromethoxy)benzene as a clear liquid (2.36 g, 78%).

Step 4: A solution of 3-amino-2,6-difluorobenzamide (0.70 g, 4.0 mmol) and 1-(4-chlorobutoxy)-4-(trifluoromethoxy)benzene (1.2 g, 1.2 eq) in DMA (1.0 mL) was treated with potassium carbonate (620 mg, 1.2 eq) and catalytic sodium iodide (50 mg, 0.33 mmol). The suspension was heated at 90° C. for 16 hours. After this time the mixture was diluted with EtOAc (100 mL) and washed with water (100 mL) and brine (3×60 mL). The organic layer was dried over MgSO₄ and concentrated. The residue was purified by reverse-phase chromatography (5-100% gradient of MeCN/water) to yield the target 2,6-difluoro-3-[4-[4-(trifluoromethoxy)phenoxy]butylamino]benzamide (890 mg, 54%) as a light brown solid and the by-product 3-[bis[4-[4-(trifluoromethoxy)phenoxy]butyl]amino]-2,6-difluoro-benzamide (411 mg, 16%) as a yellow oil.

Step 5: A mixture of 2,6-difluoro-3-[4-[4-(trifluoromethoxy)phenoxy]butylamino]benzamide (100 mg, 0.247 mmol), K₂CO₃ (50 mg, 1.5 eq) and 2-bromoethanol (65 mg, 5 eq) in dry DMA (0.5 mL) was heated at 90° C. for 64 h. After this time the reaction was cooled and quenched by addition of water (1 mL). The mixture was diluted with EtOAc (2 mL). The organic phase was washed with water (2×1 mL), dried over MgSO₄ and concentrated in vacuo. The residue was taken up in DMSO and purified by reverse phase MPLC (5-100% gradient of MeCN in water) to yield the target as a brown tacky gum (65 mg, 59%).

Prepared by analogous procedures were, for example, 3-[(3-cyanopropyl){4-[4-(trifluoromethoxy)phenoxy]butyl}amino]-2,6-difluorobenzamide and 2,6-difluoro-3-(methyl {4-[4-(trifluoromethoxy)phenoxy]butyl}amino)benzamide.

### Representative Example 2: Synthesis of 2,6-difluoro-3-{4-[4-(trifluoromethoxy)phenoxy]butoxy}benzamide

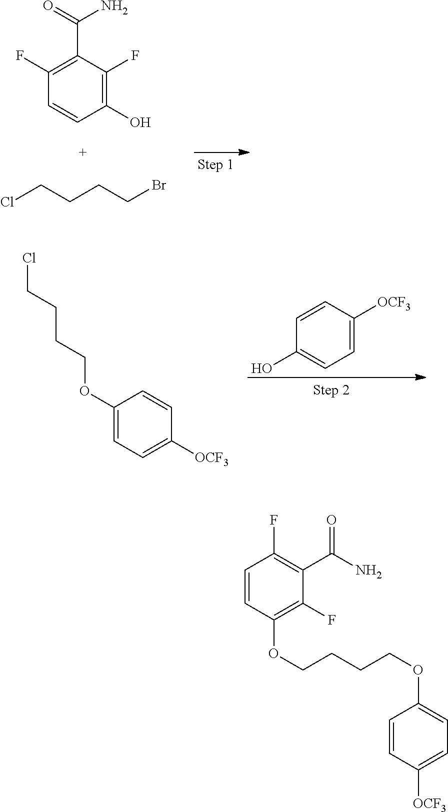 [SMILES: NC(=O)c1c(F)ccc(O)c1F ; ClCCCCBr ; FC(F)(F)Oc1ccc(OCCCCCl)cc1 ; Oc1ccc(OC(F)(F)F)cc1 ; NC(=O)c1c(F)ccc(OCCCCOc2ccc(OC(F)(F)F)cc2)c1F]

Step 1: A solution of 2,6-difluoro-3-hydroxy-benzamide (2.5 g, prepared as described in WO2012/142671) in DMA (10 mL) was treated with potassium carbonate (6 g, 3 eq) and 1-bromo-4-chloro-butane (1.66 mL, 1 eq) and stirred at 60° C. for 6 h.

Step 2: To the above suspension was added 4-(trifluoromethoxy)phenol (2.25 mL, 1.2 eq) and the mixture was stirred at 60° C. for 22 h, then 70° C. for 68 h. The mixture was quenched with water (100 mL) and washed with CH₂Cl₂ (3×100 mL). The combined organic layers were dried over MgSO₄ and concentrated to dryness. The residue was purified by reverse-phase MPLC (50-100% gradient of MeOH in water). The product was freeze-dried from acetonitrile/water to obtain the title compound as a white fluffy solid (2.19 g, 37%).

Prepared by analogous procedures were, for example, 3-{[5-(4-chlorophenoxy)pentyl]oxy}-2,6-difluorobenzamide and 2,6-difluoro-3-(4-{[6-(trifluoromethyl)pyridazin-3-yl]oxy}butoxy)benzamide.

Similarly prepared was, for example, 2,6-difluoro-3-(2-{2-[4-(trifluoromethoxy)phenoxy]ethoxy}ethoxy)benzamide, using 2-(2-chloroethoxy)ethanol in place of 1-bromo-4-chloro-butane and then converting the free hydroxyl group to an alkyl chloride by means of thionyl chloride, then concentrating and resuspending in DMA prior to Step 2. Also similarly prepared were, for example, 3-{4-[(4-chlorophenyl)thio]butoxy}-2,6-difluorobenzamide, 2,6-difluoro-3-{4-[4-(pentafluoro-lambda-6-sulfanyl)phenoxy]butoxy}benzamide and 2,6-difluoro-3-(4-{[4-(trifluoromethoxy)phenyl]amino}butoxy)benzamide by using 4-chlorothiophenol, 4-pentafluorosulfanylphenol and 4-(trifluoromethoxy)aniline, respectively, in place of 4-(trifluoromethoxy)phenol.

### Representative Example 3: Synthesis of 2, 6-difluoro-3-(nonyloxy)benzenecarboximidamide

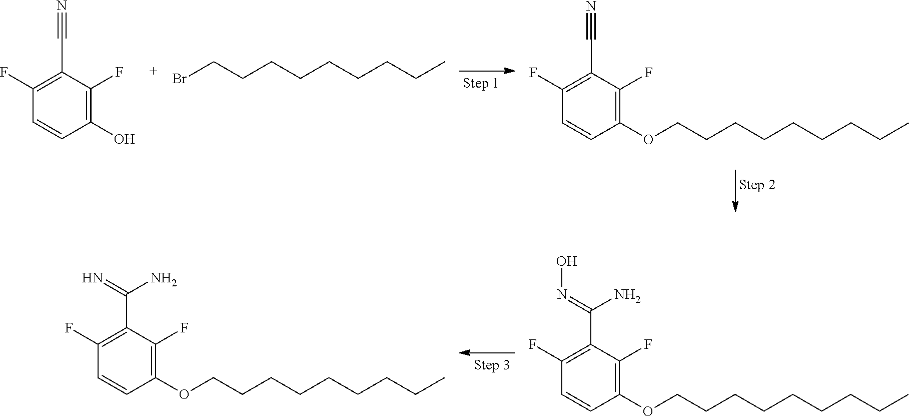

Step 1: A sample of 2,6-difluoro-3-hydroxy-benzonitrile (300 mg) was dissolved in DMA (1 mL) and treated with potassium carbonate (400 mg, 1.5 eq) and 1-bromononane (442 μL, 1.2 eq). The mixture was stirred at 70° C. for 1 h. The mixture was diluted with water (20 mL) and washed with EtOAc (3×20 mL). The combined organic phases were dried over MgSO₄ and concentrated to remove most of the volatiles. The colorless DMA solution thus obtained was used in the next step without further purification.

Step 2: The above solution of 2,6-difluoro-3-nonoxy-benzonitrile was dissolved in ethanol (5 mL) and treated with 50% w/v hydroxylamine in water (355 μL, 3 eq). The solution was stirred at 70° C. for 16 h. The mixture was cooled, diluted with water (25 mL) and washed with dichloromethane (3×40 mL). The combined organic phases were dried over MgSO₄ and purified by silica chromatography (0-40% gradient of EtOAc in heptanes) to yield 2,6-difluoro-N′-hydroxy-3-(nonyloxy)benzenecarboximidamide as a white fluffy solid (366 mg, 60%) after freeze-drying from acetonitrile/water.

Step 3: Potassium formate solution was prepared by treating a solution of formic acid (377 μL, 10 mmol) in methanol (1 mL) with potassium carbonate (691 mg, 5 mmol) and sonicating until the evolution of gas had ceased.

A solution of 2,6-difluoro-N′-hydroxy-3-nonoxy-benzamidine (290 mg) in AcOH (1 mL) was treated with acetic anhydride (100 μL, 1.15 eq) at room temperature. After 30 minutes, the above potassium formate solution was added, followed by a catalytic amount of 10% Pd on carbon. The mixture was stirred at room temperature for 2 h, whereupon a second batch of the above potassium formate solution was prepared and added. The mixture was then stirred at room temperature overnight. The mixture was filtered and washed with ethanol. The filtrate was concentrated, resuspended in anhydrous methanol and then filtered again to remove the undissolved KCl. The filtrate was concentrated, resuspended in dichloromethane and filtered again to remove the undissolved crystals. The filtrate was purified by silica chromatography (0-70% gradient of MeOH in DCM containing 1% TEA) to yield the title compound as a white solid (158 mg, 57%).

### Representative Example 4: Synthesis of 2, 6-difluoro-3-({5-[4-(trifluoromethyl)phenyl]pentyl}oxy)benzamide

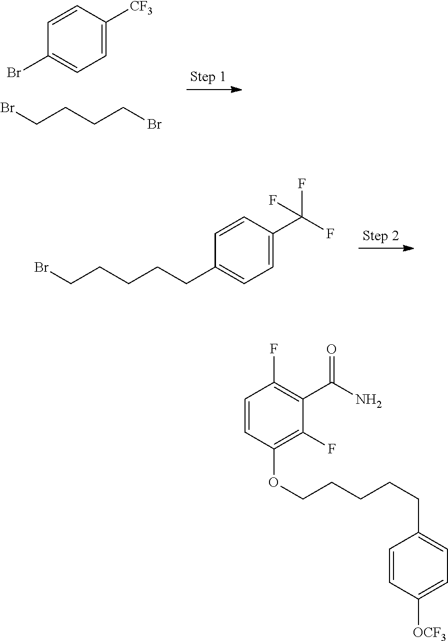

Step 1: Magnesium turnings (420 mg) were added to a solution of 1-bromo-4-(trifluoromethyl)benzene (3.5 g, 16 mmol) in THF (20 mL) at room temperature. An iodine crystal was added and allowed to stand for 5 minutes. After this, stirring was commenced and the mixture began to heat at reflux under its own exotherm. After the exothermic reaction subsided, the mixture was stirred at reflux for a further 30 minutes before cooling to room temperature.

In a separate vessel, a green solution of copper(I) bromide (100 mg, 0.7 mmol) and lithium bromide (120 mg, 1.4 mmol) in THF (25 mL) was treated by rapid addition of 1,5-dibromopentane (9 g, 2.5 eq). The mixture was stirred at room temperature for 30 minutes then warmed to 40° C. The freshly prepared Grignard solution from above was then added over a period of approximately 2 minutes and the mixture was stirred at 55° C. for 3 h and subsequently at room temperature for 16 h. Saturated ammonium chloride solution (20 mL) and ice (20 mL) were added carefully to the reaction mixture. The mixture was washed three times with ethyl acetate and the combined organic phases were dried over MgSO₄ and concentrated. The crude product was purified by silica chromatography (0-100% gradient of EtOAc in hexanes) to yield 1-bromo-4-(trifluoromethyl)benzene as a clear liquid that partially crystallised upon standing (500 mg).

Step 2: A solution of 2,6-difluoro-3-hydroxy-benzamide (100 mg) and 1-(5-bromopentyl)-4-(trifluoromethyl)benzene (300 mg) in DMA (0.7 mL) was treated with potassium carbonate (120 mg, 1.5 eq) and heated at 75° C. with stirring for 3.5 h.

The mixture was diluted with water (1 mL) and then washed with EtOAc (4×1 mL).

The combined organic layers were filtered through cotton wool, concentrated and purified by reverse-phase MPLC (5-100% gradient of MeCN in water). The product-containing fractions were freeze-dried to yield the title ether as a white solid (70 mg, 31%).

### Representative Example 5: Synthesis of 2, 6-difluoro-3-({1-[4-(trifluoromethoxy)phenyl]-1H-pyrazol-3-yl}methoxy)benzamide

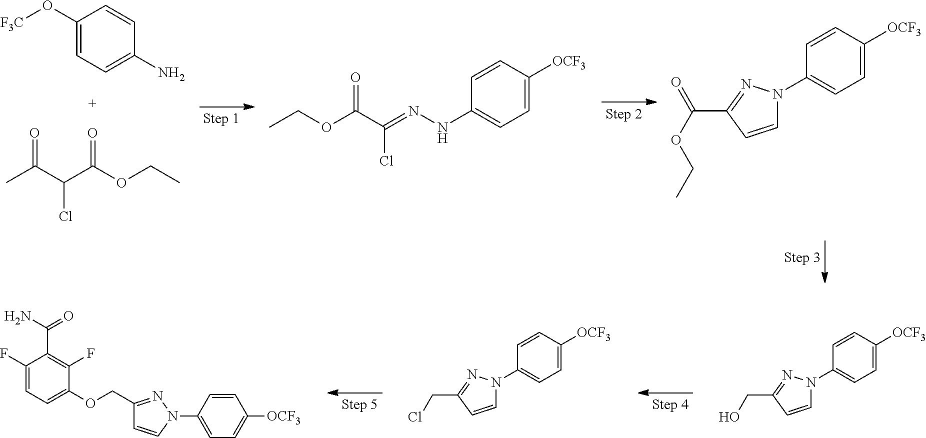

Step 1: A solution of 4-(trifluoromethoxy)aniline (2 g) in 5 M HCl (4 mL) and acetone (10 mL) was cooled in ice/acetone. To this, a solution of NaNO₂ (860 mg, 1.1 eq) in water (1 mL) was slowly added at such a rate that the temperature stayed between −10 and 0° C. The resulting solution was stirred for 1 h before adding it to a cold mixture of NaOAc (2.8 g, 3 eq), ethanol (30 ml) and ethyl 2-chloroacetoacetate (1.86 g, 1 eq). After stirring the resulting mixture for 1 hour the precipitate was collected by filtration, washed with water and dried in vacuo to give ethyl (2Z)-2-chloro-2-[[4-(trifluoromethoxy)phenyl]hydrazono]acetate (2.87 g, 82%).

Step 2: A mixture of ethyl 1-[4-(trifluoromethoxy)phenyl]pyrazole-3-carboxylate (2.87 g), bicyclo[2.2.1]hepta-2,5-diene (5 mL, 5 eq) and triethylamine (3.8 mL, 3 eq) in toluene (20 mL) was stirred at 70° C. for 0.5 h. The mixture was cooled and filtered, the filter cake was washed with toluene and the filtrate was concentrated under vacuum. The residue was heated at reflux in xylenes (30 mL) for 1 h. The mixture was partially concentrated under vacuum then purified by silica chromatography (0-50% gradient of EtOAc in heptanes) to yield ethyl 1-[4-(trifluoromethoxy)phenyl]pyrazole-3-carboxylate as an orange oil (1.89 g, 68%).

Step 3: A solution of ethyl 1-[4-(trifluoromethoxy)phenyl]pyrazole-3-carboxylate (1.89 g) in THF (20 mL) was cooled in ice/water and cautiously treated with lithal (260 mg, 1.1 eq). The ice bath was withdrawn and the mixture was stirred at room temperature under nitrogen for 75 minutes. The mixture was cooled in ice/water and quenched with ice, then diluted with THF (30 mL) and a saturated solution of sodium potassium tartrate (Rochelle salt). The aqueous layer was washed again with THF (30 mL) and the combined organic extracts were dried over MgSO₄ and concentrated to yield [1-[4-(trifluoromethoxy)phenyl]pyrazol-3-yl]methanol as an orange oil, which crystallised on standing (1.53 g, 94% crude).

Step 4: A solution of crude [1-[4-(trifluoromethoxy)phenyl]pyrazol-3-yl]methanol (1.53 g) in dichloromethane (10 mL) was treated at room temperature with thionyl chloride (3 mL, 7 eq) and catalytic DMA (10 drops). The mixture initially boiled under its own exotherm. After 0.5 h the mixture was quenched with ice (20 mL). The aqueous layer was washed with dichloromethane (2×25 mL) and the combined organic layers were dried over MgSO₄ and concentrated. The residue was purified by silica chromatography (0-50% gradient of EtOAc in heptanes) to yield 3-(chloromethyl)-1-[4-(trifluoromethoxy)phenyl]pyrazole as an orange liquid (1.41 g, 86%).

Step 5: A mixture containing 2,6-difluoro-3-hydroxy-benzamide (150 mg), 3-(chloromethyl)-1-[4-(trifluoromethoxy)phenyl]pyrazole (240 mg, 1.2 eq) and potassium carbonate (180 mg, 1.5 eq) in DMA (0.6 mL) was stirred at 90° C. for 19 h. The mixture was diluted with water (5 mL) and washed with a combination of dichloromethane, THF and EtOAc (20 mL). The combined organic extracts were partially concentrated under vacuum and the residue was purified by reverse-phase MPLC (5-100% gradient of MeCN in water) then freeze-dried to yield the title compound as a fluffy white solid (159 mg, 44%).

### Representative Example of prodrug formation: Synthesis of (R)-4-(2-(5-bromo-4-(4-(trifluoromethyl)phenyl)oxazol-2-yl)-2-(3-carbamoyl-2, 4-difluorophenoxy)ethoxy)-4-oxobutanoic acid

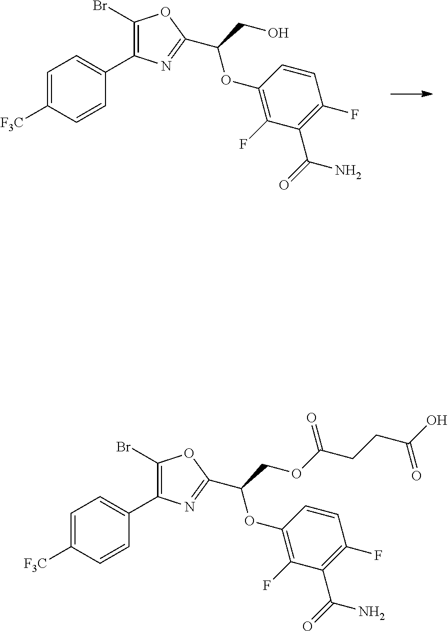 [SMILES: NC(=O)c1c(F)ccc(O[C@H](CO)c2nc(-c3ccc(C(F)(F)F)cc3)c(Br)o2)c1F ; NC(=O)c1c(F)ccc(O[C@H](COC(=O)CCC(=O)O)c2nc(-c3ccc(C(F)(F)F)cc3)c(Br)o2)c1F]

To a solution of (R)-3-(1-(5-bromo-4-(4-(trifluoromethyl)phenyl)oxazol-2-yl)-2-hydroxyethoxy)-2,6-difluorobenzamide (0.40 g, 0.79 mmol) in pyridine (10 mL) was added DMAP (catalytic) and succinic anhydride (0.08 g, 0.79 mmol). The resulting reaction mixture was allowed to stir at room temperature for 16 h. The reaction mixture was cooled to 0° C., adjusted to pH 4-5 by drop-wise addition of 6 M HCl and the product extracted into EtOAc (2×50 mL). The combined organic layers were dried over anhydrous Na₂SO₄, filtered and concentrated under reduced pressure to afford (R)-4-(2-(5-bromo-4-(4-(trifluoromethyl)phenyl)oxazol-2-yl)-2-(3-carbamoyl-2,4-difluorophenoxy)ethoxy)-4-oxobutanoic acid (0.40 g, 84%).

### Representative Example of salt formation: Synthesis of (R)-4-(2-(5-bromo-4-(4-(trifluoromethyl)phenyl)oxazol-2-yl)-2-(3-carbamoyl-2, 4-difluorophenoxy)ethoxy)-4-oxobutanoic acid arginine salt

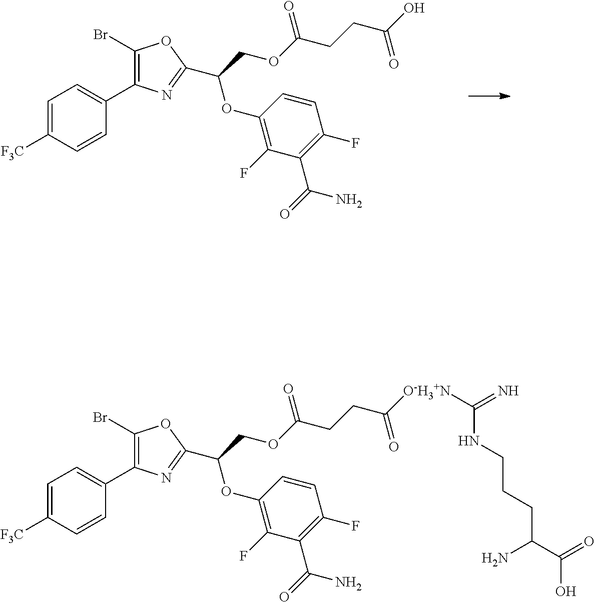

To a solution of (R)-4-(2-(5-bromo-4-(4-(trifluoromethyl)phenyl)oxazol-2-yl)-2-(3-carbamoyl-2,4-difluorophenoxy)ethoxy)-4-oxobutanoic acid (0.30 g, 0.49 mmol) in methanol:DCM (1:1, 8 mL) was added L-arginine (0.086 g, 0.49 mmol). The resulting reaction mixture was stirred at room temperature for 1 h. After completion of the reaction (TLC monitoring) the mixture was concentrated under reduced pressure and the residue was triturated with diethyl ether to afford (R)-4-(2-(5-bromo-4-(4-(trifluoromethyl)phenyl)oxazol-2-yl)-2-(3-carbamoyl-2,4-difluorophenoxy)ethoxy)-4-oxobutanoic acid L-arginine salt as an off white solid (0.30 g, 78%).

Similarly prepared was 4-(2-(5-bromo-4-(4-(trifluoromethyl)phenyl)oxazol-2-yl)-2-(3-carbamoyl-2,4-difluorophenoxy)ethoxy)-4-oxobutanoic acid L-arginine salt.

### Representative Example of Mannich Base Formation: Synthesis of 3-[(6-chlorothiazolo[5, 4-b]pyridin-2-yl)methoxy]-2, 6-difluoro-N-(methylaminomethyl)benzamide

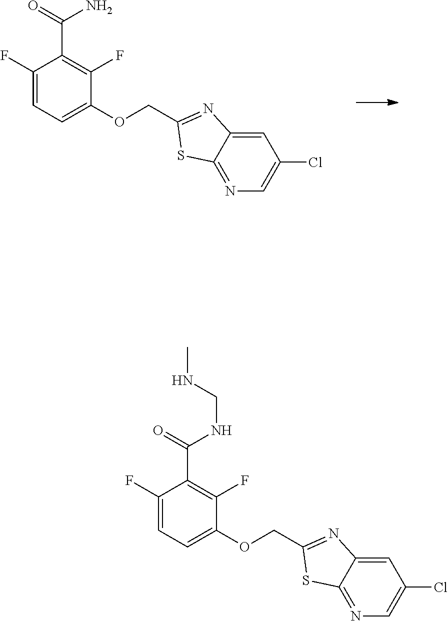 [SMILES: NC(=O)c1c(F)ccc(OCc2nc3cc(Cl)cnc3s2)c1F ; CNCNC(=O)c1c(F)ccc(OCc2nc3cc(Cl)cnc3s2)c1F]

An ethanolic solution of methylamine (1.26 mmol) and formaldehyde (1.26 mmol) was stirred for 15 min at room temperature followed by addition of 3-[(6-chlorothiazolo[5,4-b]pyridin-2-yl)methoxy]-2,6-difluoro-benzamide (0.10 g, 0.21 mmol). The resulting reaction mixture was stirred at 80° C. for 16 h. The reaction mass was then concentrated under reduced pressure and finally triturated with n-pentane to obtain the title product.

Compounds 104-121 of Formula (I) were characterised using a combination of LCMS and ¹H NMR data provided in Table 1 and 1a.

| Cpd | | |
|---|---|---|
| No | Structure | Name (ACD) |
| 104 | 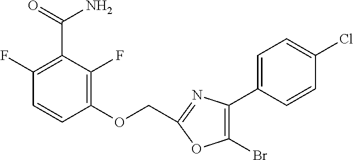 [SMILES: NC(=O)c1c(F)ccc(OCc2nc(-c3ccc(Cl)cc3)c(Br)o2)c1F] | 3-{[5-bromo-4-(4-chlorophenyl)-1,3-oxazol-2- yl]methoxy}-2,6-difluorobenzamide |
| 105 | 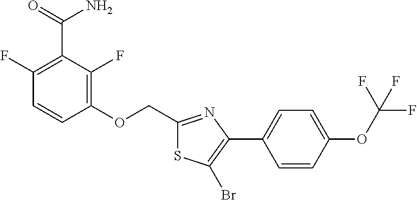 [SMILES: NC(=O)c1c(F)ccc(OCc2nc(-c3ccc(OC(F)(F)F)cc3)c(Br)s2)c1F] | 3-({5-bromo-4-[4-(trifluoromethoxy)phenyl]-1,3- thiazol-2-yl}methoxy)-2,6-difluorobenzamide |
| 106 | 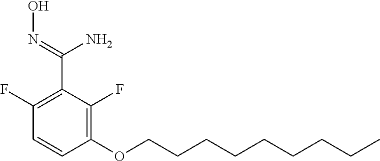 [SMILES: CCCCCCCCCOc1ccc(F)c(/C(N)=N/O)c1F] | 2,6-difluoro-N′-hydroxy-3- (nonyloxy)benzenecarboximidamide |
| 107 | 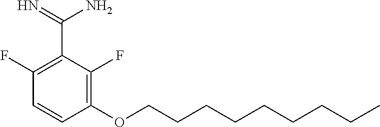 [SMILES: CCCCCCCCCOc1ccc(F)c(C(=N)N)c1F] | 2,6-difluoro-3-(nonyloxy)benzenecarboximidamide |
| 108 | 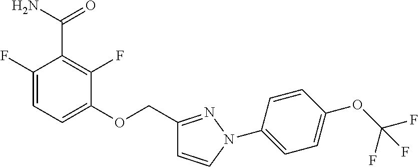 [SMILES: NC(=O)c1c(F)ccc(OCc2ccn(-c3ccc(OC(F)(F)F)cc3)n2)c1F] | 2,6-difluoro-3-({1-[4-(trifluoromethoxy)phenyl]-1H- pyrazol-3-yl}methoxy)benzamide |
| 109 | 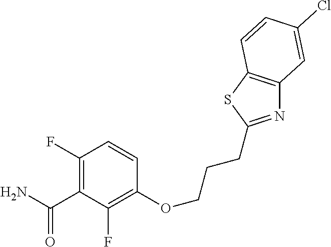 [SMILES: NC(=O)c1c(F)ccc(OCCCc2nc3cc(Cl)ccc3s2)c1F] | 3-[3-(5-chloro-1,3-benzothiazol-2-yl)propoxy]-2,6- difluorobenzamide |
| 110 | 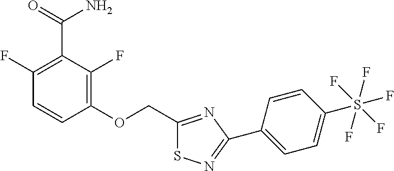 [SMILES: NC(=O)c1c(F)ccc(OCc2nc(-c3ccc(S(F)(F)(F)(F)F)cc3)ns2)c1F] | 2,6-difluoro-3-({3-[4-(pentafluoro-lambda~6~- sulfanyl)phenyl]-1,2,4-thiadiazol-5- yl}methoxy)benzamide |
| 111 | 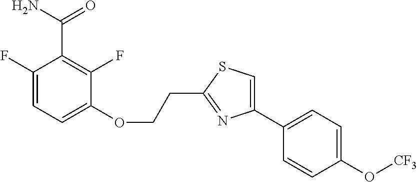 [SMILES: NC(=O)c1c(F)ccc(OCCc2nc(-c3ccc(OC(F)(F)F)cc3)cs2)c1F] | 2,6-difluoro-3-(2-{4-[4-(trifluoromethoxy)phenyl]-1,3- thiazol-2-yl}ethoxy)benzamide |
| 112 | 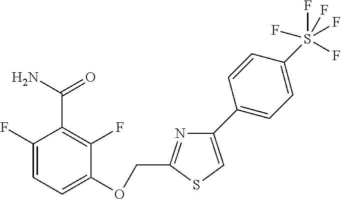 [SMILES: NC(=O)c1c(F)ccc(OCc2nc(-c3ccc(S(F)(F)(F)(F)F)cc3)cs2)c1F] | 2,6-difluoro-3-({4-[4-(pentafluoro-lambda~6~- sulfanyl)phenyl]-1,3-thiazol-2-yl}methoxy)benzamide |
| 113 | 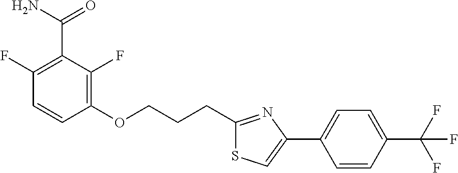 [SMILES: NC(=O)c1c(F)ccc(OCCCc2nc(-c3ccc(C(F)(F)F)cc3)cs2)c1F] | 2,6-difluoro-3-(3-{4-[4-(trifluoromethyl)phenyl]-1,3- thiazol-2-yl}propoxy)benzamide |
| 114 | 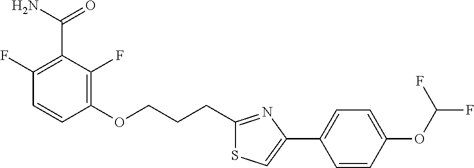 [SMILES: NC(=O)c1c(F)ccc(OCCCc2nc(-c3ccc(OC(F)F)cc3)cs2)c1F] | 3-(3-{4-[4-(difluoromethoxy)phenyl]-1,3-thiazol-2- yl}propoxy)-2,6-difluorobenzamide |
| 115 | 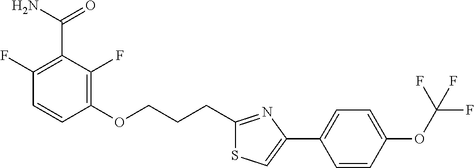 [SMILES: NC(=O)c1c(F)ccc(OCCCc2nc(-c3ccc(OC(F)(F)F)cc3)cs2)c1F] | 2,6-difluoro-3-(3-{4-[4-(trifluoromethoxy)phenyl]-1,3- thiazol-2-yl}propoxy)benzamide |
| 116 | 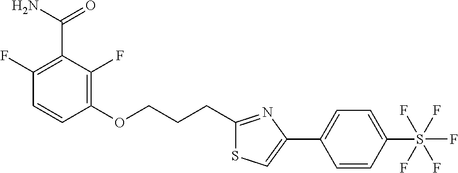 [SMILES: NC(=O)c1c(F)ccc(OCCCc2nc(-c3ccc(S(F)(F)(F)(F)F)cc3)cs2)c1F] | 2,6-difluoro-3-(3-{4-[4-(pentafluoro-lambda~6~- sulfanyl)phenyl]-1,3-thiazol-2-yl}propoxy)benzamide |
| 117 | 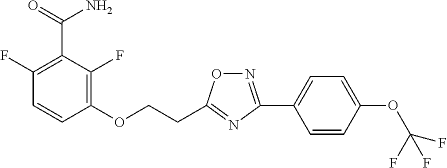 [SMILES: NC(=O)c1c(F)ccc(OCCc2nc(-c3ccc(OC(F)(F)F)cc3)no2)c1F] | 2,6-difluoro-3-(2-{3-[4-(trifluoromethoxy)phenyl]- 1,2,4-oxadiazol-5-yl}ethoxy)benzamide |
| 118 | 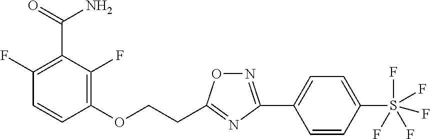 [SMILES: NC(=O)c1c(F)ccc(OCCc2nc(-c3ccc(S(F)(F)(F)(F)F)cc3)no2)c1F] | 2,6-difluoro-3-(2-{3-[4-(pentafluoro-lambda~6~- sulfanyl)phenyl]-1,2,4-oxadiazol-5- yl}ethoxy)benzamide |
| 119 | 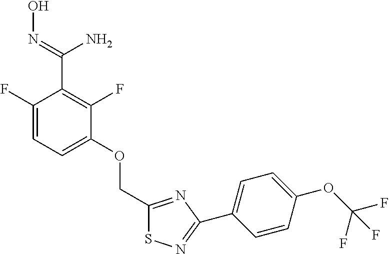 [SMILES: N/C(=N\O)c1c(F)ccc(OCc2nc(-c3ccc(OC(F)(F)F)cc3)ns2)c1F] | 2,6-difluoro-N′-hydroxy-3-({3-[4- (trifluoromethoxy)phenyl]-1,2,4-thiadiazol-5- yl}methoxy)benzenecarboximidamide |
| 120 | 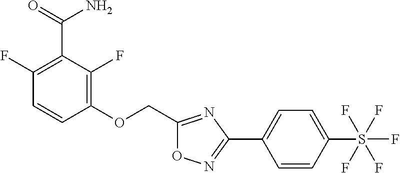 [SMILES: NC(=O)c1c(F)ccc(OCc2nc(-c3ccc(S(F)(F)(F)(F)F)cc3)no2)c1F] | 2,6-difluoro-3-({3-[4-(pentafluoro-lambda~6~- sulfanyl)phenyl]-1,2,4-oxadiazol-5- yl}methoxy)benzamide |
| 121 | 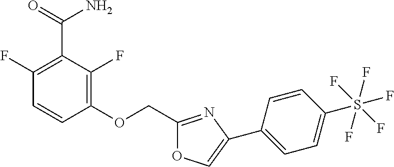 [SMILES: NC(=O)c1c(F)ccc(OCc2nc(-c3ccc(S(F)(F)(F)(F)F)cc3)co2)c1F] | 2,6-difluoro-3-({4-[4-(pentafluoro-lambda~6~- sulfanyl)phenyl]-1,3-oxazol-2-yl}methoxy)benzamide |

TABLE 1
Characterisation of compounds of Formula (I) by LCMS and ¹H NMR

| | | LCMS | |
|---|---|---|---|
| | | m/z = | |
| No. | Name | [M + H]⁺ | ¹H NMR |
| 104 | 3-{[5-bromo-4-(4- | 444.9 | ¹H NMR (400 MHz, DMSO) δ 8.15 (br |
| | chlorophenyl)-1,3-oxazol-2- | | s, 1H), 7.90 (d, J = 8.8 Hz, 2H), 7.88 (br |
| | yl]methoxy}-2,6- | | s, 1H), 7.59 (d, J = 8.8 Hz, 2H), 7.39 (td, |
| | difluorobenzamide | | J = 9.3, 5.2 Hz, 1H), 7.13 (td, J = 9.0, |
| | | | 1.9 Hz, 1H), 5.39 (s, 2H). |
| 105 | 3-({5-bromo-4-[4- | 509.1 | 1H NMR (400 MHz, Acetone-d6) δ |
| | (trifluoromethoxy)phenyl]-1,3- | | 8.12 (d, J = 9.0 Hz, 2H), 7.59-7.43 (m, 3H), |
| | thiazol-2-yl}methoxy)-2,6- | | 7.45-7.37 (m, 1H), 7.20 (s, 1H), |
| | difluorobenzamide | | 7.03 (ddd, J = 9.2, 8.6, 2.1 Hz, 1H), 5.55 (s, |
| | | | 2H). |
| 106 | 2,6-difluoro-N′-hydroxy-3- | 315.2 | 1H NMR major tautomer (400 MHz, |
| | (nonyloxy)benzenecarboximidamide | | Acetone, 70° C.) δ 8.95 (br s, 1H), |
| | | | 7.17 (td, J = 9.2, 5.2 Hz, 1H), 6.93 (td, J = 9.0, |
| | | | 2.1 Hz, 1H), 5.54 (br s, 2H), 4.06 (t, |
| | | | J = 6.5 Hz, 2H), 1.83-1.74 (m, 2H), |
| | | | 1.53-1.43 (m, 2H), 1.42-1.23 (m, |
| | | | 10H), 0.88 (t, J = 6.9 Hz, 3H). |
| 107 | 2,6-difluoro-3- | 222.9 | 1H NMR (400 MHz, CDCl3) δ 6.99 (td, |
| | (nonyloxy)benzenecarboximidamide | | J = 9.1, 5.1 Hz, 1H), 6.89 (td, J = 9.1, |
| | | | 1.9 Hz, 1H), 5.08 (br s, 3H, |
| | | | exchangeable), 4.00 (t, J = 6.6 Hz, 2H), |
| | | | 1.84-1.74 (m, 2H), 1.51-1.40 (m, |
| | | | 2H), 1.39-1.20 (m, 10H), 0.88 (t, J = 6.9 Hz, |
| | | | 3H). |
| 108 | 2,6-difluoro-3-({1-[4- | 414.0 | ¹H NMR (400 MHz, CDCl₃) δ 7.88 (d, J = 2.5 Hz, |
| | (trifluoromethoxy)phenyl]-1H- | | 1H), 7.70 (d, J = 9.2 Hz, 2H), |
| | pyrazol-3- | | 7.34-7.29 (m, 2H), 7.19 (td, J = 9.1, |
| | yl}methoxy)benzamide | | 5.1 Hz, 1H), 6.86 (td, J = 9.2, 2.1 Hz, |
| | | | 1H), 6.58 (d, J = 2.5 Hz, 1H), 5.96 (br s, |
| | | | 1H), 5.87 (br s, 1H), 5.23 (s, 2H). |
| 109 | 3-[3-(5-chloro-1,3- | 383.0 | 1H NMR (400 MHz, CD3CN) δ 8.00 (d, |
| | benzothiazol-2-yl)propoxy]- | | J = 2.0 Hz, 1H), 7.96 (d, J = 8.6 Hz, |
| | 2,6-difluorobenzamide | | 1H), 7.44 (dd, J = 8.6, 2.1 Hz, 1H), |
| | | | 7.20-7.10 (m, 1H), 7.02-6.94 (m, 1H), |
| | | | 6.68 (s, 1H), 6.43 (s, 1H), 4.20 (t, J = 6.2 Hz, |
| | | | 2H), 3.33 (t, J = 7.5 Hz, 2H), |
| | | | 2.44-2.28 (m, 2H). |
| 110 | 2,6-difluoro-3-({3-[4- | 474.0 | ¹H NMR (400 MHz, DMSO) δ 8.43 (d, |
| | (pentafluoro-lambda~6~- | | J = 8.8 Hz, 2H), 8.17 (br s, 1H), 8.11 (d, |
| | sulfanyl)phenyl]-1,2,4- | | J = 9.0 Hz, 2H), 7.89 (br s, 1H), |
| | thiadiazol-5- | | 7.46 (td, J = 9.4, 5.2 Hz, 1H), 7.15 (td, J = 9.1, |
| | yl}methoxy)benzamide | | 1.9 Hz, 1H), 5.85 (s, 2H). |
| 111 | 2,6-difluoro-3-(2-{4-[4- | 445.2 | 1H NMR (400 MHz, CDCl3) δ |
| | (trifluoromethoxy)phenyl]-1,3- | | 7.97-7.91 (m, 2H), 7.43 (s, 1H), |
| | thiazol-2-yl}ethoxy)benzamide | | 7.35-7.28 (m, 2H), 7.10 (td, J = 9.1, 5.1 Hz, 1H), |
| | | | 6.97-6.88 (m, 1H), 5.98 (d, J = 19.8 Hz, |
| | | | 2H), 4.50 (t, J = 6.4 Hz, 2H), 3.60 (t, |
| | | | J = 6.4 Hz, 2H). |
| 112 | 2,6-difluoro-3-({4-[4- | 473.0 | 1H NMR (400 MHz, MeOD) δ |
| | (pentafluoro-lambda~6~- | | 8.19-8.10 (m, 3H), 7.96-7.86 (m, 2H), |
| | sulfanyl)phenyl]-1,3-thiazol-2- | | 7.38 (td, J = 9.2, 5.1 Hz, 1H), 7.02 (td, J = 9.1, |
| | yl}methoxy)benzamide | | 2.1 Hz, 1H), 5.55 (s, 2H). |
| 113 | 2,6-difluoro-3-(3-{4-[4- | 443.1 | ¹H NMR (400 MHz, CDCl₃) δ 7.99 (d, J = 8.0 Hz, |
| | (trifluoromethyl)phenyl]-1,3- | | 2H), 7.66 (d, J = 8.2 Hz, 2H), |
| | thiazol-2- | | 7.46 (s, 1H), 7.02 (td, J = 9.0, 5.1 Hz, |
| | yl}propoxy)benzamide | | 1H), 6.87 (td, J = 9.1, 2.0 Hz, 1H), |
| | | | 5.95 (br s, 1H), 5.85 (br s, 1H), 4.16 (t, J = 6.1 Hz, |
| | | | 2H), 3.29 (t, J = 7.4 Hz, 2H), |
| | | | 2.43-2.33 (m, 2H). |
| 114 | 3-(3-{4-[4- | 441.1 | ¹H NMR (400 MHz, CDCl₃) δ 7.87 (d , J = 8.9 Hz, |
| | (difluoromethoxy)phenyl]-1,3- | | 2H), 7.32 (s, 1H), 7.16 (d, J = 8.9 Hz, |
| | thiazol-2-yl}propoxy)-2,6- | | 2H), 7.02 (td, J = 9.1, 5.1 Hz, |
| | difluorobenzamide | | 1H), 6.87 (td, J = 9.1, 2.0 Hz, 1H), |
| | | | 6.54 (t, J = 73.9 Hz, 1H), 5.95 (br s, 1H), |
| | | | 5.84 (br s, 1H), 4.15 (t, J = 6.1 Hz, 2H), |
| | | | 3.28 (t, J = 7.3 Hz, 2H), 2.46-2.26 (m, |
| | | | 2H). |
| 115 | 2,6-difluoro-3-(3-{4-[4- | 459.1 | ¹H NMR (400 MHz, CDCl₃) δ 8.05 (d, J = 9.0 Hz, |
| | (trifluoromethoxy)phenyl]-1,3- | | 2H), 7.89 (d, J = 8.9 Hz, 2H), |
| | thiazol-2- | | 7.35 (s, 1H), 7.02 (td, J = 9.1, 5.1 Hz, |
| | yl}propoxy)benzamide | | 1H), 6.87 (td, J = 9.1, 2.0 Hz, 1H), |
| | | | 5.95 (br s, 1H), 5.85 (br s, 1H), 4.15 (t, J = 6.1 Hz, |
| | | | 2H), 3.27 (t, J = 7.4 Hz, 2H), |
| | | | 2.37 (tt, J = 7.2, 6.1 Hz, 2H). |
| 116 | 2,6-difluoro-3-(3-{4-[4- | 501.1 | ¹H NMR (400 MHz, CDCl₃) δ 7.96 (d, J = 8.9 Hz, |
| | (pentafluoro-lambda~6~- | | 2H), 7.79 (d, J = 8.9 Hz, 2H), |
| | sulfanyl)phenyl]-1,3-thiazol-2- | | 7.48 (s, 1H), 7.02 (td, J = 9.1, 5.1 Hz, |
| | yl}propoxy)benzamide | | 1H), 6.87 (td, J = 9.1, 2.0 Hz, 1H), |
| | | | 5.95 (br s, 1H), 5.83 (br s, 1H), 4.16 (t, J = 6.0 Hz, |
| | | | 2H), 3.30 (t, J = 7.4 Hz, 2H), |
| | | | 2.43-2.34 (m, 2H). |
| 117 | 2,6-difluoro-3-(2-{3-[4- | 430.1 | 1H NMR (400 MHz, CDCl3) δ |
| | (trifluoromethoxy)phenyl]- | | 8.25-8.10 (m, 2H), 7.43-7.32 (m, 2H), |
| | 1,2,4-oxadiazol-5- | | 7.13 (td, J = 9.1, 5.2 Hz, 1H), 6.94 (td, J = 9.1, |
| | yl}ethoxy)benzamide | | 2.0 Hz, 1H), 5.97 (s, 2H), 4.58 (t, J = 6.4 Hz, |
| | | | 2H), 3.50 (t, J = 6.4 Hz, 2H). |
| 118 | 2,6-difluoro-3-(2-{3-[4- | 472.0 | 1H NMR (400 MHz, CDCl3) δ |
| | (pentafluoro-lambda~6~- | | 8.27-8.18 (m, 2H), 7.97-7.87 (m, 2H), |
| | sulfanyl)phenyl]-1,2,4- | | 7.13 (td, J = 9.1, 5.1 Hz, 1H), 6.94 (td, J = 9.1, |
| | oxadiazol-5- | | 2.0 Hz, 1H), 5.99 (s, 2H), 4.59 (t, J = 6.4 Hz, |
| | yl}ethoxy)benzamide | | 2H), 3.52 (t, J = 6.4 Hz, 2H). |
| 119 | 2,6-difluoro-N′-hydroxy-3-({3- | 447.2 | ¹H NMR (400 MHz, DMSO-d₆) δ |
| | [4-(trifluoromethoxy)phenyl]- | | 9.62 (br s, 1H), 8.35 (d, J = 8.9 Hz, 2H), |
| | 1,2,4-thiadiazol-5- | | 7.56 (d, J = 8.0 Hz, 2H), 7.46 (td, J = 9.3, 5.1 Hz, |
| | yl}methoxy)benzenecarboximidamide | | 1H), 7.13 (td, J = 9.1, 1.9 Hz, 1H), |
| | | | 6.01 (s, 2H), 5.83 (s, 2H). |
| 120 | 2,6-difluoro-3-({3-[4- | 458.1 | ¹H NMR (400 MHz, Chloroform-d) δ |
| | (pentafluoro-lambda~6~- | | 8.20 (d, J = 8.9 Hz, 2H), 7.89 (d, J = 9.0 Hz, |
| | sulfanyl)phenyl]-1,2,4- | | 2H), 7.21 (td, J = 9.1, 5.1 Hz, 1H), |
| | oxadiazol-5- | | 6.93 (td, J = 9.0, 2.1 Hz, 1H), 5.99 (br s, |
| | yl}methoxy)benzamide | | 1H), 5.94 (br s, 1H), 5.42 (s, 2H). |
| 121 | 2,6-difluoro-3-({4-[4- | 457.0 | ¹H NMR (400 MHz, Acetone) δ 8.18 (s, |
| | (pentafluoro-lambda~6~- | | 1H), 7.82 (d, J = 8.6 Hz, 2H), |
| | sulfanyl)phenyl]-1,3-oxazol-2- | | 7.80-7.67 (m, 2H), 7.17 (td, J = 9.1, 5.2 Hz, |
| | yl}methoxy)benzamide | | 1H), 6.88 (br s, 1H), 6.82 (td, J = 9.0, |
| | | | 2.0 Hz, 1H), 6.53 (br s, 1H), 5.19 (s, |
| | | | 2H). |

Compounds 122-123 of Formula (Ia) were characterised using a combination of LCMS and ¹H NMR data provided in Table 2.

TABLE 2
Characterisation of compounds of Formula (Ia) by LCMS and ¹H NMR

| | | LCMS | |
|---|---|---|---|
| | | m/z = | |
| No. | Name | [M + H]⁺ | ¹H NMR |
| 122 | 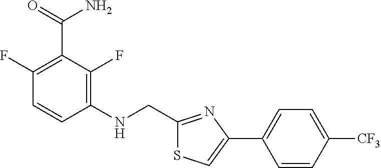 [SMILES: NC(=O)c1c(F)ccc(NCc2nc(-c3ccc(C(F)(F)F)cc3)cs2)c1F] 2,6-difluoro-3-[({4-[4- (trifluoromethyl)phenyl]-1,3-thiazol-2- yl}methyl)amino]benzamide | 414.0 | ¹H NMR (400 MHz, DMSO- d₆) δ 8.23 (s, 1H), 8.17 (d, J = 8.1 Hz, 2H), 8.08 (br s, 1H), 7.81 (d, J = 8.3 Hz, 2H), 7.75 (br s, 1H), 6.88 (t, J = 8.9 Hz, 1H), 6.60 (dt, J = 5.8, 2.9 Hz, 1H), 4.70 (d, J = 6.1 Hz, 2H). |
| 123 | 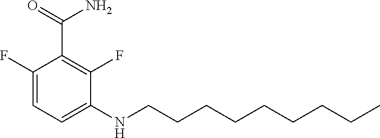 [SMILES: CCCCCCCCCNc1ccc(F)c(C(N)=O)c1F] 2,6-difluoro-3-(nonylamino)benzamide | 299.2 | ¹H NMR (400 MHz, DMSO) δ 8.00 (s, 1H), 7.69 (s, 1H), 6.87 (td, J = 8.9, 1.5 Hz, 1H), 6.66 (td, J = 9.4, 5.5 Hz, 1H), 5.32 (td, J = 5.7, 1.9 Hz, 1H), 3.03 (dd, J = 13.6, 6.5 Hz, 2H), 1.60-1.44 (m, 2H), 1.35-1.19 (m, 12H), 0.86 (t, J = 6.9 Hz, 3H). |

Compounds 124-187 of Formula (II) were characterised using a combination of LCMS and ¹H NMR data provided in Table 3.

TABLE 3
Structures and Characterisation of compounds of Formula (Ia) by LCMS and ¹H NMR
a) Structures

| Cpd | | |
|---|---|---|
| No | Structure | Name (ACD) |
| 124 | 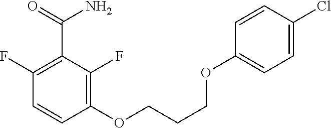 [SMILES: NC(=O)c1c(F)ccc(OCCCOc2ccc(Cl)cc2)c1F] | 3-[3-(4-chlorophenoxy)propoxy]-2,6- difluorobenzamide |
| 125 | 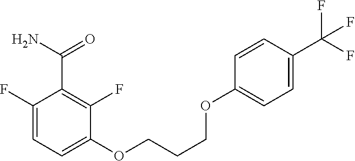 [SMILES: NC(=O)c1c(F)ccc(OCCCOc2ccc(C(F)(F)F)cc2)c1F] | 2,6-difluoro-3-{3-[4- (trifluoromethyl)phenoxy]propoxy}benzamide |
| 126 | 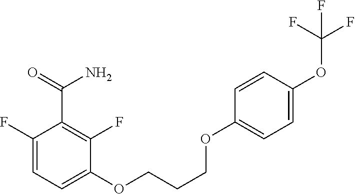 [SMILES: NC(=O)c1c(F)ccc(OCCCOc2ccc(OC(F)(F)F)cc2)c1F] | 2,6-difluoro-3-{3-[4- (trifluoromethoxy)phenoxy]propoxy}benzamide |
| 127 | 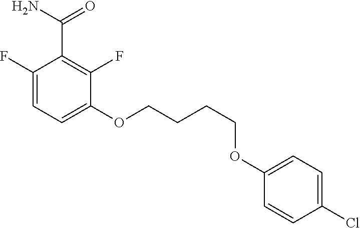 [SMILES: NC(=O)c1c(F)ccc(OCCCCOc2ccc(Cl)cc2)c1F] | 3-[4-(4-chlorophenoxy)butoxy]-2,6-difluoro- benzamide |
| 128 | 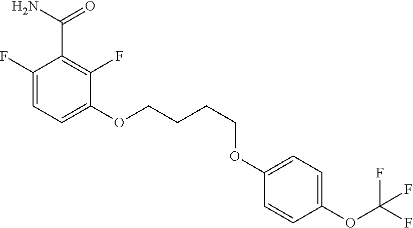 [SMILES: NC(=O)c1c(F)ccc(OCCCCOc2ccc(OC(F)(F)F)cc2)c1F] | 2,6-difluoro-3-{4-[4- (trifluoromethoxy)phenoxy]butoxy}benzamide |
| 129 | 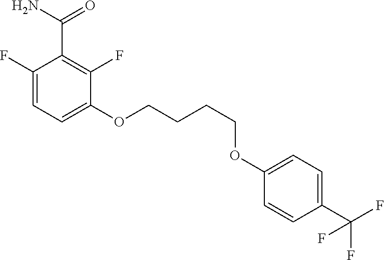 [SMILES: NC(=O)c1c(F)ccc(OCCCCOc2ccc(C(F)(F)F)cc2)c1F] | 2,6-difluoro-3-{4-[4 (trifluoromethyl)phenoxy]butoxy}benzamide |
| 130 | 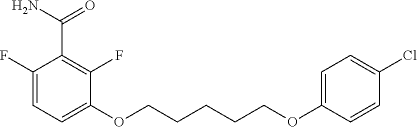 [SMILES: NC(=O)c1c(F)ccc(OCCCCCOc2ccc(Cl)cc2)c1F] | 3-{[5-(4-chlorophenoxy)pentyl]oxy}-2,6- difluorobenzamide |
| 131 | 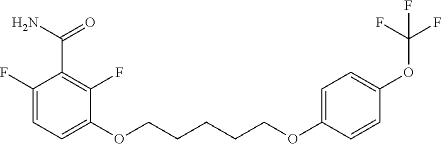 [SMILES: NC(=O)c1c(F)ccc(OCCCCCOc2ccc(OC(F)(F)F)cc2)c1F] | 2,6-difluoro-3-({5-[4- (trifluoromethoxy)phenoxy]pentyl}oxy) benzamide |
| 132 | 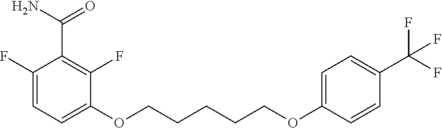 [SMILES: NC(=O)c1c(F)ccc(OCCCCCOc2ccc(C(F)(F)F)cc2)c1F] | 2,6-difluoro-3-({5-[4 (trifluoromethyl)phenoxy]pentyl}oxy) benzamide |
| 133 | 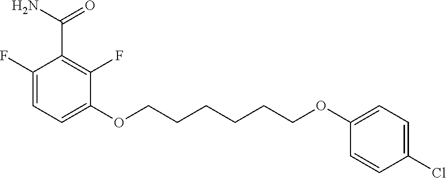 [SMILES: NC(=O)c1c(F)ccc(OCCCCCCOc2ccc(Cl)cc2)c1F] | 3-{[6-(4-chlorophenoxy)hexyl]oxy}-2,6- difluorobenzamide |
| 134 | 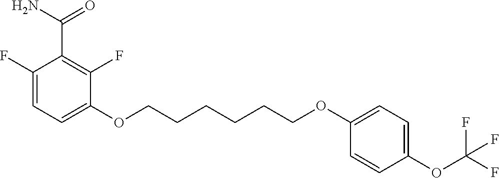 [SMILES: NC(=O)c1c(F)ccc(OCCCCCCOc2ccc(OC(F)(F)F)cc2)c1F] | 2,6-difluoro-3-({6-[4 (trifluoromethoxy)phenoxy]hexyl}oxy) benzamide |
| 135 | 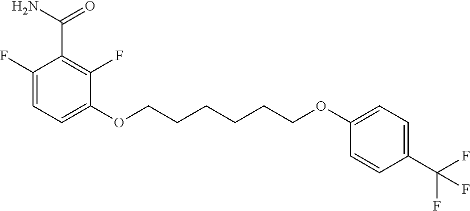 [SMILES: NC(=O)c1c(F)ccc(OCCCCCCOc2ccc(C(F)(F)F)cc2)c1F] | 2,6-difluoro-3-({6-[4- (trifluoromethyl)phenoxy]hexyl}oxy)benzamide |
| 136 | 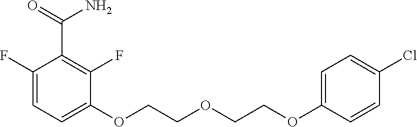 [SMILES: NC(=O)c1c(F)ccc(OCCOCCOc2ccc(Cl)cc2)c1F] | 3-{2-[2-(4-chlorophenoxy)ethoxy]ethoxy}-2,6- difluorobenzamide |
| 137 | 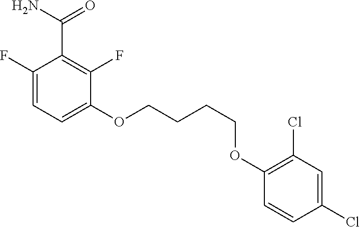 [SMILES: NC(=O)c1c(F)ccc(OCCCCOc2ccc(Cl)cc2Cl)c1F] | 3-[4-(2,4-dichlorophenoxy)butoxy]-2,6- difluorobenzamide |
| 138 | 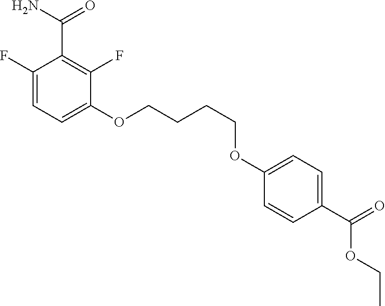 [SMILES: CCOC(=O)c1ccc(OCCCCOc2ccc(F)c(C(N)=O)c2F)cc1] | ethyl 4-[4-(3-carbamoyl-2,4- difluorophenoxy)butoxy]benzoate |
| 139 | 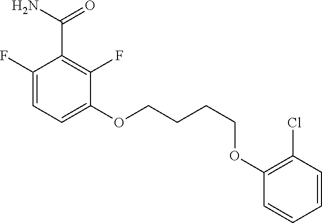 [SMILES: NC(=O)c1c(F)ccc(OCCCCOc2ccccc2Cl)c1F] | 3-[4-(2-chlorophenoxy)butoxy]- 2,6-difluorobenzamide |
| 140 | 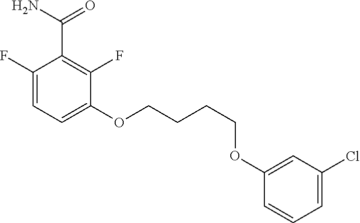 [SMILES: NC(=O)c1c(F)ccc(OCCCCOc2cccc(Cl)c2)c1F] | 3-[4-(3-chlorophenoxy)butoxy]- 2,6-difluorobenzamide |
| 141 | 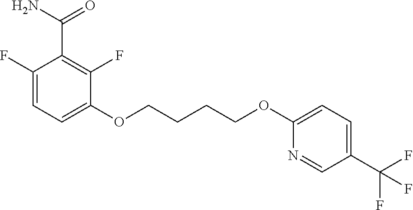 [SMILES: NC(=O)c1c(F)ccc(OCCCCOc2ccc(C(F)(F)F)cn2)c1F] | 2,6-difluoro-3-(4-{[5-(trifluoromethyl) pyridin-2-yl]oxy}butoxy)benzamide |
| 142 | 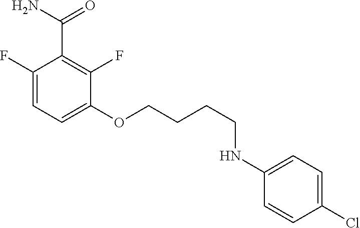 [SMILES: NC(=O)c1c(F)ccc(OCCCCNc2ccc(Cl)cc2)c1F] | 3-{4-[(4-chlorophenyl)amino]butoxy}-2,6- difluorobenzamide |
| 143 | 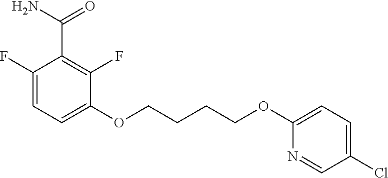 [SMILES: NC(=O)c1c(F)ccc(OCCCCOc2ccc(Cl)cn2)c1F] | 3-{4-[(5-chloropyridin-2-yl)oxy]butoxy}-2,6- difluorobenzamide |
| 144 | 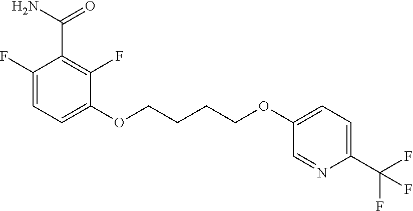 [SMILES: NC(=O)c1c(F)ccc(OCCCCOc2ccc(C(F)(F)F)nc2)c1F] | 2,6-difluoro-3-(4-{[6-(trifluoromethyl) pyridin-3-yl]oxy}butoxy)benzamide |
| 145 | 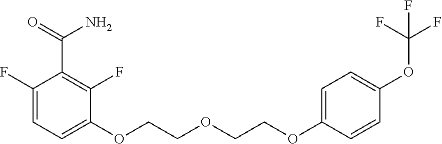 [SMILES: NC(=O)c1c(F)ccc(OCCOCCOc2ccc(OC(F)(F)F)cc2)c1F] | 2,6-difluoro-3-(2-{2-[4- (trifluoromethoxy)phenoxy]ethoxy}ethoxy) benzamide |
| 146 | 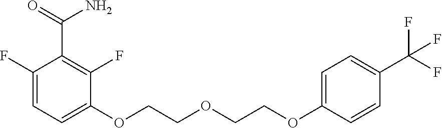 [SMILES: NC(=O)c1c(F)ccc(OCCOCCOc2ccc(C(F)(F)F)cc2)c1F] | 2,6-difluoro-3-(2-{2-[4- (trifluoromethyl)phenoxy]ethoxy}ethoxy) benzamide |
| 147 | 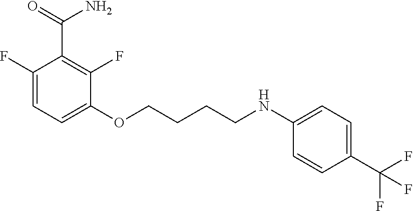 [SMILES: NC(=O)c1c(F)ccc(OCCCCNc2ccc(C(F)(F)F)cc2)c1F] | 2,6-difluoro-3-(4-{[4- (trifluoromethyl)phenyl]amino}butoxy) benzamide |
| 148 | 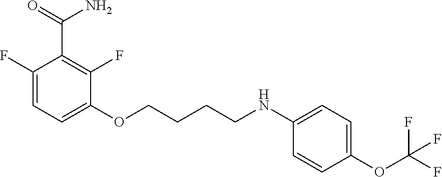 [SMILES: NC(=O)c1c(F)ccc(OCCCCNc2ccc(OC(F)(F)F)cc2)c1F] | 2,6-difluoro-3-(4-{[4- (trifluoromethoxy)phenyl]amino}butoxy) benzamide |
| 149 | 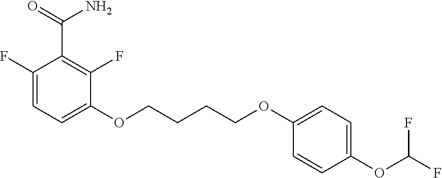 [SMILES: NC(=O)c1c(F)ccc(OCCCCOc2ccc(OC(F)F)cc2)c1F] | 3-{4-[4-(difluoromethoxy)phenoxylbutoxy}-2,6- difluorobenzamide |
| 150 | 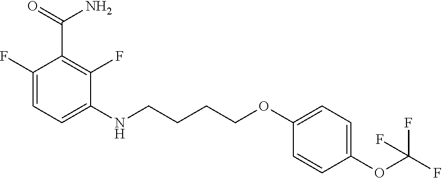 [SMILES: NC(=O)c1c(F)ccc(NCCCCOc2ccc(OC(F)(F)F)cc2)c1F] | 2,6-difluoro-3-({4-[4- (trifluoromethoxy)phenoxy]butyl}amino) benzamide |
| 151 | 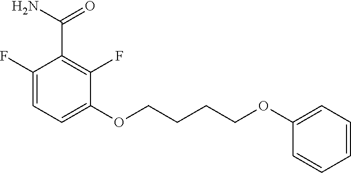 [SMILES: NC(=O)c1c(F)ccc(OCCCCOc2ccccc2)c1F] | 2,6-difluoro-3-(4-phenoxybutoxy)benzamide |
| 152 | 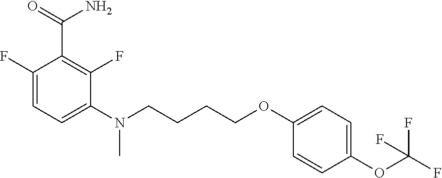 [SMILES: CN(CCCCOc1ccc(OC(F)(F)F)cc1)c1ccc(F)c(C(N)=O)c1F] | 2,6-difluoro-3-(methyl{4-[4- (trifluoromethoxy)phenoxy]butyl}amino) benzamide |
| 153 | 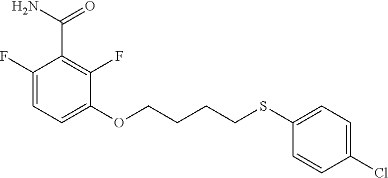 [SMILES: NC(=O)c1c(F)ccc(OCCCCSc2ccc(Cl)cc2)c1F] | 3-{4-[(4-chlorophenyl)thio]butoxy}-2,6- difluorobenzamide |
| 154 | 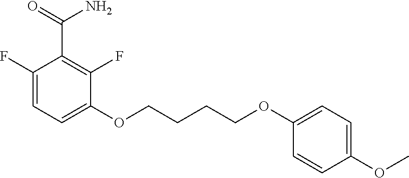 [SMILES: COc1ccc(OCCCCOc2ccc(F)c(C(N)=O)c2F)cc1] | 2,6-difluoro-3-[4-(4- methoxyphenoxy)butoxy]benzamide |
| 155 | 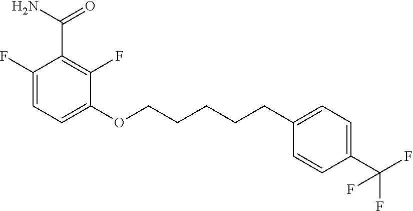 [SMILES: NC(=O)c1c(F)ccc(OCCCCCc2ccc(C(F)(F)F)cc2)c1F] | 2,6-difluoro-3-({5-[4- (trifluoromethyl)phenyl]pentyl}oxy)benzamide |
| 156 | 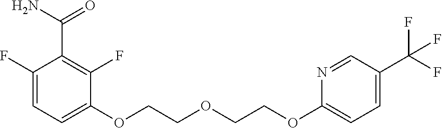 [SMILES: NC(=O)c1c(F)ccc(OCCOCCOc2ccc(C(F)(F)F)cn2)c1F] | 2,6-difluoro-3-[2-(2-{[5-(trifluoromethyl) pyridin-2-yl]oxy}ethoxy) ethoxy]benzamide |
| 157 | 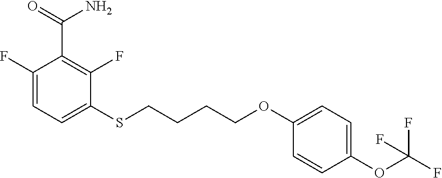 [SMILES: NC(=O)c1c(F)ccc(SCCCCOc2ccc(OC(F)(F)F)cc2)c1F] | 2,6-difluoro-3-({4-[4- (trifluoromethoxy)phenoxy]butyl}thio)benzamide |
| 158 | 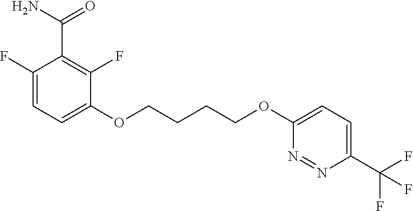 [SMILES: NC(=O)c1c(F)ccc(OCCCCOc2ccc(C(F)(F)F)nn2)c1F] | 2,6-difluoro-3-(4-{[6-(trifluoromethyl) pyridazin-3-yl]oxy}butoxy)benzamide |
| 159 | 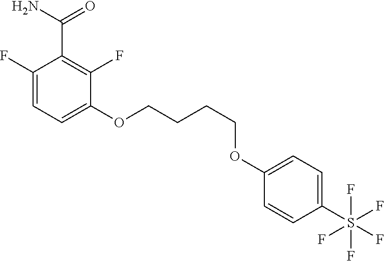 [SMILES: NC(=O)c1c(F)ccc(OCCCCOc2ccc(S(F)(F)(F)(F)F)cc2)c1F] | 2,6-difluoro-3-{4-4-[4-pentafluoro- lambda~6~-sulfanyl)phenoxy] butoxy}benzamide |
| 160 | 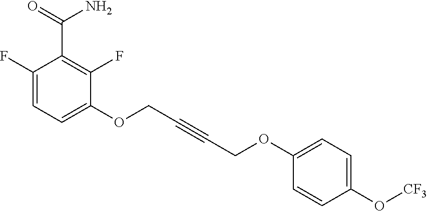 [SMILES: NC(=O)c1c(F)ccc(OCC#CCOc2ccc(OC(F)(F)F)cc2)c1F] | 2,6-difluoro-3-({4-[4-(trifluoromethoxy) phenoxy]but-2-yn-1-yl}oxy)benzamide |
| 161 | 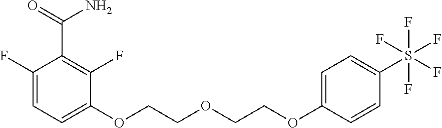 [SMILES: NC(=O)c1c(F)ccc(OCCOCCOc2ccc(S(F)(F)(F)(F)F)cc2)c1F] | 2,6-difluoro-3-(2-{2-[4-(pentafluoro- lambda~6~-sulfanyl)phenoxy] ethoxy}ethoxy)benzamide |
| 162 | 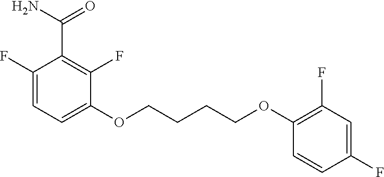 [SMILES: NC(=O)c1c(F)ccc(OCCCCOc2ccc(F)cc2F)c1F] | 3-[4-(2,4-difluorophenoxy)butoxy]-2,6- difluorobenzamide |
| 163 | 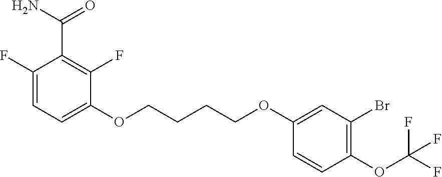 [SMILES: NC(=O)c1c(F)ccc(OCCCCOc2ccc(OC(F)(F)F)c(Br)c2)c1F] | 3-{4-[3-bromo-4-(trifluoromethoxy)phenoxy] butoxy}-2,6-difluorobenzamide |
| 164 | 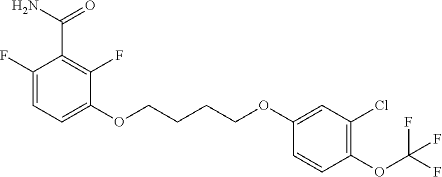 [SMILES: NC(=O)c1c(F)ccc(OCCCCOc2ccc(OC(F)(F)F)c(Cl)c2)c1F] | 3-{4-[3-chloro-4-(trifluoromethoxy)phenoxy] butoxy}-2,6-difluorobenzamide |
| 165 | 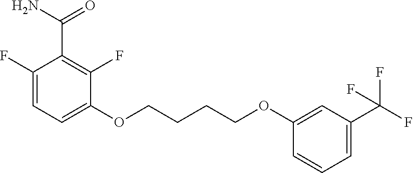 [SMILES: NC(=O)c1c(F)ccc(OCCCCOc2cccc(C(F)(F)F)c2)c1F] | 2,6-difluoro-3-{4-[3- (trifluoromethyl)phenoxy]butoxy}benzamide |
| 166 | 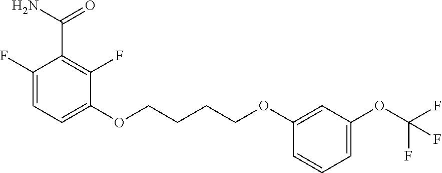 [SMILES: NC(=O)c1c(F)ccc(OCCCCOc2cccc(OC(F)(F)F)c2)c1F] | 2,6-difluoro-3-{4-[3- (trifluoromethoxy)phenoxy]butoxy} benzamide |
| 167 | 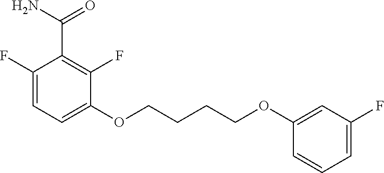 [SMILES: NC(=O)c1c(F)ccc(OCCCCOc2cccc(F)c2)c1F] | 2,6-difluoro-3-[4-(3-fluorophenoxy)butoxy] benzamide |
| 168 | 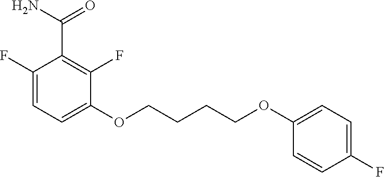 [SMILES: NC(=O)c1c(F)ccc(OCCCCOc2ccc(F)cc2)c1F] | 2,6-difluoro-3-[4-(4-fluorophenoxy)butoxy] benzamide |
| 169 | 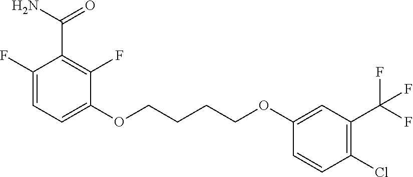 [SMILES: NC(=O)c1c(F)ccc(OCCCCOc2ccc(Cl)c(C(F)(F)F)c2)c1F] | 3-{4-[4-chloro-3-(trifluoromethyl)phenoxy] butoxy}-2,6-difluorobenzamide |
| 170 | 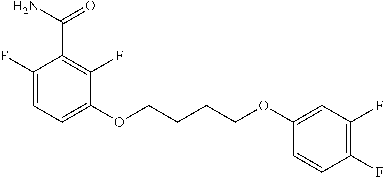 [SMILES: NC(=O)c1c(F)ccc(OCCCCOc2ccc(F)c(F)c2)c1F] | 3-[4-(3,4-difluorophenoxy)butoxy]-2,6- difluorobenzamide |
| 171 | 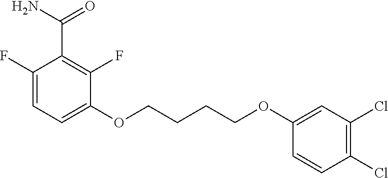 [SMILES: NC(=O)c1c(F)ccc(OCCCCOc2ccc(Cl)c(Cl)c2)c1F] | 3-[4-(3,4-dichlorophenoxy)butoxy]-2,6- difluorobenzamide |
| 172 | 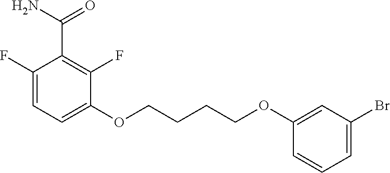 [SMILES: NC(=O)c1c(F)ccc(OCCCCOc2cccc(Br)c2)c1F] | 3-[4-(3-bromophenoxy)butoxy]-2,6- difluorobenzamide |
| 173 | 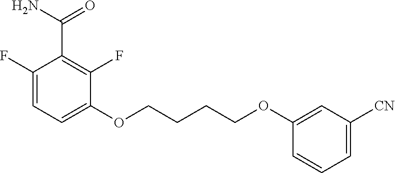 [SMILES: N#Cc1cccc(OCCCCOc2ccc(F)c(C(N)=O)c2F)c1] | 3-[4-(3-cyanophenoxy)butoxy]-2,6- difluorobenzamide |
| 174 | 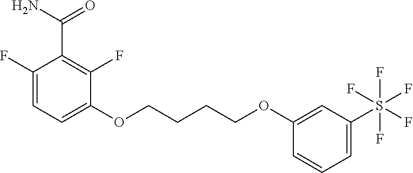 [SMILES: NC(=O)c1c(F)ccc(OCCCCOc2cccc(S(F)(F)(F)(F)F)c2)c1F] | 2,6-difluoro-3-{4-[3-(pentafluoro-lambda~6~- sulfanyl)phenoxy]butoxy}benzamide |
| 175 | 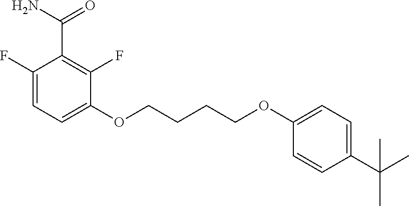 [SMILES: CC(C)(C)c1ccc(OCCCCOc2ccc(F)c(C(N)=O)c2F)cc1] | 3-[4-(4-tert-butylphenoxy)butoxy]-2,6- difluorobenzamide |
| 176 | 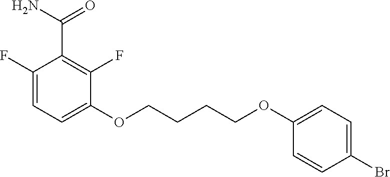 [SMILES: NC(=O)c1c(F)ccc(OCCCCOc2ccc(Br)cc2)c1F] | 3-[4-(4-bromophenoxy)butoxy]- 2,6-difluorobenzamide |
| 177 | 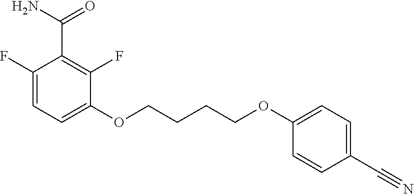 [SMILES: N#Cc1ccc(OCCCCOc2ccc(F)c(C(N)=O)c2F)cc1] | 3-[4-(4-cyanophenoxy)butoxy]-2,6- difluorobenzamide |
| 178 | 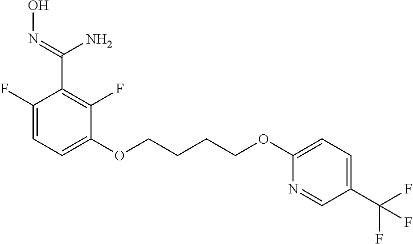 [SMILES: N/C(=N\O)c1c(F)ccc(OCCCCOc2ccc(C(F)(F)F)cn2)c1F] | 2,6-difluoro-N′-hydroxy-3-(4-{[5- (trifluoromethyl)pyridin-2- yl]oxy}butoxy)benzenecarboximidamide |
| 179 | 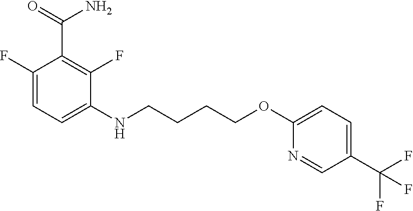 [SMILES: NC(=O)c1c(F)ccc(NCCCCOc2ccc(C(F)(F)F)cn2)c1F] | 2,6-difluoro-3-[(4-{[5-(trifluoromethyl) pyridin-2-yl]oxy}butyl)amino]benzamide |
| 180 | 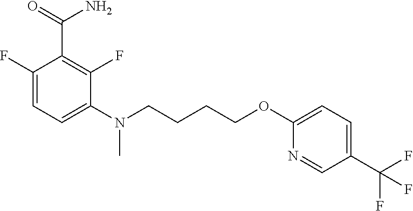 [SMILES: CN(CCCCOc1ccc(C(F)(F)F)cn1)c1ccc(F)c(C(N)=O)c1F] | 2,6-difluoro-3-[methyl(4-{[5-(trifluoromethyl) pyridin-2-yl]oxy}butyl) amino]benzamide |
| 181 | 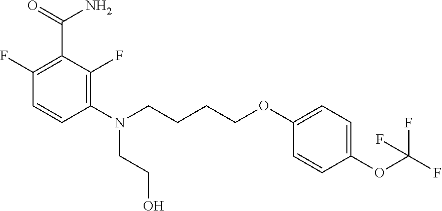 [SMILES: NC(=O)c1c(F)ccc(N(CCO)CCCCOc2ccc(OC(F)(F)F)cc2)c1F] | 2,6-difluoro-3-[(2-hydroxyethyl){4-[4- (trifluoromethoxy)phenoxy]butyl}amino] benzamide |
| 182 | 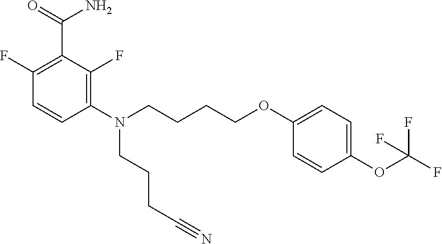 [SMILES: N#CCCCN(CCCCOc1ccc(OC(F)(F)F)cc1)c1ccc(F)c(C(N)=O)c1F] | 3-[(3-cyanopropyl){4-[4- (trifluoromethoxy)phenoxy]butyl)amino]-2,6- difluorobenzamide |
| 183 | 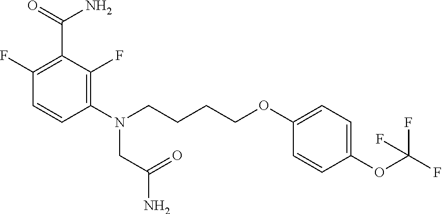 [SMILES: NC(=O)CN(CCCCOc1ccc(OC(F)(F)F)cc1)c1ccc(F)c(C(N)=O)c1F] | 3-[(2-amino-2-oxoethyl){4-[4- (trifluoromethoxy)phenoxy]butyl}amino]-2,6- difluorobenzamide |
| 184 | 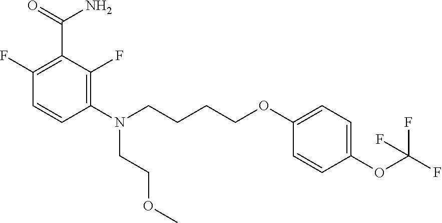 [SMILES: COCCN(CCCCOc1ccc(OC(F)(F)F)cc1)c1ccc(F)c(C(N)=O)c1F] | 2,6-difluoro-3-[(2-methoxyethyl){4-[4- (trifluoromethoxy)phenoxy]butyl} amino]benzamide |
| 185 | 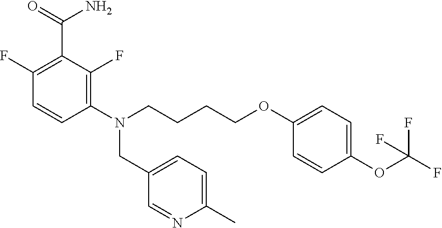 [SMILES: Cc1ccc(CN(CCCCOc2ccc(OC(F)(F)F)cc2)c2ccc(F)c(C(N)=O)c2F)cn1] | 2,6-difluoro-3-([(6-methylpyridin-3-yl) methyl]{4-[4-(trifluoromethoxy) phenoxy]butyl}amino)benzamide |
| 186 | 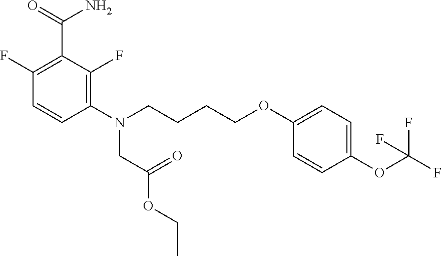 [SMILES: CCOC(=O)CN(CCCCOc1ccc(OC(F)(F)F)cc1)c1ccc(F)c(C(N)=O)c1F] | ethyl N-(3-carbamoyl-2,4-difluorophenyl)- N-{4-[4-(trifluoromethoxy)phenoxy] butyl}glycinate |
| 187 | 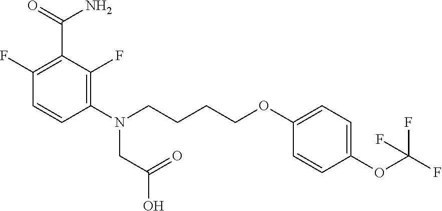 [SMILES: NC(=O)c1c(F)ccc(N(CCCCOc2ccc(OC(F)(F)F)cc2)CC(=O)O)c1F] | N-(3-carbamoyl-2,4-difluorophenyl)-N-{4-[4- (trifluoromethoxy)phenoxy]butyl}glycine |

| | | LCMS | |
|---|---|---|---|
| | | m/z = | |
| No. | Name | [M + H]⁺ | ¹H NMR |
| 124 | 3-[3-(4- | 342.1 | 1H NMR (400 MHz, CDCl3) δ 7.23 (d, J = 9.0 Hz, |
| | chlorophenoxy)propoxy]-2,6- | | 2H), 7.02 (td, J = 9.1, 5.1 Hz, |
| | difluorobenzamide | | 1H), 6.90-6.83 (m, 1H), 6.83 (d, J = 9.0 Hz, |
| | | | 2H), 5.94 (br s, 2H), 4.20 (t, J = 6.0 Hz, |
| | | | 2H), 4.14 (t, J = 6.0 Hz, 2H), 2.27 (p, |
| | | | J = 6.0 Hz, 2H). |
| 125 | 2,6-difluoro-3-{3-[4- | 375.9 | 1H NMR (400 MHz, Acetone) δ |
| | (trifluoromethyl)phenoxy]propoxy}benzamide | | 7.64 (dd, J = 9.0, 0.6 Hz, 2H), 7.39 (br s, 1H), |
| | | | 7.23 (td, J = 9.2, 5.2 Hz, 1H), 7.15 (d, J = 8.5 Hz, |
| | | | 2H), 7.14-7.07 (m, 1H), |
| | | | 7.00-6.91 (m, 1H), 4.31 (q, J = 6.1 Hz, 4H), |
| | | | 2.31 (p, J = 6.2 Hz, 2H). |
| 126 | 2,6-difluoro-3-{3-[4- | 392.1 | 1H NMR (400 MHz, CDCl3) δ |
| | (trifluoromethoxy)phenoxy]propoxy}benzamide | | 7.16-7.10 (m, 2H), 7.02 (td, J = 9.1, 5.1 Hz, |
| | | | 1H), 6.92-6.83 (m, 3H), 5.95 (br s, 1H), |
| | | | 5.91 (br s, 1H), 4.18 (dt, J = 19.4, 6.0 Hz, |
| | | | 4H), 2.32-2.22 (m, 2H). |
| 127 | 3-[4-(4- | 356.1 | 1H NMR (400 MHz, CDCl3) δ 7.22 (d, J = 9.1 Hz, |
| | chlorophenoxy)butoxy]-2,6- | | 2H), 7.00 (td, J = 9.1, 5.1 Hz, |
| | difluorobenzamide | | 1H), 6.87 (td, J = 9.1, 2.0 Hz, 1H), |
| | | | 6.81 (d, J = 9.0 Hz, 2H), 5.95 (br s, 1H), |
| | | | 5.90 (br s, 1H), 4.05 (dt, J = 31.1, 5.9 Hz, |
| | | | 4H), 2.04-1.93 (m, 4H). |
| 128 | 2,6-difluoro-3-{4-[4- | 406.2 | ¹H NMR (400 MHz, DMSO) δ 8.11 (br |
| | (trifluoromethoxy)phenoxy]butoxy}benzamide | | s, 1H), 7.84 (br s, 1H), 7.28 (dd, J = 9.1, |
| | | | 0.7 Hz, 2H), 7.22 (td, J = 9.4, 5.3 Hz, |
| | | | 1H), 7.07 (dd, J = 8.9, 1.8 Hz, 1H), |
| | | | 7.02 (d, J = 9.2 Hz, 2H), 4.14-4.02 (m, 4H), |
| | | | 1.92-1.81 (m, 4H). |
| 129 | 2,6-difluoro-3-{4-[4- | 390.0 | 1H NMR (400 MHz, CDCl3) δ 7.53 (d, J = 8.5 Hz, |
| | (trifluoromethyl)phenoxy]butoxy}benzamide | | 2H), 7.05-6.96 (m, 1H), |
| | | | 6.94 (d, J = 8.5 Hz, 2H), 6.87 (td, J = 9.1, 2.0 Hz, |
| | | | 1H), 6.14 (br s, 1H), 6.00 (br s, 1H), |
| | | | 4.13-4.04 (m, 4H), 2.05-1.97 (m, 4H). |
| 130 | 3-{[5-(4- | 370.1 | 1H NMR (400 MHz, CDCl3) δ |
| | chlorophenoxy)pentyl]oxy}- | | 7.24-7.19 (m, 2H), 6.99 (td, J = 9.1, 5.1 Hz, |
| | 2,6-difluorobenzamide | | 1H), 6.87 (td, J = 9.1, 2.0 Hz, 1H), |
| | | | 6.84-6.79 (m, 2H), 5.96 (br s, 1H), 5.88 (br s, |
| | | | 1H), 4.04 (t, J = 6.3 Hz, 2H), 3.96 (t, J = 6.3 Hz, |
| | | | 2H), 1.92-1.81 (m, 4H), |
| | | | 1.70-1.61 (m, 2H). |
| 131 | 2,6-difluoro-3-({5-[4- | 420.2 | 1H NMR (400 MHz, CDCl3) δ |
| | (trifluoromethoxy)phenoxy]pentyl}oxy)benzamide | | 7.16-7.10 (m, 2H), 7.00 (td, J = 9.1, 5.1 Hz, |
| | | | 1H), 6.90-6.84 (m, 3H), 5.96 (br s, 1H), |
| | | | 5.90 (br s, 1H), 4.04 (t, J = 6.3 Hz, 2H), |
| | | | 3.97 (t, J = 6.3 Hz, 2H), 1.93-1.82 (m, |
| | | | 4H), 1.71-1.61 (m, 2H). |
| 132 | 2,6-difluoro-3-({5-[4- | 403.8 | 1H NMR (400 MHz, CDCl3) δ 7.53 (d, J = 8.5 Hz, |
| | (trifluoromethyl)phenoxy]pentyl}oxy)benzamide | | 2H), 7.00 (td, J = 9.1, 5.2 Hz, |
| | | | 1H), 6.95 (d, J = 8.5 Hz, 2H), 6.87 (td, J = 9.1, |
| | | | 2.0 Hz, 1H), 5.96 (br s, 1H), |
| | | | 5.88 (br s, 1H), 4.04 (q, J = 6.2 Hz, 4H), |
| | | | 1.94-1.84 (m, 4H), 1.72-1.62 (m, 2H). |
| 133 | 3-{[6-(4- | 384.1 | 1H NMR (400 MHz, CDCl3) δ |
| | chlorophenoxy)hexyl]oxy}- | | 7.24-7.19 (m, 2H), 6.99 (td, J = 9.1, 5.2 Hz, |
| | 2,6-difluorobenzamide | | 1H), 6.87 (td, J = 9.1, 2.0 Hz, 1H), |
| | | | 6.84-6.78 (m, 2H), 5.96 (br s, 1H), 5.90 (br s, |
| | | | 1H), 4.02 (t, J = 6.4 Hz, 2H), 3.93 (t, J = 6.4 Hz, |
| | | | 2H), 1.88-1.76 (m, 4H), |
| | | | 1.56-1.50 (m, 4H). |
| 134 | 2,6-difluoro-3-({6-[4- | 434.2 | 1H NMR (400 MHz, CDCl3) δ |
| | (trifluoromethoxy)phenoxy]hexyl}oxy)benzamide | | 7.24-7.19 (m, 2H), 6.99 (td, J = 9.1, 5.2 Hz, |
| | | | 1H), 6.87 (td, J = 9.1, 2.0 Hz, 1H), |
| | | | 6.84-6.78 (m, 2H), 5.96 (br s, 1H), 5.90 (br s, |
| | | | 1H), 4.02 (t, J = 6.4 Hz, 2H), 3.93 (t, J = 6.4 Hz, |
| | | | 2H), 1.88-1.76 (m, 4H), |
| | | | 1.56-1.50 (m, 4H). |
| 135 | 2,6-difluoro-3-({6-[4- | 417.9 | 1H NMR (400 MHz, CDCl3) δ 7.53 (d, J = 8.5 Hz, |
| | (trifluoromethyl)phenoxy]hexyl}oxy)benzamide | | 2H), 6.99 (td, J = 9.1, 5.2 Hz, |
| | | | 1H), 6.94 (d, J = 8.5 Hz, 2H), 6.87 (td, J = 9.1, |
| | | | 2.0 Hz, 1H), 5.95 (br s, 1H), |
| | | | 5.88 (br s, 1H), 4.02 (q, J = 6.3 Hz, 4H), |
| | | | 1.84 (p, J = 6.6 Hz, 4H), 1.58-1.52 (m, 4H). |
| 136 | 3-{2-[2-(4- | 372.1 | ¹H NMR (400 MHz, DMSO) δ 8.10 (s, |
| | chlorophenoxy)ethoxy]ethoxy}- | | 1H), 7.82 (s, 1H), 7.31 (d, J = 9.1 Hz, |
| | 2,6-difluorobenzamide | | 2H), 7.23 (td, J = 9.4, 5.3 Hz, 1H), |
| | | | 7.04 (td, J = 9.0, 1.9 Hz, 1H), 6.97 (d, J = 9.1 Hz, |
| | | | 2H), 4.21-4.17 (m, 2H), |
| | | | 4.12-4.08 (m, 2H), 3.83-3.78 (m, 4H). |
| 137 | 3-[4-(2,4- | 390.1 | ¹H NMR (400 MHz, Acetone) δ 7.44 (d, |
| | dichlorophenoxy)butoxy]-2,6- | | J = 2.6 Hz, 1H), 7.38 (br s, 1H), 7.31 (dd, |
| | difluorobenzamide | | J = 8.8, 2.6 Hz, 1H), 7.19 (td, J = 9.3, 5.2 Hz, |
| | | | 1H), 7.15 (d, J = 8.9 Hz, 1H), |
| | | | 7.10 (br s, 1H), 6.95 (ddd, J = 9.2, 8.7, 2.1 Hz, |
| | | | 1H), 4.24-4.17 (m, 4H), 2.04-2.01 (m, |
| | | | 4H). |
| 138 | ethyl 4-[4-(3-carbamoyl-2,4- | 394.0 | ¹H NMR (400 MHz, Acetone) δ 7.96 (d, |
| | difluorophenoxy)butoxy]benzoate | | J = 9.0 Hz, 2H), 7.39 (br s, 1H), 7.20 (td, |
| | | | J = 9.2, 5.2 Hz, 1H), 7.11 (br s, 1H), |
| | | | 7.03 (d, J = 8.9 Hz, 2H), 6.96 (td, J = 9.0, 2.0 Hz, |
| | | | 1H), 4.30 (q, J = 7.1 Hz, 2H), |
| | | | 4.22-4.15 (m, 4H), 2.03-1.98 (m, 4H), |
| | | | 1.34 (t, J = 7.1 Hz, 3H). |
| 139 | 3-[4-(2- | 356.1 | ¹H NMR (400 MHz, DMSO) δ 8.09 (br |
| | chlorophenoxy)butoxy]-2,6- | | s, 1H), 7.81 (br s, 1H), 7.41 (dd, J = 7.9, |
| | difluorobenzamide | | 1.6 Hz, 1H), 7.29 (ddd, J = 8.3, 7.4, 1.6 Hz, |
| | | | 1H), 7.22 (td, J = 9.3, 5.3 Hz, 1H), |
| | | | 7.14 (dd, J = 8.3, 1.4 Hz, 1H), 7.05 (td, J = 9.0, |
| | | | 1.9 Hz, 1H), 6.97-6.92 (m, 1H), |
| | | | 4.16-4.10 (m, 4H), 1.93-1.89 (m, 4H). |
| 140 | 3-[4-(3- | 356.1 | ¹H NMR (400 MHz, CDCl₃) δ 7.19 (t, J = 8.1 Hz, |
| | chlorophenoxy)butoxy]-2,6- | | 1H), 7.00 (td, J = 9.1, 5.1 Hz, |
| | difluorobenzamide | | 1H), 6.94-6.85 (m, 3H), 6.77 (ddd, J = 8.3, |
| | | | 2.4, 0.7 Hz, 1H), 5.95 (br s, 2H), |
| | | | 4.11-4.07 (m, 2H), 4.05-4.00 (m, 2H), |
| | | | 2.04-1.94 (m, 4H). |
| 141 | 2,6-difluoro-3-(4-{[5- | 391.1 | ¹H NMR (400 MHz, DMSO-d₆) δ |
| | (trifluoromethyl)pyridin-2- | | 8.59-8.56 (m, 1H), 8.11 (br s, 1H), 8.06 (ddd, |
| | yl]oxy}butoxy)benzamide | | J = 8.8, 2.6, 0.5 Hz, 1H), 7.83 (br s, 1H), |
| | | | 7.22 (td, J = 9.4, 5.3 Hz, 1H), 7.05 (td, J = 9.0, |
| | | | 1.9 Hz, 1H), 7.01 (d, J = 8.8 Hz, |
| | | | 1H), 4.45-4.32 (m, 2H), 4.16-4.03 (m, |
| | | | 2H), 1.96-1.78 (m, 4H). |
| 142 | 3-{4-[(4- | 355.2 | ¹H NMR (400 MHz, Acetone) δ 7.39 (br |
| | chlorophenyl)amino]butoxy}- | | s, 1H), 7.18 (td, J = 9.3, 5.2 Hz, 1H), |
| | 2,6-difluorobenzamide | | 7.11 (br s, 1H), 7.07 (d, J = 8.9 Hz, 2H), |
| | | | 6.95 (td, J = 9.0, 2.0 Hz, 1H), 6.63 (d, J = 8.9 Hz, |
| | | | 2H), 5.15-5.07 (m, 1H), |
| | | | 4.13 (t, J = 6.3 Hz, 2H), 3.22-3.16 (m, 2H), |
| | | | 1.97-1.86 (m, 2H), 1.87-1.76 (m, 2H). |
| 143 | 3-{4-[(5-chloropyridin-2- | 357.1 | ¹H NMR (400 MHz, Acetone) δ 8.12 (d, |
| | yl)oxy]butoxy}-2,6- | | J = 2.7 Hz, 1H), 7.70 (dd, J = 8.8, 2.7 Hz, |
| | difluorobenzamide | | 1H), 7.41 (br s, 1H), 7.19 (td, J = 9.2, |
| | | | 5.2 Hz, 1H), 7.13 (br s, 1H), |
| | | | 6.95 (td, J = 9.0, 2.0 Hz, 1H), 6.81 (d, J = 8.8 Hz, |
| | | | 1H), 4.39-4.33 (m, 2H), |
| | | | 4.19-4.13 (m, 2H), 1.98-1.93 (m, 4H). |
| 144 | 2,6-difluoro-3-(4-{[6- | 391.2 | ¹H NMR (400 MHz, DMSO) δ 8.44 (d, J = 2.8 Hz, |
| | (trifluoromethyl)pyridin-3- | | 1H), 8.10 (br s, 1H), 7.84 (d, J = 8.8 Hz, |
| | yl]oxy}butoxy)benzamide | | 1H), 7.82 (br s, 1H), 7.60 (dd, J = 8.6, |
| | | | 2.7 Hz, 1H), 7.23 (td, J = 9.3, 5.3 Hz, |
| | | | 1H), 7.06 (td, J = 9.0, 1.9 Hz, 1H), |
| | | | 4.22 (t, J = 6.0 Hz, 2H), 4.12 (t, J = 5.9 Hz, |
| | | | 2H), 1.97-1.84 (m, 4H). |
| 145 | 2,6-difluoro-3-(2-{2-[4- | 422.2 | ¹H NMR (400 MHz, Acetone) δ 7.42 (br |
| | (trifluoromethoxy)phenoxy]ethoxy}ethoxy)benzamide | | s, 1H), 7.29-7.17 (m, 3H), 7.14 (br s, |
| | | | 1H), 7.05 (d, J = 9.2 Hz, 2H), 6.94 (td, J = 9.0, |
| | | | 2.0 Hz, 1H), 4.26-4.22 (m, 2H), |
| | | | 4.21-4.16 (m, 2H), 3.93-3.88 (m, 4H). |
| 146 | 2,6-difluoro-3-(2-{2-[4- | 406.0 | ¹H NMR (400 MHz, DMSO) δ 8.09 (br |
| | (trifluoromethyl)phenoxy]ethoxy}ethoxy)benzamide | | s, 1H), 7.82 (br s, 1H), 7.64 (d, J = 8.5 Hz, |
| | | | 2H), 7.23 (td, J = 9.4, 5.3 Hz, 1H), |
| | | | 7.13 (d, J = 8.5 Hz, 2H), 7.04 (td, J = 9.0, |
| | | | 1.9 Hz, 1H), 4.23-4.17 (m, 4H), |
| | | | 3.86-3.80 (m, 4H). |
| 147 | 2,6-difluoro-3-(4-{[4- | 389.1 | ¹H NMR (400 MHz, DMSO) δ 8.09 (s, |
| | (trifluoromethyl)phenyl]amino}butoxy)benzamide | | 1H), 7.81 (s, 1H), 7.35 (d, J = 8.6 Hz, |
| | | | 2H), 7.22 (td, J = 9.3, 5.3 Hz, 1H), |
| | | | 7.05 (td, J = 9.0, 1.9 Hz, 1H), 6.66 (d, J = 8.6 Hz, |
| | | | 2H), 6.38 (t, J = 5.4 Hz, 1H), 4.08 (t, |
| | | | J = 6.4 Hz, 2H), 3.12 (dd, J = 12.6, 6.8 Hz, |
| | | | 2H), 1.87-1.77 (m, 2H), |
| | | | 1.74-1.64 (m, 2H). |
| 148 | 2,6-difluoro-3-(4-{[4- | 405.2 | ¹H NMR (400 MHz, DMSO) δ 8.09 (s, |
| | (trifluoromethoxy)phenyl]amino}butoxy)benzamide | | 1H), 7.81 (s, 1H), 7.21 (td, J = 9.3, 5.3 Hz, |
| | | | 1H), 7.08-7.01 (m, 3H), |
| | | | 6.67-6.49 (m, 2H), 5.88 (t, J = 5.5 Hz, 1H), 4.08 (t, |
| | | | J = 6.4 Hz, 2H), 3.05 (dd, J = 12.7, 6.8 Hz, |
| | | | 2H), 1.86-1.77 (m, 2H), |
| | | | 1.72-1.63 (m, 2H). |
| 149 | 3-{4-[4- | 388.0 | ¹H NMR (400 MHz, DMSO) δ 8.09 (br |
| | (difluoromethoxy)phenoxy]butoxy}- | | s, 1H), 7.81 (br s, 1H), 7.22 (td, J = 9.3, |
| | 2,6-difluorobenzamide | | 5.3 Hz, 1H), 7.10 (d, J = 9.1 Hz, 2H), |
| | | | 7.07 (t, J = 74.6 Hz, 1H), 7.08-7.02 (m, |
| | | | 1H), 6.96 (d, J = 9.1 Hz, 2H), 4.11 (t, J = 5.8 Hz, |
| | | | 2H), 4.02 (t, J = 5.9 Hz, 2H), |
| | | | 1.93-1.81 (m, 4H). |
| 150 | 2,6-difluoro-3-({4-[4- | 405.0 | 1H NMR (400 MHz, Acetone-d6) δ |
| | (trifluoromethoxy)phenoxy]butyl}amino)benzamide | | 7.36-7.19 (m, 3H), 7.08-6.93 (m, 3H), |
| | | | 6.88-6.74 (m, 2H), 4.81 (brs, 1H), 4.09 (t, J = 6.2 Hz, |
| | | | 2H), 3.33-3.21 (m, 2H), |
| | | | 1.96-1.80 (m, 4H). |
| 151 | 2,6-difluoro-3-(4- | 322.0 | 1H NMR (400 MHz, Acetone-d6) δ |
| | phenoxybutoxy)benzamide | | 7.39 (brs, 1H), 7.27 (dd, J = 8.8, 7.3 Hz, 2H), |
| | | | 7.20 (td, J = 9.2, 5.2 Hz, 1H), 7.11 (brs, |
| | | | 1H), 7.00-6.86 (m, 4H), 4.17 (t, J = 5.9 Hz, |
| | | | 2H), 4.08 (t, J = 6.0 Hz, 2H), |
| | | | 2.04-1.91 (m, 4H). |
| 152 | 2,6-difluoro-3-(methyl{4-[4- | 419.1 | ¹H NMR (400 MHz, DMSO-d₆) δ |
| | (trifluoromethoxy)phenoxy]butyl}amino)benzamide | | 8.06 (br s, 1H), 7.77 (br s, IH), 7.27 (d, J = 9.1 Hz, |
| | | | 2H), 7.11-6.94 (m, 4H), 3.99 (t, |
| | | | J = 6.3 Hz, 2H), 3.07 (t, J = 7.2 Hz, 2H), |
| | | | 2.73 (s, 3H), 1.77-1.66 (m, 2H), |
| | | | 1.68-1.56 (m, 2H). |
| 153 | 3-{4-[(4- | 371.9 | 1H NMR (400 MHz, Acetone-d6) δ |
| | chlorophenyl)thio]butoxy}- | | 7.45-7.36 (m, 3H), 7.33 (d, J = 8.9 Hz, 2H), |
| | 2,6-difluorobenzamide | | 7.17 (td, J = 9.2, 5.2 Hz, 1H), 7.11 (brs, |
| | | | 1H), 6.95 (td, J = 8.9, 2.0 Hz, 1H), |
| | | | 4.12 (t, J = 6.2 Hz, 2H), 3.13-3.01 (m, 2H), |
| | | | 2.01-1.90 (m, 2H), 1.90-1.78 (m, 2H). |
| 154 | 2,6-difluoro-3-[4-(4- | 352.0 | ¹H NMR (400 MHz, DMSO) δ 8.09 (br |
| | methoxyphenoxy)butoxy]benzamide | | s, 1H), 7.81 (br s, 1H), 7.22 (td, J = 9.3, |
| | | | 5.3 Hz, 1H), 7.05 (td, J = 9.0, 1.9 Hz, |
| | | | 1H), 6.88-6.82 (m, 4H), 4.10 (t, J = 6.0 Hz, |
| | | | 2H), 3.96 (t, J = 6.1 Hz, 2H), 3.69 (s, |
| | | | 3H), 1.89-1.80 (m, 4H). |
| 155 | 2,6-difluoro-3-({5-[4- | 387.9 | ¹H NMR (400 MHz, CDCl₃) δ 7.53 (d, J = 8.0 Hz, |
| | (trifluoromethyl)phenyl]pentyl}oxy)benzamide | | 2H), 7.29 (d, J = 8.0 Hz, 2H), |
| | | | 6.97 (td, J = 9.1, 5.2 Hz, 1H), 6.86 (td, J = 9.1, |
| | | | 2.0 Hz, 1H), 5.94 (br s, 2H), |
| | | | 4.00 (t, J = 6.4 Hz, 2H), 2.70 (t, J = 7.7 Hz, |
| | | | 2H), 1.87-1.79 (m, 2H), 1.75-1.66 (m, |
| | | | 2H), 1.56-1.47 (m, 2H). |
| 156 | 2,6-difluoro-3-[2-(2-{[5- | 407.0 | ¹H NMR (400 MHz, DMSO) δ |
| | (trifluoromethyl)pyridin-2- | | 8.59-8.56 (m, 1H), 8.09 (br s, 1H), 8.06 (ddd, |
| | yl]oxy}ethoxy)ethoxy]benzamide | | J = 8.8, 2.6, 0.4 Hz, 1H), 7.81 (br s, 1H), |
| | | | 7.22 (td, J = 9.3, 5.3 Hz, 1H), |
| | | | 7.06-7.00 (m, 2H), 4.51-4.45 (m, 2H), |
| | | | 4.21-4.15 (m, 2H), 3.86-3.78 (m, 4H). |
| 157 | 2,6-difluoro-3-({4-[4- | 421.9 | 1H NMR (400 MHz, CDCl3) δ |
| | (trifluoromethoxy)phenoxy]butyl}thio)benzamide | | 7.47 (ddd, J = 8.8, 8.1, 6.1 Hz, 1H), |
| | | | 7.17-7.07 (m, 2H), 6.93 (td, J = 8.9, 1.5 Hz, |
| | | | 1H), 6.85 (d, J = 9.2 Hz, 2H), 5.95 (s, |
| | | | 2H), 3.95 (t, J = 6.1 Hz, 2H), 2.95 (t, J = 7.2 Hz, |
| | | | 2H), 1.98-1.86 (m, 2H), |
| | | | 1.84-1.71 (m, 2H). |
| 158 | 2,6-difluoro-3-(4-{[6- | 392.0 | ¹H NMR (400 MHz, DMSO) δ 8.13 (d, J = 9.3 Hz, |
| | (trifluoromethyl)pyridazin-3- | | 1H), 8.09 (br s, 1H), 7.81 (br s, |
| | yl]oxy}butoxy)benzamide | | 1H), 7.48 (dd, J = 9.3, 0.5 Hz, 1H), |
| | | | 7.23 (td, J = 9.3, 5.3 Hz, 1H), 7.05 (td, J = 9.0, |
| | | | 1.9 Hz, 1H), 4.61 (t, J = 6.2 Hz, 2H), |
| | | | 4.12 (t, J = 6.1 Hz, 2H), 2.01-1.86 (m, |
| | | | 4H). |
| 159 | 2,6-difluoro-3-{4-[4- | 447.8 | ¹H NMR (400 MHz, CDCl₃) δ 7.67 (d, J = 9.3 Hz, |
| | (pentafluoro-lambda~6~- | | 2H), 7.00 (td, J = 9.1, 5.1 Hz, |
| | sulfanyl)phenoxy]butoxy}benzamide | | 1H), 6.93-6.84 (m, 3H), 5.95 (br s, 2H), |
| | | | 4.13-4.04 (m, 4H), 2.07-1.94 (m, 4H). |
| 160 | 2,6-difluoro-3-({4-[4- | 402.0 | 1H NMR (400 MHz, CDCl3) δ 7.18 (d, J = 8.5 Hz, |
| | (trifluoromethoxy)phenoxy]but- | | 2H), 7.12-7.03 (m, 1H), |
| | 2-yn-1-yl}oxy)benzamide | | 6.99-6.89 (m, 2H), 6.89-6.79 (m, 1H), |
| | | | 6.41 (s, 1H), 6.10 (s, 1H), 4.82 (t, J = 1.6 Hz, |
| | | | 2H), 4.75 (t, J = 1.6 Hz, 2H). |
| 161 | 2,6-difluoro-3-(2-{2-[4- | 463.8 | ¹H NMR (400 MHz, DMSO) δ 8.09 (br |
| | (pentafluoro-lambda~6~- | | s, 1H), 7.84-7.78 (m, 3H), 7.22 (td, J = 9.4, |
| | sulfanyl)phenoxy]ethoxy}ethoxy)benzamide | | 5.3 Hz, 1H), 7.11 (d, J = 9.2 Hz, |
| | | | 2H), 7.04 (td, J = 9.0, 1.9 Hz, 1H), |
| | | | 4.25-4.16 (m, 4H), 3.86-3.80 (m, 4H). |
| 162 | 3-[4-(2,4- | 358.1 | ¹H NMR (400 MHz, Acetone) δ 7.39 (br |
| | difluorophenoxy)butoxy]-2,6- | | s, 1H), 7.18 (qd, J = 9.4, 5.3 Hz, 2H), |
| | difluorobenzamide | | 7.11 (br s, 1H), 7.04 (ddd, J = 11.5, 8.7, |
| | | | 3.0 Hz, 1H), 6.98-6.92 (m, 1H), |
| | | | 6.95-6.88 (m, 1H), 4.21-4.13 (m, 4H), |
| | | | 2.03-1.96 (m, 4H). |
| 163 | 3-{4-[3-bromo-4- | 484.2 | ¹H NMR (400 MHz, Acetone) δ |
| | (trifluoromethoxy)phenoxy]butoxy}- | | 7.40 (ddd, J = 9.1, 2.6, 1.3 Hz, 1H), 7.38 (br s, |
| | 2,6-difluorobenzamide | | 1H), 7.32 (d, J = 3.0 Hz, 1H), 7.20 (td, J = 9.2, |
| | | | 5.2 Hz, 1H), 7.11 (br s, 1H), |
| | | | 7.07 (dd, J = 9.1, 3.0 Hz, 1H), 6.96 (ddd, J = 9.2, |
| | | | 8.7, 2.1 Hz, 1H), 4.20-4.15 (m, |
| | | | 4H), 2.02-1.97 (m, 4H). |
| 164 | 3-{4-[3-chloro-4- | 440.2 | ¹H NMR (400 MHz, Acetone) δ |
| | (trifluoromethoxy)phenoxy]butoxy}- | | 7.41 (dd, J = 9.1, 1.2 Hz, 1H), 7.39 (br s, 1H), |
| | 2,6-difluorobenzamide | | 7.20 (dt, J = 9.3, 5.0 Hz, 1H), 7.18 (d, J = 3.0 Hz, |
| | | | 1H), 7.12 (br s, 1H), 7.02 (dd, J = 9.1, |
| | | | 3.0 Hz, 1H), 6.96 (td, J = 8.9, 2.0 Hz, |
| | | | 1H), 4.21-4.14 (m, 4H), |
| | | | 2.03-1.97 (m, 4H). |
| 165 | 2,6-difluoro-3-{4-[3- | 390.0 | ¹H NMR (400 MHz, Acetone) δ 7.51 (t, J = 7.9 Hz, |
| | (trifluoromethyl)phenoxy]butoxy}benzamide | | 1H), 7.39 (br s, 1H), |
| | | | 7.28-7.22 (m, 3H), 7.20 (td, J = 9.2, 5.3 Hz, |
| | | | 1H), 7.11 (br s, 1H), 6.95 (td, J = 9.0, 2.0 Hz, |
| | | | 1H), 4.22-4.16 (m, 4H), |
| | | | 2.03-1.99 (m, 4H). |
| 166 | 2,6-difluoro-3-{4-[3- | 406.1 | ¹H NMR (400 MHz, Acetone) δ |
| | (trifluoromethoxy)phenoxy]butoxy}benzamide | | 7.43-7.37 (m, 2H), 7.20 (td, J = 9.2, 5.2 Hz, |
| | | | 1H), 7.11 (br s, 1H), 7.01-6.96 (m, 1H), |
| | | | 6.95 (td, J = 9.0, 2.5 Hz, 1H), |
| | | | 6.91-6.87 (m, 2H), 4.21-4.13 (m, 4H), |
| | | | 2.03-1.97 (m, 4H). |
| 167 | 2,6-difluoro-3-[4-(3- | 340.1 | ¹H NMR (400 MHz, Acetone) δ 7.39 (br |
| | fluorophenoxy)butoxy]benzamide | | s, 1H), 7.29 (td, J = 8.3, 7.1 Hz, 1H), |
| | | | 7.20 (td, J = 9.2, 5.2 Hz, 1H), 7.11 (br s, |
| | | | 1H), 6.96 (td, J = 9.0, 2.0 Hz, 1H), |
| | | | 6.78 (dd, J = 8.3, 2.3 Hz, 1H), 6.75-6.65 (m, |
| | | | 2H), 4.20-4.15 (m, 2H), 4.14-4.09 (m, |
| | | | 2H), 2.02-1.96 (m, 4H). |
| 168 | 2,6-difluoro-3-[4-(4- | 340.0 | ¹H NMR (400 MHz, Acetone) δ 7.39 (br |
| | fluorophenoxy)butoxy]benzamide | | s, 1H), 7.19 (td, J = 9.2, 5.2 Hz, 1H), |
| | | | 7.11 (br s, 1H), 7.07-7.00 (m, 2H), |
| | | | 6.99-6.91 (m, 3H), 4.20-4.14 (m, 2H), |
| | | | 4.09-4.04 (m, 2H), 2.01-1.94 (m, 4H). |
| 169 | 3-{4-[4-chloro-3- | 424.1 | ¹H NMR (400 MHz, Acetone) δ 7.56 (d, |
| | (trifluoromethyl)phenoxy]butoxy}- | | J = 8.8 Hz, 1H), 7.38 (br s, 1H), 7.33 (d, |
| | 2,6-difluorobenzamide | | J = 3.0 Hz, 1H), 7.25 (dd, J = 8.7, 2.8 Hz, |
| | | | 1H), 7.19 (td, J = 9.2, 5.2 Hz, 1H), |
| | | | 7.11 (br s, 1H), 6.95 (td, J = 9.0, 2.1 Hz, |
| | | | 1H), 4.23-4.15 (m, 4H), 2.03-1.97 (m, |
| | | | 4H). |
| 170 | 3-[4-(3,4- | 358.1 | ¹H NMR (400 MHz, Acetone) δ 7.39 (br |
| | difluorophenoxy)butoxy]-2,6- | | s, 1H), 7.28-7.15 (m, 2H), 7.11 (br s, |
| | difluorobenzamide | | 1H), 6.98-6.89 (m, 2H), 6.76 (dtd, J = 9.1, |
| | | | 3.3, 1.8 Hz, 1H), 4.20-4.14 (m, |
| | | | 2H), 4.12-4.07 (m, 2H), 2.00-1.95 (m, |
| | | | 4H). |
| 171 | 3-[4-(3,4- | 390.2 | ¹H NMR (400 MHz, DMSO) δ 8.09 (br |
| | dichlorophenoxy)butoxy]-2,6- | | s, 1H), 7.82 (br s, 1H), 7.51 (d, J = 8.9 Hz, |
| | difluorobenzamide | | 1H), 7.23 (d, J = 2.9 Hz, 1H), |
| | | | 7.26-7.18 (m, 1H), 7.05 (td, J = 9.0, 1.9 Hz, |
| | | | 1H), 6.96 (dd, J = 8.9, 2.9 Hz, 1H), |
| | | | 4.13-4.04 (m, 4H), 1.89-1.83 (m, 4H). |
| 172 | 3-[4-(3- | 400.2 | ¹H NMR (400 MHz, Acetone) δ 7.39 (br |
| | bromophenoxy)butoxy]-2,6- | | s, 1H), 7.23 (t, J = 8.0 Hz, 1H), 7.20 (td, |
| | difluorobenzamide | | 1H), 7.14-7.06 (m, 3H), 6.98-6.92 (m, |
| | | | 2H), 4.20-4.15 (m, 2H), 4.15-4.09 (m, |
| | | | 2H), 2.02-1.96 (m, 4H). |
| 173 | 3-[4-(3- | 347.1 | ¹H NMR (400 MHz, Acetone) δ |
| | cyanophenoxy)butoxy]-2,6- | | 7.52-7.46 (m, 1H), 7.38 (br s, 1H), |
| | difluorobenzamide | | 7.34-7.26 (m, 3H), 7.20 (td, J = 9.2, 5.2 Hz, 1H), |
| | | | 7.11 (br s, 1H), 6.96 (td, J = 9.0, 2.0 Hz, |
| | | | 1H), 4.21-4.16 (m, 4H), 2.03-1.98 (m, |
| | | | 4H). |
| 174 | 2,6-difluoro-3-{4-[3- | 448.0 | ¹H NMR (400 MHz, Acetone) δ 7.53 (t, J = 8.2 Hz, |
| | (pentafluoro-lambda~6~- | | 1H), 7.46-7.41 (m, 1H), |
| | sulfanyl)phenoxy]butoxy}benzamide | | 7.41-7.35 (m, 2H), 7.25 (dd, J = 8.3, 2.2 Hz, |
| | | | 1H), 7.20 (td, J = 9.2, 5.2 Hz, 1H), |
| | | | 7.12 (br s, 1H), 6.95 (td, J = 9.0, 2.0 Hz, 1H), |
| | | | 4.24-4.15 (m, 4H), 2.04-1.99 (m, 4H). |
| 175 | 3-[4-(4-tert- | 378.0 | ¹H NMR (400 MHz, Acetone) δ 7.39 (br |
| | butylphenoxy)butoxy]-2,6- | | s, 1H), 7.31 (d, J = 8.9 Hz, 2H), 7.20 (td, |
| | difluorobenzamide | | J = 9.2, 5.2 Hz, 1H), 7.11 (br s, 1H), |
| | | | 6.95 (td, J = 8.9, 2.1 Hz, 1H), 6.86 (d, J = 8.9 Hz, |
| | | | 2H), 4.19-4.15 (m, 2H), |
| | | | 4.08-4.03 (m, 2H), 2.02-1.92 (m, 4H), 1.28 (s, |
| | | | 9H). |
| 176 | 3-[4-(4- | 401.8 | ¹H NMR (400 MHz, Acetone) δ 8.09 (br |
| | bromophenoxy)butoxy]-2,6- | | s, 1H), 7.82 (br s, 1H), 7.43 (d, J = 9.1 Hz, |
| | difluorobenzamide | | 2H), 7.22 (td, J = 9.3, 5.3 Hz, 1H), |
| | | | 7.05 (td, J = 9.0, 1.9 Hz, 1H), 6.91 (d, J = 9.1 Hz, |
| | | | 2H), 4.10 (t, J = 5.9 Hz, 2H), |
| | | | 4.02 (t, J = 6.0 Hz, 2H), 1.89-1.83 (m, |
| | | | 4H). |
| 177 | 3-[4-(4- | 347.1 | ¹H NMR (400 MHz, Acetone) δ 7.69 (d, |
| | cyanophenoxy)butoxy]-2,6- | | J = 9.0 Hz, 2H), 7.39 (br s, 1H), 7.20 (td, |
| | difluorobenzamide | | J = 9.2, 5.2 Hz, 1H), 7.12 (br s, 1H), |
| | | | 7.12 (d, J = 9.0 Hz, 2H), 6.96 (td, J = 9.0, 2.0 Hz, |
| | | | 1H), 4.24-4.20 (m, 2H), |
| | | | 4.20-4.16 (m, 2H), 2.03-1.98 (m, 4H). |
| 178 | 2,6-difluoro-N′-hydroxy-3-(4- | 406.1 | ¹H NMR (400 MHz, DMSO-d₆) δ |
| | {[5-(trifluoromethyl)pyridin-2- | | 9.57 (s, 1H), 8.59-8.56 (m, 1H), 8.06 (dd, J = 8.8, |
| | yl]oxy}butoxy)benzenecarboximidamide | | 2.2 Hz, 1H), 7.23 (td, J = 9.3, 5.2 Hz, |
| | | | 1H), 7.04 (td, J = 9.1, 1.9 Hz, 1H), |
| | | | 7.01 (d, J = 8.8 Hz, 1H), 5.95 (s, 2H), |
| | | | 4.43-4.38 (m, 2H), 4.10 (t, J = 5.9 Hz, |
| | | | 2H), 1.94-1.81 (m, 4H). |
| 179 | 2,6-difluoro-3-[(4-{[5- | 390.0 | ¹H NMR (400 MHz, DMSO-d₆) δ |
| | (trifluoromethyl)pyridin-2- | | 8.60-8.54 (m, 1H), 8.05 (dd, J = 8.8, 2.3 Hz, |
| | yl]oxy}butyl)amino]benzamide | | 1H), 8.01 (br s, 1H), 7.70 (br s, 1H), |
| | | | 6.99 (d, J = 8.8 Hz, 1H), 6.88 (td, J = 8.9, 1.5 Hz, |
| | | | 1H), 6.69 (td, J = 9.4, 5.5 Hz, 1H), |
| | | | 5.43 (td, J = 5.6, 1.8 Hz, 1H), 4.37 (t, J = 6.5 Hz, |
| | | | 2H), 3.12 (q, J = 6.7 Hz, 2H), |
| | | | 1.85-1.76 (m, 2H), 1.73-1.61 (m, 2H). |
| 180 | 2,6-difluoro-3-[methyl(4-{[5- | 404.0 | ¹H NMR (400 MHz, Chloroform-d) δ |
| | (trifluoromethyl)pyridin-2- | | 8.43-8.39 (m, 1H), 7.75 (dd, J = 8.7, |
| | yl]oxy}butyl)amino]benzamide | | 2.5 Hz, 1H), 7.04-6.92 (m, 1H), |
| | | | 6.85 (td, J = 9.0, 1.7 Hz, 1H), 6.79 (d, J = 8.7 Hz, |
| | | | 1H), 6.03-5.84 (m, 2H), 4.36 (t, J = 6.4 Hz, |
| | | | 2H), 3.13 (t, J = 7.3 Hz, 2H), |
| | | | 2.80 (s, 3H), 1.86-1.77 (m, 2H), |
| | | | 1.76-1.65 (m, 2H). |
| 181 | 2,6-difluoro-3-[(2- | 449.2 | ¹H NMR (400 MHz, Acetone-d₆) δ |
| | hydroxyethyl){4-[4- | | 7.41 (br s, 1H), 7.23 (d, J = 9.1 Hz, 3H), |
| | (trifluoromethoxy)phenoxy]butyl}amino]benzamide | | 7.19 (td, 1H), 7.08 (br s, 1H), 7.00 (d, J = 9.1 Hz, |
| | | | 2H), 6.90 (td, J = 8.8, 1.7 Hz, 1H), |
| | | | 4.03 (t, J = 6.4 Hz, 2H), 3.66-3.55 (m, |
| | | | 3H), 3.32-3.24 (m, 4H), 1.86-1.77 (m, |
| | | | 2H), 1.72-1.62 (m, 2H). |
| 182 | 3-[(3-cyanopropyl){4-[4- | 472.1 | ¹H NMR (400 MHz, Chloroform-d) δ |
| | (trifluoromethoxy)phenoxy]butyl}amino]- | | 7.14-7.10 (m, 2H), 7.11-7.05 (m, 1H), |
| | 2,6- | | 6.89 (td, J = 8.9, 1.4 Hz, 1H), 6.84 (d, J = 9.2 Hz, |
| | difluorobenzamide | | 2H), 5.98 (br s, 1H), 5.91 (br s, |
| | | | 1H), 3.93 (t, J = 6.2 Hz, 2H), 3.22 (t, J = 6.6 Hz, |
| | | | 2H), 3.12 (t, J = 7.3 Hz, 2H), |
| | | | 2.41 (t, J = 7.0 Hz, 2H), 1.78 (dt, J = 12.4, |
| | | | 6.4 Hz, 4H), 1.68-1.60 (m, 2H). |
| 183 | 3-[(2-amino-2-oxoethyl){4-[4- | 462.1 | ¹H NMR (400 MHz, Chloroform-d) δ |
| | (trifluoromethoxy)phenoxy]butyl}amino]- | | 7.19-7.09 (m, 3H), 6.91 (td, J = 9.0, 1.8 Hz, |
| | 2,6- | | 1H), 6.83 (d, J = 9.2 Hz, 2H), |
| | difluorobenzamide | | 6.77 (br s, 1H), 5.98 (br s, 2H), 5.53 (br s, |
| | | | 1H), 3.92 (t, J = 6.1 Hz, 2H), 3.69 (s, |
| | | | 2H), 3.21-3.15 (m, 2H), 1.83-1.75 (m, |
| | | | 2H), 1.71-1.63 (m, 2H). |
| 184 | 2,6-difluoro-3-[(2- | 463.2 | ¹H NMR (400 MHz, Chloroform-d) δ |
| | methoxyethyl){4-[4- | | 7.12 (d, J = 9.1 Hz, 2H), 7.07 (dt, J = 9.2, |
| | (trifluoromethoxy)phenoxy]butyl}amino]benzamide | | 4.6 Hz, 1H), 6.89-6.85 (m, 1H), |
| | | | 6.84 (d, J = 9.2 Hz, 2H), 5.99 (br s, 1H), |
| | | | 5.95 (br s, 1H), 3.93 (t, J = 6.3 Hz, 2H), |
| | | | 3.46 (t, J = 5.8 Hz, 2H), 3.31 (t, J = 6.0 Hz, |
| | | | 2H), 3.29 (s, 3H), 3.24 (t, J = 7.2 Hz, |
| | | | 2H), 1.83-1.73 (m, 2H), 1.66 (d, J = 8.0 Hz, |
| | | | 2H). |
| 185 | 2,6-difluoro-3-([(6- | 510.2 | ¹H NMR (400 MHz, Chloroform-d) δ |
| | methylpyridin-3-yl)methyl]{4- | | 8.39 (s, 1H), 7.56 (d, J = 7.8 Hz, 1H), |
| | [4- | | 7.17-7.05 (m, 3H), 6.98 (td, J = 9.1, 5.7 Hz, |
| | (trifluoromethoxy)phenoxy]butyl}amino)benzamide | | 1H), 6.89-6.76 (m, 3H), 5.98 (br s, |
| | | | 1H), 5.91 (br s, 1H), 4.21 (s, 2H), 3.86 (t, |
| | | | J = 6.1 Hz, 2H), 3.10 (t, J = 7.1 Hz, 2H), |
| | | | 2.54 (s, 3H), 1.80-1.68 (m, 2H), |
| | | | 1.69-1.57 (m, 2H). |
| 186 | ethyl N-(3-carbamoyl-2,4- | 491.1 | ¹H NMR (400 MHz, Chloroform-d) δ |
| | difluorophenyl)-N-{4-[4- | | 7.17-7.09 (m, 3H), 6.86 (td, J = 9.1, 1.8 Hz, |
| | (trifluoromethoxy)phenoxy]butyl}glycinate | | 1H), 6.84 (d, J = 9.2 Hz, 2H), |
| | | | 5.92 (br s, 1H), 5.85 (br s, 1H), 4.13 (q, J = 7.1 Hz, |
| | | | 2H), 3.99-3.90 (m, 4H), 3.33 (t, |
| | | | J = 7.1 Hz, 2H), 1.89-1.77 (m, 2H), |
| | | | 1.77-1.64 (m, 2H), 1.23 (t, J = 7.1 Hz, |
| | | | 3H). |
| 187 | N-(3-carbamoyl-2,4- | 460.7 | ¹H NMR (400 MHz, Acetone-d₆) δ |
| | difluorophenyl)-N-{4-[4- | (−ve | 8.01 (s, 0H), 7.18 (d, J = 8.9 Hz, 2H), |
| | (trifluoromethoxy)phenoxy]butyl}glycine | mode) | 7.04 (td, J = 9.4, 5.9 Hz, 1H), 6.99-6.90 (m, |
| | | | 2H), 6.78 (t, J = 8.8 Hz, 1H), 3.94 (t, J = 6.3 Hz, |
| | | | 2H), 3.56 (s, 2H), 3.26 (t, J = 7.0 Hz, |
| | | | 2H), 1.72 (q, J = 6.9, 6.2 Hz, 2H), |
| | | | 1.63 (d, J = 6.4 Hz, 2H). |

Additionally prepared, in accordance with methods known in the art or by reference to the methods described herein, were further compounds of Formula (I) 188-213 as provided below.

| Cpd | | |
|---|---|---|
| No | Structure | Name (ACD) |
| 188 | 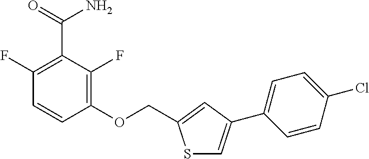 [SMILES: NC(=O)c1c(F)ccc(OCc2cc(-c3ccc(Cl)cc3)cs2)c1F] | 3-{[4-(4-chlorophenyl)thiophen-2-yl]methoxy}-2,6- difluorobenzamide |
| 189 | 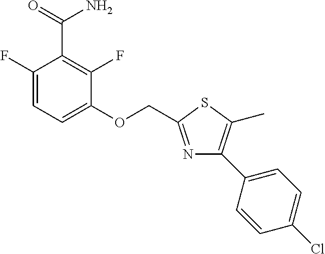 [SMILES: Cc1sc(COc2ccc(F)c(C(N)=O)c2F)nc1-c1ccc(Cl)cc1] | 3-{[4-(4-chlorophenyl)-5-methyl-1,3-thiazol-2- yl]methoxy}-2,6-difluorobenzamide |
| 190 | 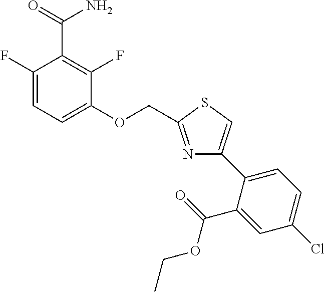 [SMILES: CCOC(=O)c1cc(Cl)ccc1-c1csc(COc2ccc(F)c(C(N)=O)c2F)n1] | ethyl 2-{2-[(3-carbamoyl-2,4- difluorophenoxy)methyl]-1,3-thiazol-4-yl}-5- chlorobenzoate |
| 191 | 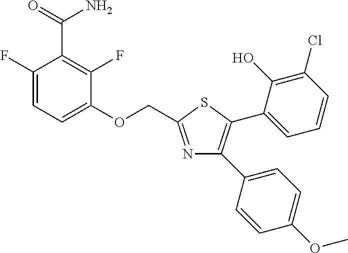 [SMILES: COc1ccc(-c2nc(COc3ccc(F)c(C(N)=O)c3F)sc2-c2cccc(Cl)c2O)cc1] | 3-{[5-(3-chloro-2-hydroxyphenyl)-4-(4- methoxyphenyl)-1,3-thiazol-2-yl]methoxy}-2,6- difluorobenzamide |
| 192 | 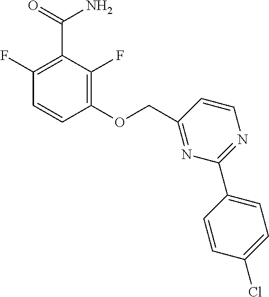 [SMILES: NC(=O)c1c(F)ccc(OCc2ccnc(-c3ccc(Cl)cc3)n2)c1F] | 3-{[2-(4-chlorophenyl)pyrimidin-4-yl]methoxy}-2,6- difluorobenzamide |
| 193 | 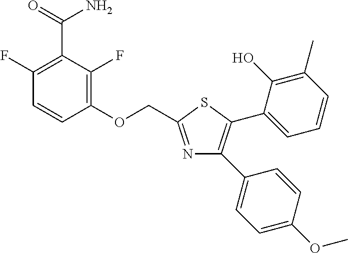 [SMILES: COc1ccc(-c2nc(COc3ccc(F)c(C(N)=O)c3F)sc2-c2cccc(C)c2O)cc1] | 2,6-difluoro-3-{[5-(3-fluoro-2-hydroxyphenyl)-4-(4- methoxyphenyl)-1,3-thiazol-2-yl]methoxy}benzamide |
| 194 | 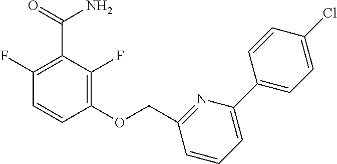 [SMILES: NC(=O)c1c(F)ccc(OCc2cccc(-c3ccc(Cl)cc3)n2)c1F] | 3-{[6-(4-chlorophenyl)pyridin-2-yl]methoxy}-2,6- difluorobenzamide |
| 195 | 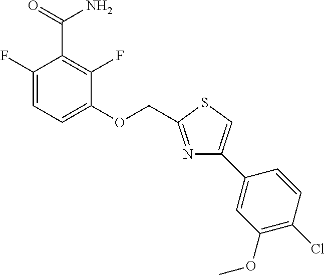 [SMILES: COc1cc(-c2csc(COc3ccc(F)c(C(N)=O)c3F)n2)ccc1Cl] | 3-{[4-(4-chloro-3-methoxyphenyl)-1,3-thiazol-2- yl]methoxy}-2,6-difluorobenzamide |
| 196 | 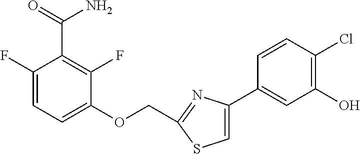 [SMILES: NC(=O)c1c(F)ccc(OCc2nc(-c3ccc(Cl)c(O)c3)cs2)c1F] | 3-{[4-(4-chloro-3-hydroxyphenyl)-1,3-thiazol-2- yl]methoxy}-2,6-difluorobenzamide |
| 197 | 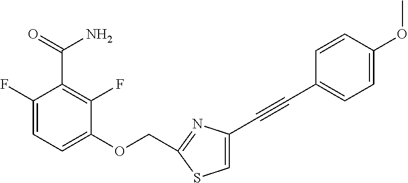 [SMILES: COc1ccc(C#Cc2csc(COc3ccc(F)c(C(N)=O)c3F)n2)cc1] | 2,6-difluoro-3-({4-[(4-methoxyphenyl)ethynyl]-1,3- thiazol-2-yl}methoxy)benzamide |
| 198 | 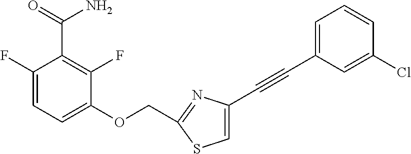 [SMILES: NC(=O)c1c(F)ccc(OCc2nc(C#Cc3cccc(Cl)c3)cs2)c1F] | 3-({4-[(3-chlorophenyl)ethynyl]-1,3-thiazol-2- yl}methoxy)-2,6-difluorobenzamide |
| 199 | 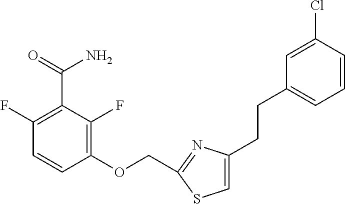 [SMILES: NC(=O)c1c(F)ccc(OCc2nc(CCc3cccc(Cl)c3)cs2)c1F] | 3-({4-[2-(3-chlorophenyl)ethyl]-1,3-thiazol-2- yl}methoxy)-2,6-difluorobenzamide |
| 200 | 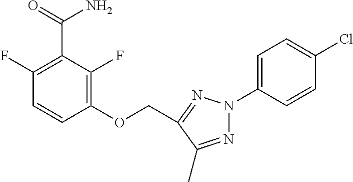 [SMILES: Cc1nn(-c2ccc(Cl)cc2)nc1COc1ccc(F)c(C(N)=O)c1F] | 3-{[2-(4-chlorophenyl)-5-methyl-2H-1,2,3-triazol-4- yl]methoxy}-2,6-difluorobenzamide |
| 201 | 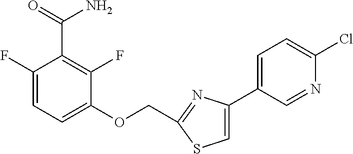 [SMILES: NC(=O)c1c(F)ccc(OCc2nc(-c3ccc(Cl)nc3)cs2)c1F] | 3-{[4-(6-chloropyridin-3-yl)-1,3-thiazol-2- yl]methoxy}-2,6-difluorobenzamide |
| 202 | 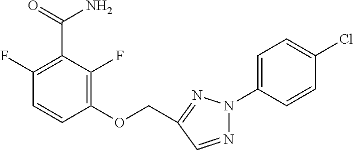 [SMILES: NC(=O)c1c(F)ccc(OCc2cnn(-c3ccc(Cl)cc3)n2)c1F] | 3-{[2-(4-chlorophenyl)-2H-1,2,3-triazol-4- yl]methoxy}-2,6-difluorobenzamide |
| 203 | 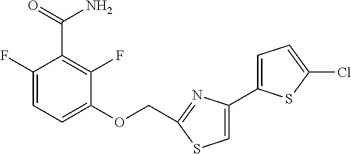 [SMILES: NC(=O)c1c(F)ccc(OCc2nc(-c3ccc(Cl)s3)cs2)c1F] | 3-{[4-(5-chlorothiophen-2-yl)-1,3-thiazol-2- yl]methoxy}-2,6-difluorobenzamide |
| 204 | 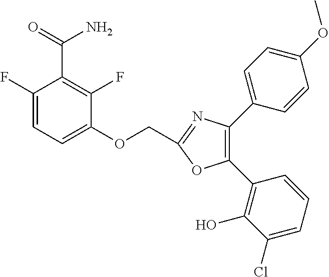 [SMILES: COc1ccc(-c2nc(COc3ccc(F)c(C(N)=O)c3F)oc2-c2cccc(Cl)c2O)cc1] | 3-{[5-(3-chloro-2-hydroxyphenyl)-4-(4- methoxyphenyl)-1,3-oxazol-2-yl]methoxy}-2,6- difluorobenzamide |
| 205 | 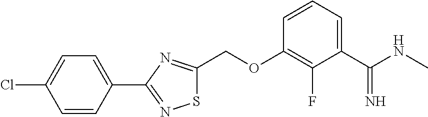 [SMILES: CNC(=N)c1cccc(OCc2nc(-c3ccc(Cl)cc3)ns2)c1F] | 3-{[3-(4-chlorophenyl)-1,2,4-thiadiazol-5- yl]methoxy}-2-fluoro-N- methylbenzenecarboximidamide |
| 206 | 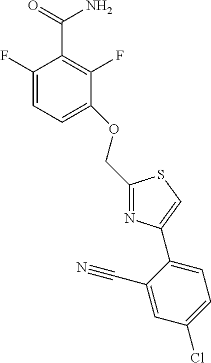 [SMILES: N#Cc1cc(Cl)ccc1-c1csc(COc2ccc(F)c(C(N)=O)c2F)n1] | 3-{[4-(4-chloro-2-cyanophenyl)-1,3-thiazol-2- yl]methoxy}-2,6-difluorobenzamide |
| 207 | 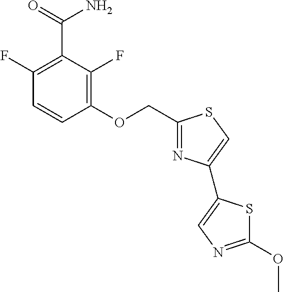 [SMILES: COc1ncc(-c2csc(COc3ccc(F)c(C(N)=O)c3F)n2)s1] | 2,6-difluoro-3-[(2′-methoxy-4,5′-bi-1,3-thiazol-2- yl)methoxy]benzamide |
| 208 | 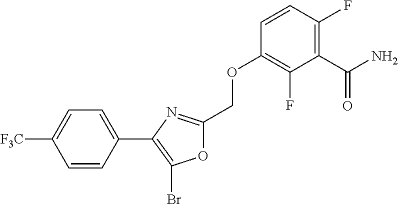 [SMILES: NC(=O)c1c(F)ccc(OCc2nc(-c3ccc(C(F)(F)F)cc3)c(Br)o2)c1F] | 3-({5-bromo-4-[4-(trifluoromethyl)phenyl]-1,3-oxazol- 2-yl}methoxy)-2,6-difluorobenzamide |
| 209 | 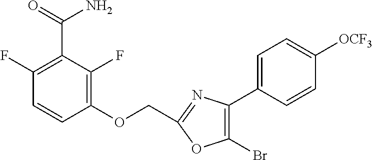 [SMILES: NC(=O)c1c(F)ccc(OCc2nc(-c3ccc(OC(F)(F)F)cc3)c(Br)o2)c1F] | 3-({5-bromo-4-[4-(trifluoromethoxy)phenyl]-1,3- oxazol-2-yl}methoxy)-2,6-difluorobenzamide |
| 210 | 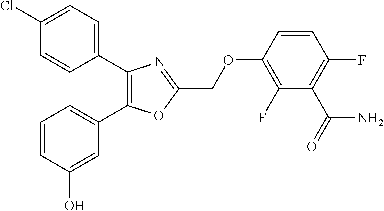 [SMILES: NC(=O)c1c(F)ccc(OCc2nc(-c3ccc(Cl)cc3)c(-c3cccc(O)c3)o2)c1F] | 3-{[4-(4-chlorophenyl)-5-(3-hydroxyphenyl)-1,3- oxazol-2-yl]methoxy}-2,6-difluorobenzamide |
| 211 | 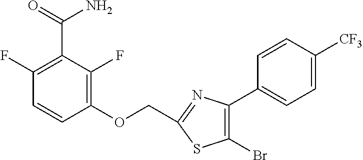 [SMILES: NC(=O)c1c(F)ccc(OCc2nc(-c3ccc(C(F)(F)F)cc3)c(Br)s2)c1F] | 3-({5-bromo-4-[4-(trifluoromethyl)phenyl]-1,3-thiazol- 2-yl}methoxy)-2,6-difluorobenzamide |
| 212 | 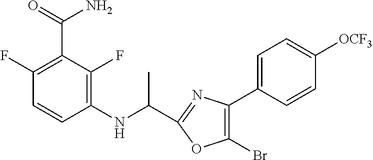 [SMILES: CC(Nc1ccc(F)c(C(N)=O)c1F)c1nc(-c2ccc(OC(F)(F)F)cc2)c(Br)o1] | 3-[(1-{5-bromo-4-[4-(trifluoromethoxy)phenyl]-1,3- oxazol-2-yl}ethyl)amino]-2,6-difluorobenzamide |
| 213 | 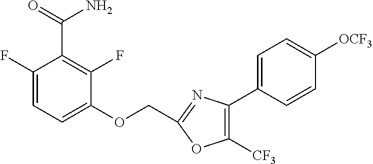 [SMILES: NC(=O)c1c(F)ccc(OCc2nc(-c3ccc(OC(F)(F)F)cc3)c(C(F)(F)F)o2)c1F] | 2,6-difluoro-3-({4-[4-(trifluoromethoxy)phenyl]-5- (trifluoromethyl)-1,3-oxazol-2-yl}methoxy)benzamide |

### Example of Chiral HPLC Method for the Separation of Enantiomers

The chiral HPLC conditions used to separate Compound 32 are as follows:

Preparative Method:

Column: CHIRALPAK® AD-H, (250×30) mm, 5 μm

Mobile phase: n-Hexane:EtOH:MeOH (80:10:10 v/v/v)

Flow rate: 40 ml/min

Detection: UV 265 nm

Temperature: 25° C.

Diluent: Mobile phase

Feed Conc.: 9.0 mg/ml

Inj Volume: 7 ml (on column: 63 mg)

Analytical Method:

Column: CHIRALPAK® IA (250×4.6) mm, 5 m

Mobile phase: n-Hexane:EtOH (80:20 v/v)

Flow rate: 1.0 ml/min

Detection: DAD 265 nm

Temperature: 25° C.

Isomer-A: 9.20 min (Isomeric purity 99.30%)

Isomer-B (Compound 47): 9.90 min (Isomeric purity 99.60%) was determined to be the

(R)-enantiomer with absolute configuration as follows:

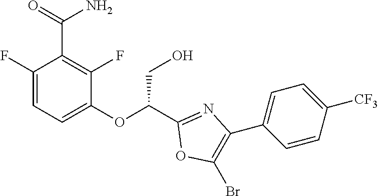 [SMILES: NC(=O)c1c(F)ccc(O[C@H](CO)c2nc(-c3ccc(C(F)(F)F)cc3)c(Br)o2)c1F]

### Biological Data

The in vitro antiviral activity of the compounds of the invention may be determined using the following protocols.

Bacterial Assay: Determination of Antibacterial Activity Compounds of the invention were tested for antimicrobial activity by susceptibility testing in liquid or on solid media. Minimum inhibitory concentrations (MICs) for compounds against each strain were determined by the broth microdilution or agar dilution method according to the guidelines of the Clinical Laboratories and Standards Institute, formerly the National Committee for Clinical Laboratory Standards (Clinical Laboratories and Standards Institute. Methods for Dilution Antimicrobial Susceptibility Tests for Bacteria That Grow Anaerobically; Approved Standard-Seventh Edition. Document M11-A7, CLSI, Wayne, Pa., 2007, Clinical Laboratories and Standards Institute). Briefly MICs were determined by visible inspection after 48 hours of growth in the presence of compounds, cultures were grown at 37° C. in anaerobic gas packs using reinforced clostridial medium supplemented with 0.1% defribrinated horse blood. The bacterial strains tested include C. difficile (Clostridium difficile (Isolate ID ATCC 9689)) (Table 2) and two drug resistant clinical isolates (Clostridium difficile (Isolate ID BI-9) and Clostridium difficile (Isolate ID 027-01) (Table 3).

MICs were determined to be within the activity ranges “A” activity≦1.01 μg/mL; “B” activity>1.0 μg/mL-4.0 μg/mL; “C” activity>4.0 μg/mL-16 μg/mL

TABLE 4
Antibacterial activity of compounds against C. difficile

| | C. difficile | | C. difficile | | C. difficile |
|---|---|---|---|---|---|
| | MIC | Com- | MIC | | MIC |
| Compound | Activity | pound | Activity | Compound | Activity |
| No. | Range | No. | Range | No. | Range |
| 1 | A | 2 | A | 3 | A |
| 4 | B | 5 | A | 6 | A |
| 7 | A | 8 | A | 9 | A |
| 10 | B | 11 | A | 12 | A |
| 13 | B | 14 | C | 15 | B |
| 16 | A | 17 | A | 18 | B |
| 19 | A | 20 | A | 21 | A |
| 22 | A | 23 | C | 24 | A |
| 25 | C | 26 | C | 27 | B |
| 28 | C | 29 | A | 30 | A |
| 31 | A | 32 | A | 33 | A |
| 34 | A | 35 | A | 36 | A |
| 37 | C | 38 | A | 39 | A |
| 40 | A | 41 | B | 42 | A |
| 43 | B | 44 | C | 45 | B |
| 46 | B | 47 | A | 48 | B |
| 49 | B | 50 | A | 51 | A |
| 52 | A | 53 | B | 54 | B |
| 55 | B | 56 | B | 57 | A |
| 58 | C | 59 | A | 60 | A |
| 61 | B | 62 | B | 63 | A |
| 64 | A | 65 | B | 66 | A |
| 67 | B | 68 | B | 69 | A |
| 70 | A | 71 | C | 72 | A |
| 73 | A | 74 | A | 75 | B |
| 76 | A | 77 | B | 78 | A |
| 79 | C | 80 | A | 81 | A |
| 82 | A | 83 | A | 84 | A |
| 85 | B | 86 | B | 87 | B |
| 88 | B | 89 | A | 90 | A |
| 91 | A | 92 | B | 93 | B |
| 94 | B | 95 | A | 96 | A |
| 97 | B | 98 | B | 99 | A |
| 100 | B | 101 | B | 102 | B |
| 103 | C | 104 | A | 105 | A |
| 106 | A | 107 | C | 108 | A |
| 109 | B | 110 | A | 111 | A |
| 112 | A | 113 | B | 114 | B |
| 115 | B | 116 | B | 117 | A |
| 118 | A | 119 | B | 120 | C |
| 121 | B | 122 | A | 123 | B |
| 124 | B | 125 | B | 126 | A |
| 127 | A | 128 | A | 129 | A |
| 130 | A | 131 | A | 132 | A |
| 133 | A | 134 | A | 135 | A |
| 136 | B | 137 | A | 138 | A |
| 139 | C | 140 | B | 141 | A |
| 142 | B | 143 | A | 144 | B |
| 145 | A | 146 | A | 147 | B |
| 148 | A | 149 | A | 150 | B |
| 151 | B | 152 | A | 153 | A |
| 154 | B | 155 | A | 156 | B |
| 157 | B | 158 | C | 159 | A |
| 160 | A | 161 | A | 162 | B |
| 163 | A | 164 | A | 165 | B |
| 166 | A | 167 | B | 168 | B |
| 169 | A | 170 | B | 171 | A |
| 172 | B | 173 | C | 174 | A |
| 175 | A | 176 | A | 177 | B |
| 178 | C | 179 | C | 180 | B |
| 181 | B | 182 | C | 183 | C |
| 184 | B | 185 | C | 186 | B |
| 187 | C | 188 | A | 189 | A |
| 190 | C | 191 | C | 192 | A |
| 193 | C | 194 | A | 195 | C |
| 196 | B | 197 | A | 198 | A |
| 199 | A | 200 | B | 201 | B |
| 202 | B | 203 | A | 204 | C |
| 205 | C | 206 | A | 207 | B |
| 208 | A | 209 | A | 210 | C |
| 211 | B | 212 | B | 213 | A |

TABLE 5
Antibacterial activity of selected compounds against
virulent C. difficile NAP1/BI/027 strains

| Compound | MIC (μg/mL) | MIC (μg/mL) |
|---|---|---|
| No. | C. difficile (B-I9) | C. difficile (027-01) |
| 1 | 0.12 | 0.12 |
| 7 | 0.12 | 0.12 |
| 8 | 0.25 | 0.5 |
| 9 | 0.5 | 1 |
| 10 | 2 | 4 |
| 11 | 0.25 | 0.25 |
| 12 | 1 | 1 |
| 16 | 0.5 | 0.5 |
| 17 | 0.25 | 0.185 |
| 19 | 2 | 1 |
| 20 | 0.06 | 0.06 |
| 21 | 5 | 5 |
| 33 | 0.5 | 0.12 |
| 47 | 0.12 | 0.12 |
| 49 | 2 | 2 |
| 57 | 0.5 | 0.5 |
| 74 | 0.5 | 0.5 |
| 80 | 0.5 | 0.5 |
| 102 | 2 | 2 |
| 104 | 0.12 | 0.12 |
| 105 | 1 | 0.5 |
| 106 | 0.5 | 0.5 |
| 108 | 0.5 | 0.5 |
| 110 | 0.06 | 0.06 |
| 111 | 0.25 | 0.25 |
| 112 | 0.12 | 0.12 |
| 126 | 0.5 | 0.5 |
| 127 | 0.25 | 0.25 |
| 128 | 0.12 | 0.06 |
| 129 | 0.06 | 0.06 |
| 130 | 0.5 | 0.5 |
| 131 | 0.25 | 0.25 |
| 132 | 0.5 | 0.25 |
| 133 | 0.5 | 0.5 |
| 134 | 0.25 | 0.25 |
| 135 | 0.5 | 0.5 |
| 136 | 4 | 4 |
| 152 | 0.5 | 0.5 |
| 155 | 0.12 | 0.12 |
| 159 | 0.12 | 0.12 |
| 160 | 0.25 | 0.25 |
| 161 | 1 | 2 |
| 189 | 1 | 1 |
| 192 | 1 | 1 |
| 194 | 0.5 | 0.5 |
| 198 | 0.25 | 0.12 |
| 203 | 1 | 0.5 |
| 205 | 16 | 16 |
| 207 | 4 | 4 |
| 208 | 0.06 | 0.06 |
| 209 | 0.25 | 0.12 |
| 213 | 0.25 | 0.12 |

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication, or information derived from it, or to any matter which is known, is not, and should not be taken as an acknowledgement or admission or any form of suggestion that that prior publication, or information derived from it, or known matter forms part of the common general knowledge in the field of endeavour to which this specification relates.

1. A method for treating a Clostridium difficile infection comprising administration of a compound of Formula (I″): 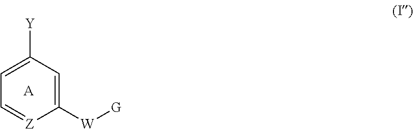
to a patient with said infection or a pharmaceutically acceptable salt, racemate, isomer, diastereoisomer, enantiomer, hydrate, solvate, N-oxide, pharmaceutically acceptable derivative or prodrug thereof,
wherein:
A ring is optionally substituted with one or more substituents;
G is, 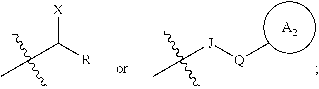
Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;
Z is CH or N;
W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and
R is optionally substituted and is selected from C₁₋₁₂alkyl, C₂₋₁₂alkenyl, C₂₋₁₂alkynyl, C₃₋₈cycloalkyl, C₆₋₁₀aryl and 4-10-membered heterocycles;
Q is selected from O, CH₂ or NR₇ where R₇ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
J is an optionally substituted linker selected from C₁₋₁₂alkyl, C₂₋₁₂alkenyl, C₂₋₁₂alkynyl, optionally interrupted by an ether linkage; and
A₂ is optionally substituted and is selected from C₆₋₁₀aryl and 5-10-membered heterocycles.

2. The method of claim 1 wherein the compound of Formula (I″) is a compound of Formula (I) or Formula (II): 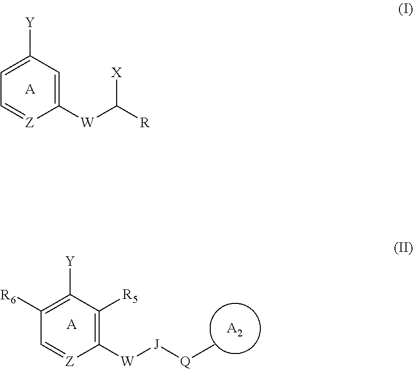
or a pharmaceutically acceptable salt, racemate, isomer, diastereoisomer, enantiomer, hydrate, solvate, N-oxide, or prodrug thereof, wherein:
A ring is optionally substituted with one or more substituents;
Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂Cl-6alkyl or R₃ joins together with R₁ or R₂ to form a —C(═O)—O— cyclic linking unit;
Z is CH or N;
W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and
R is optionally substituted and is selected from C₁₋₁₂alkyl, C₂₋₁₂alkenyl, C₂₋₁₂alkynyl, C₃₋₈cycloalkyl, C₆₋₁₀aryl and 4-10-membered heterocycles;
R₅ is selected from F or C1;
R₆ is H or an optional substituent;
Q is selected from O, CH₂ or NR₇ where R₇ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
J is an optionally substituted linker selected from C₁₋₁₂alkyl, C₂₋₁₂alkenyl, C₂₋₁₂alkynyl, optionally interrupted by an ether linkage; and
A2 is optionally substituted and is selected from C₆₋₁₀aryl and 5-10-membered heterocycles.

3. The method of claim 1 wherein the compound of Formula (I″) is a compound of Formula (I): 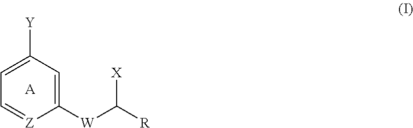
or a pharmaceutically acceptable salt, racemate, isomer, diastereoisomer, enantiomer, hydrate, solvate, N-oxide, pharmaceutically acceptable derivative or prodrug thereof,
wherein
A ring is optionally substituted with one or more substituents;
Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;
Z is CH or N;
W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and
R is optionally substituted and is selected from C₁₋₁₂alkyl, C₂₋₁₂alkenyl, C₂₋₁₂alkynyl, C₃₋₈ cycloalkyl, C₆₋₁₀aryl and 4-10-membered heterocycles.

4-17. (canceled)

18. The method of claim 1, wherein W is O or NR₄ where R₄ is H or is a C₁₋₁₂alkyl optionally substituted with one or more groups selected from hydroxyl, nitrile, —CONR^{A}R^{B}, (C₁-C₆)alkoxy, monocyclic heteroaryl and COOR^{A}, wherein the monocyclic heteroaryl is optionally substituted with one or more C₁-C₆alkyl groups and wherein R^{A} and R^{B} are independently hydrogen or a (C₁-C₆)alkyl.

19-32. (canceled)

33. The method of claim 1, wherein Q is selected from O or NR₇ where R₇ is H.

34-37. (canceled)

38. The method of claim 1, wherein J is a linker selected from C₁₋₁₂alkyl, optionally interrupted by an ether linkage.

39-44. (canceled)

45. A method for treating a Clostridium difficile infection comprising administration of a compound of Formula (I) of claim 1 to a patient with said infection, wherein the compound is selected from any one of compounds 1 to 213 or a salt, racemate, isomer, diastereoisomer, enantiomer, hydrate, solvate, N-oxide, pharmaceutically acceptable derivative or prodrug thereof.

46-47. (canceled)

48. A compound of formula (Ia) 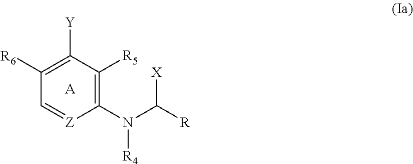
or a salt, racemate, isomer, diastereoisomer, enantiomer, hydrate, solvate, N-oxide, pharmaceutically acceptable derivative or prodrug thereof,
wherein
A ring is optionally substituted with one or more substituents;
Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;
Z is CH or N;
R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
R₅ and R₆ are independently selected from F or C1;
X is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles; and
R is optionally substituted and is selected from C₁₋₁₂alkyl, C₂₋₁₂alkenyl, C₂₋₁₂alkynyl, C₃₋₈cycloalkyl, C₆₋₁₀aryl and 4-10-membered heterocycles.

49-54. (canceled)

55. The compound of claim 48, wherein R₄ is H or is a C₁₋₁₂alkyl optionally substituted with one or more groups selected from hydroxyl, nitrile, —CONR^{A}R^{B}, (C₁-C₆)alkoxy, monocyclic heteroaryl and COOR^{A}, wherein the monocyclic heteroaryl is optionally substituted with one or more C₁-C₆alkyl groups and wherein R^{A} and R^{B} are independently hydrogen or a (C₁-C₆)alkyl.

56-61. (canceled)

62. The compound of claim 48, wherein R is an optionally substituted with (C₁-C₆)alkyl, (C₂-C₆)alkenyl, (C₂-C₆)alkynyl, (C₁-C₆)alkoxy, hydroxy, hydroxy(C₁-C₆)alkyl, (C₁-C₃)alkoxy(C₁-C₃)alkyl, mercapto, mercapto(C₁-C₆)alkyl, (C₁-C₆)alkylthio, halo, fully or partially fluorinated (C₁-C₃)alkyl, (C₁-C₃)alkoxy or (C₁-C₃)alkylthio, nitro, nitrile (—CN), oxo (═O), thiols, alkylthiols, trialkylsilyl, diarylalkylsilyl, trialkylsilyloxy, diarylalkylsilyloxy, dialkylphosphonyl, dialkoxyphosphonyl, diarylphosphonyl, diaryloxyphosphonyl, alkylphosphinyl, arylphosphinyl, alkoxyphosphinyl, aryloxyphosphinyl, dialkyoxyphoshoryl, diaryloxyphosphoryl, phosphoryl, phosphinyl, phenyl, phenyl(C₁-C₃)alkyl-, phenoxy, monocyclic heteroaryl, heteroaryl(C₁-C₃)alkyl-, or heteroaryloxy with 5 or 6 ring atoms, cycloalkyl having 3 to 6 ring carbon atoms, —COOR^{A}, —COR^{A}, —OCOR^{A}, —SO₂R^{A}, —CONR^{A}R^{B}, —CONHNH₂, —SO₂NR^{A}R^{B}, SF₅, —NR^{A}R^{B}, —NHNH₂, —OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl, hydroxy(C₁-C₆)alkyl, or (C₁-C₃)alkoxy(C₁-C₃)alkyl group or, in the case where R^{A} and R^{B} are linked to the same N atom, R^{A} and R^{B} taken together with that nitrogen may form a cyclic amino ring such as morpholinyl, piperidinyl, piperazinyl, or 4-(C₁-C₆)alkyl-piperizinyl, wherein each optional substituent may also be optionally substituted.

63. (canceled)

64. The compound of claim 48 which is a compound of compound number 122, 123, 150, 152, 179-187 or 212 or a salt, racemate, isomer, diastereoisomer, enantiomer, hydrate, solvate, N-oxide, pharmaceutically acceptable derivative or prodrug thereof.

65. A compound of formula (II) 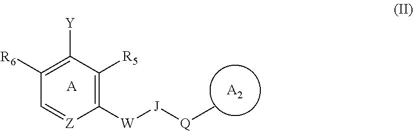
or a salt, racemate, isomer, diastereoisomer, enantiomer, hydrate, solvate, N-oxide, pharmaceutically acceptable derivative or prodrug thereof,
wherein
A ring is optionally substituted with one or more substituents;
Y is selected from CONR₁R₂ and C(═NR₃)NR₁R₂ where R₁ and R₂ are independently selected from H or optionally substituted C₁₋₆alkyl and R₃ is selected from H, OH, OC₁₋₆alkyl, OC(═O)C₁₋₆alkyl, SO₂C₁₋₆alkyl or R₃ joins together with R₁ or R₂ to form a —C(═O)—O— cyclic linking unit;
R₅ is selected from F or Cl;
R₆ is H or an optional substituent;
Z is CH or N;
W is O or NR₄ where R₄ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
Q is selected from O, CH₂ or NR₇ where R₇ is H or is optionally substituted and selected from C₁₋₁₂alkyl, C₂₋₁₂alkenyl, C₂₋₁₂alkynyl, C₃₋₈cycloalkyl, C₆₋₁₀aryl and 4-10-membered heterocycles;
J is an optionally substituted linker selected from C₁₋₁₂alkyl, C₂₋₁₂alkenyl, C₂₋₁₂alkynyl, optionally interrupted by an ether linkage; and
A₂ is optionally substituted and is selected from C₆₋₁₀aryl and 5-10-membered heterocycles.

66-73. (canceled)

74. The compound of claim 65, wherein W is O or NR₄ where R₄ is H or is a C₁₋₁₂alkyl optionally substituted with one or more groups selected from hydroxyl, nitrile, —CONR^{A}R^{B}, (C₁-C₆)alkoxy, monocyclic heteroaryl and COOR^{A}, wherein the monocyclic heteroaryl is optionally substituted with one or more C₁-C₆alkyl groups and wherein R^{A} and R^{B} are independently hydrogen or a (C₁-C₆)alkyl.

75-83. (canceled)

84. The compound of claim 65, wherein A₂ is an optionally substituted 5-10-membered heterocycle.

85. The compound of claim 65, wherein A₂ is an optionally substituted with (C₁-C₆)alkyl, (C₂-C₆)alkenyl, (C₂-C₆)alkynyl, (C₁-C₆)alkoxy, hydroxy, hydroxy(C₁-C₆)alkyl, (C₁-C₃)alkoxy(C₁-C₃)alkyl, mercapto, mercapto(C₁-C₆)alkyl, (C₁-C₆)alkylthio, halo, fully or partially fluorinated (C₁-C₃)alkyl, (C₁-C₃)alkoxy or (C₁-C₃)alkylthio, nitro, nitrile (—CN), oxo (═O), thiols, alkylthiols, trialkylsilyl, diarylalkylsilyl, trialkylsilyloxy, diarylalkylsilyloxy, dialkylphosphonyl, dialkoxyphosphonyl, diarylphosphonyl, diaryloxyphosphonyl, alkylphosphinyl, arylphosphinyl, alkoxyphosphinyl, aryloxyphosphinyl, dialkyoxyphoshoryl, diaryloxyphosphoryl, phosphoryl, phosphinyl, phenyl, phenyl(C₁-C₃)alkyl-, phenoxy, monocyclic heteroaryl, heteroaryl(C₁-C₃)alkyl-, or heteroaryloxy with 5 or 6 ring atoms, cycloalkyl having 3 to 6 ring carbon atoms, —COOR^{A}, —COR^{A}, —OCOR^{A}, —SO₂R^{A}, —CONR^{A}R^{B}, —CONHNH₂, —SO₂NR^{A}R^{B}, SF₅, —NR^{A}R^{B}, —NHNH₂, —OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl, hydroxy(C₁-C₆)alkyl, or (C₁-C₃)alkoxy(C₁-C₃)alkyl group or, in the case where R^{A} and R^{B} are linked to the same N atom, R^{A} and R^{B} taken together with that nitrogen may form a cyclic amino ring such as morpholinyl, piperidinyl, piperazinyl, or 4-(C₁-C₆)alkyl-piperizinyl.

86-87. (canceled)

88. The compound of claim 65 which is a compound of compound number 124-187 or a salt, racemate, isomer, diastereoisomer, enantiomer, hydrate, solvate, N-oxide, pharmaceutically acceptable derivative or prodrug thereof.

89. A composition comprising a compound according to claim 48 or a salt, racemate, isomer, diastereoisomer, enantiomer, hydrate, solvate, N-oxide, pharmaceutically acceptable derivative or prodrug thereof.

90. A method for the treatment of a bacterial infection comprising administration of a compound according to claim 48 or a pharmaceutically acceptable salt, racemate, isomer, diastereoisomer, enantiomer, hydrate, solvate, N-oxide, pharmaceutically acceptable derivative or prodrug thereof to a patient with said infection.

91-95. (canceled)

96. A composition comprising a compound according to claim 65 or a salt, racemate, isomer, diastereoisomer, enantiomer, hydrate, solvate, N-oxide, pharmaceutically acceptable derivative or prodrug thereof.

97. A method for the treatment of a bacterial infection comprising administration of a compound according to claim 65 or a pharmaceutically acceptable salt, racemate, isomer, diastereoisomer, enantiomer, hydrate, solvate, N-oxide, pharmaceutically acceptable derivative or prodrug thereof to a patient with said infection.
